SEQUENCE LISTING (2 sequences)

SEQ ID NO: 1 - DNA, 718 bp, Human
acaagacatgactgtgatgaggagctgctttcgccaatttaacaccaagaagaattgaggctgcttgggaggaaggccaggaggaacacgagactgagagatgaattttcaacagaggctgcaaagcctgtggactttagccagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctc

SEQ ID NO: 2 - protein, 206 aa, Human
MNFQQRLQSLWTLARPFCPPLLATASQMQMVVLPCLGFTLLLWSQVSGAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

(19) United States
(12) Patent Application Publication
(10) Pub. No.: US 2002/0183271 A1
(43) Pub. Date: Dec. 5, 2002

(54) Methods of treatment involving human MDA-7

(76) Inventors: Sunil Chada, Missouri City, TX (US); Elizabeth Grimm, Houston, TX (US); Abner Mhashilkar, Houston, TX (US); Rajagopal Ramesh, Sugarland, TX (US)

Correspondence Address: Gina N. Shishima, Fulbright & Jaworski L.L.P., Suite 2400, 600 Congress Avenue, Austin, TX, 78701, US

(21) Appl. No.: 10/017,472

(22) Filed: Dec. 7, 2001

Related U.S. Application Data
(60) Provisional application No. 60/254,226, filed on Dec. 7, 2000.

(51) Int. Cl. A61K 48/00; A61K 38/43

(52) U.S. Cl. 514/44; 424/94.1

(57) ABSTRACT

The present invention relates to gene therapy methods for the treatment of human disease. More specifically, the invention is directed to methods for treating a subject with an angiogenesis-related disease. In one embodiment, an adenoviral expression construct comprising a nucleic acid encoding a human MDA-7 protein under the control of a promoter operable in eukaryotic cells, is administered to said patient with an angiogenesis-related disease. The present invention thus provides for treatment of angiogenesis-related disease by through expression of mda-7 and inhibition angiogenesis. Such diseases include cancer.

This application claims the benefit of priority to U.S. Application Serial No. 60/254,226 filed on Dec. 7, 2000, which is hereby incorporated by reference in its entirety.

The government may own rights in the invention pursuant to the specialized Program of Research Excellence (SPORE) in Lung Cancer (P50-CA70907) (J. A. Roth), by Public Health Service grant P01CA78778-01A1, grant number CA73954 from the National Institutes of Health, and grant numbers CA86587 and CA89778 from the National Cancer Institute.

### BACKGROUND OF THE INVENTION

A. Field of the Invention

The present invention relates generally to the field of gene therapy. More particularly, it concerns a method of administering a therapeutic nucleic acid for the treatment of angiogenesis-related disease by inhibiting angiogensis (anti-angiogenic therapy). In one embodiment, the invention relates to the expression of a nucleic acid encoding human mda-7 protein for the treatment of angiogenesis-related disease by inhibiting angiogenesis.

B. Description of Related Art

1. Angiogenesis

Blood vessels are constructed by two processes: vasculogenesis, whereby a primitive vascular network is established during embryogenesis from multipotential mesenchymal progenitors; and angiogenesis, in which preexisting vessels send out capillary sprouts to produce new vessels. Endothelial cells are centrally involved in each process. They migrate, proliferate and then assemble into tubes with tight cell-cell connections to contain the blood (Hanahan, 1997). Angiogenesis occurs when enzymes, released by endothelial cells, and leukocytes begin to erode the basement membrane, which surrounds the endothelial cells, allowing the endothelial cells to protrude through the membrane. These endothelial cells then begin to migrate in response to angiogenic stimuli, forming offshoots of the blood vessels, and continue to proliferate until the off-shoots merge with each other to form the new vessels.

Normally, angiogenesis occurs in humans and animals in a very limited set of circumstances, such as embryonic development, wound healing, and formation of the corpus luteum, endometrium and placenta. However, aberrant angiogenesis is associated with a number of disorders, including, tumor metastasis. In fact, it is commonly believed that tumor growth is dependent upon angiogenic processes. Thus, the ability to increase or decrease angiogenesis has significant implications for clinical situations, such as wound healing (e.g., graft survival) or cancer therapy, respectively.

Several lines of direct evidence now suggest that angiogenesis is essential for the growth and persistence of solid tumors and their metastases (Folkman, 1989; Hon et al., 1991; Kim et al., 1993; Millauer et al., 1994). To stimulate angiogenesis, tumors up-regulate their production of a variety of angiogenic factors, including the fibroblast growth factors (FGF and DTCF) (Kandel et al., 1991) and vascular endothelial cell growth factor/vascular permeability factor (VEGF/VPP). However, many malignant tumors also generate inhibitors of angiogenesis, including angiostatin and thrombospondin (Chen et al., 1995; Good et al., 1990: O'Reilly et al., 1994). It is postulated that the angiogenic phenotype is the result of a net balance between these positive and negative regulators of neovascularization (Good et al., 1990; O'Reilly et al., 1994; Parangi et al., 1996; Rastineiad et al, 1989). Several other endogenous inhibitors of angiogenesis have been identified, although not all are associated with the presence of a tumor. These include, platelet factor 4 (Gupta et al., 1995; Maione et al., 1990), interferon-alpha, interferon-inducible protein 10 (Angiolillo et al., 1995; Strieter et al., 1995), which is induced by interleukin-12 and/or interferon-gamma (Voest et al., 1995), gro-beta (Cao et al., 1995), and the 16 kDa N-terminal fragment of prolactin (Clapp et al., 1993).

2. Angiogenesis-Related Disease

The methods of the present invention are useful for treating endothelial cell-related diseases and disorders. A particularly important endothelial cell process is angiogenesis, the formation of blood vessels, as described above. Angiogenesis-related diseases may be treated using the methods described in present invention to inhibit endothelial cell proliferation. Angiogenesis-related diseases include, but are not limited to, angiogenesis-dependent cancer, including, for example, solid tumors, blood born tumors such as leukemias, and tumor metastases; benign tumors, for example hemangiomas, acoustic neuromas, neurofibromas, trachomas, and pyogenic granulomas; rheumatoid arthritis; psoriasis; ocular angiogenic diseases, for example, diabetic retinopathy, retinopathy of prematurity, macular degeneration, corneal graft rejection, neovascular glaucoma, retrolental fibroplasia, Rubeosis; Osler-Webber Syndrome; myocardial angiogenesis; plaque neovascularization; telangiectasia; hemophiliac joints; angiofibroma; and wound granulation. The endothelial cell proliferation inhibiting methods of the present invention are useful in the treatment of disease of excessive or abnormal stimulation of endothelial cells. These diseases include, but are not limited to, intestinal adhesions, atherosclerosis, scleroderma, and hypertrophic scars, i.e., keloids. They are also useful in the treatment of diseases that have angiogenesis as a pathologic consequence such as cat scratch disease (Rochele minalia quintosa) and ulcers (Helobacter pylori).

3. Cancer

Normal tissue homeostasis is a highly regulated process of cell proliferation and cell death. An imbalance of either cell proliferation or cell death can develop into a cancerous state (Solyanik et al., 1995; Stokke et al., 1997; Mumby and Walter, 1991; Natoli et al., 1998; Magi-Galluzzi et al., 1998). For example, cervical, kidney, lung, pancreatic, colorectal and brain cancer are just a few examples of the many cancers that can result (Erlandsson, 1998; Kolmel, 1998; Mangray and King, 1998; Gertig and Hunter, 1997; Mougin et al., 1998). In fact, the occurrence of cancer is so high that over 500,000 deaths per year are attributed to cancer in the United States alone.

The maintenance of cell proliferation and cell death is at least partially regulated by proto-oncogenes. A proto-oncogene can encode proteins that induce cellular proliferation (e.g., sis, erbB, src, ras and myc), proteins that inhibit cellular proliferation (e.g., Rb, p16, p19, p21, p53, NF1 and WT1) or proteins that regulate programmed cell death (e.g., bcl-2) (Ochi et al., 1998; Johnson and Hamdy, 1998; Liebermann et al., 1998). However, genetic rearrangements or mutations to these proto-oncogenes, results in the conversion of a proto-oncogene into a potent cancer causing oncogene. Often, a single point mutation is enough to transform a proto-oncogene into an oncogene. For example, a point mutation in the p53 tumor suppressor protein results in the complete loss of wild-type p53 function (Vogelstein and Kinzler, 1992; Fulchi et al., 1998) and acquisition of “dominant” tumor promoting function.

Currently, there are few effective options for the treatment of many common cancer types. The course of treatment for a given individual depends on the diagnosis, the stage to which the disease has developed and factors such as age, sex and general health of the patient. The most conventional options of cancer treatment are surgery, radiation therapy and chemotherapy. Surgery plays a central role in the diagnosis and treatment of cancer. Typically, a surgical approach is required for biopsy and to remove cancerous growth. However, if the cancer has metastasized and is widespread, surgery is unlikely to result in a cure and an alternate approach must be taken. Radiation therapy, chemotherapy and immunotherapy are alternatives to surgical treatment of cancer (Mayer, 1998; Ohara, 1998; Ho et al., 1998). Radiation therapy involves a precise aiming of high energy radiation to destroy cancer cells and much like surgery, is mainly effective in the treatment of non-metastasized, localized cancer cells. Side effects of radiation therapy include skin irritation, difficulty swallowing, dry mouth, nausea, diarrhea, hair loss and loss of energy (Curran, 1998; Brizel, 1998).

Chemotherapy, the treatment of cancer with anti-cancer drugs, is another mode of cancer therapy. The effectiveness of a given anti-cancer drug therapy often is limited by the difficulty of achieving drug delivery throughout solid tumors (el-Kareh and Secomb, 1997). Chemotherapeutic strategies are based on tumor tissue growth, wherein the anti-cancer drug is targeted to the rapidly dividing cancer cells. Most chemotherapy approaches include the combination of more than one anti-cancer drug, which has proven to increase the response rate of a wide variety of cancers (U.S. Pat. Nos. 5,824,348; 5,633,016 and 5,798,339, incorporated herein by reference). A major side effect of chemotherapy drugs is that they also affect normal tissue cells, with the cells most likely to be affected being those that divide rapidly (e.g., bone marrow, gastrointestinal tract, reproductive system and hair follicles). Other toxic side effects of chemotherapy drugs are sores in the mouth, difficulty swallowing, dry mouth, nausea, diarrhea, vomiting, fatigue, bleeding, hair loss and infection.

Immunotherapy, a rapidly evolving area in cancer research, is yet another option for the treatment of certain types of cancers. For example, the immune system identifies tumor cells as being foreign and thus are targeted for destruction by the immune system. Unfortunately, the response typically is not sufficient to prevent most tumor growths. However, recently there has been a focus in the area of immunotherapy to develop methods that augment or supplement the natural defense mechanism of the immune system. Examples of immunotherapies currently under investigation or in use are immune adjuvants (e.g., Mycobacterium bovis, Plasmodium falciparum, dinitrochlorobenzene and aromatic compounds) (U.S. Pat. Nos. 5,801,005; 5,739,169; Hui and Hashimoto, 1998; Christodoulides et al., 1998), cytokine therapy (e.g., interferons), and (IL-1, GM-CSF and TNF) (Bukowski et al., 1998; Davidson et al., 1998; Hellstrand et al., 1998) gene therapy (e.g., TNF, IL-1, IL-2, p53) (Qin et al., 1998; Austin-Edward and Villaseca, 1998; U.S. Pat. Nos. 5,830,880 and 5,846,945) and monoclonal antibodies (e.g., anti-ganglioside GM2, anti-HER-2, anti-p185) (Pietras et al., 1998; Hanibuchi et al., 1998; U.S. Pat. No. 5,824,311).

4. Gene Therapy

Gene therapy is an emerging field in biomedical research with a focus on the treatment of disease by the introduction of therapeutic recombinant nucleic acids into somatic cells of patients. Various clinical trials using gene therapies have been initiated and include the treatment of various cancers, AIDS, cystic fibrosis, adenosine deaminase deficiency, cardiovascular disease, Gaucher's disease, rheumatoid arthritis, and others. Currently, adenovirus is the preferred vehicle for the delivery of gene therapy agents. Advantages in using adenovirus as a gene therapy agent are high transduction efficiency, infection of non-dividing cells, easy manipulation of its genome, and low probability of non-homologous recombination with the host genome.

5. Cytokines

IL-10 is a pleiotropic homodimeric cytokine produced by immune system cells, as well as some tumor cells (Howard et al., 1992; Ekmekcioglu et al., 1999). Its immunosuppressive function includes potent inhibition of proinflammatory cytokine synthesis, including that of IFNγ, TNFα, and IL-6 (De Waal Malefyt et al., 1991). The family of IL-10-like cytokines is encoded in a small 195 kb gene cluster on chromosome 1q32, and consists of a number of cellular proteins (IL-10, IL-19, IL-20, MDA-7) with structural and sequence homology to IL-10 (Moore et al., 1990; Kotenko et al., 2000; Gallagher et al., 2000; Blumberg et al., 2001; Dumoutier et al., 2000; Knapp et al., 2000; Jiang et al., 1995a; Jiang et al., 1996). mda-7 has been characterized as an IL-10 family member.

Chromosomal location, transcriptional regulation, murine and rat homologue expression, and putative protein structure all allude to MDA-7 being a cytokine (Knapp et al., 2000; Schaefer et al., 2000; Soo et al., 1999; Zhang et al., 2000). Similar to GM-CSF, TNFα, and IFNγ transcripts, all of which contain AU-rich elements in their 3′UTR targeting mRNA for rapid degradation, MDA-7 has three AREs in its 3′UTR¹⁷. Mda-7 mRNA has been identified in human PBMC (Ekmekcioglu, et al., 2001), and although no cytokine function of human MDA-7 protein has been previously reported, MDA-7 has been designated as IL-24 based on the gene and protein sequence characteristics (NCBI database accession XM_{—}001405). The murine MDA-7 protein homolog FISP (IL-4-Induced Secreted Protein) was reported as a Th2 specific cytokine (Schaefer et al., 2001). Transcription of FISP is induced by TCR and IL-4 receptor engagement and subsequent PKC and STAT6 activation as demonstrated by knockout studies. Expression of FISP was characterized but no function has been attributed yet to this putative cytokine¹⁷. The rat MDA-7 homolog C49a (Mob-5) is 78% homologous to the mda-7 gene and has been linked to wound healing (Soo et al. 1999; Zhang et al., 2000). Mob-5 was also shown to be a secreted protein and a putative cell surface receptor was identified on ras transformed cells (Zhang et al., 2000). Therefore, homologues of the mda-7 gene and the secreted MDA-7 protein are expressed and secreted in various species. However, no data has emerged to show MDA-7 has cytokine activity. Such activity has ramifications for the treatment of a wide variety of diseases and infections by promoting therapeutic immune responses or enhancing immunogenicity of an antigen.

### SUMMARY OF THE INVENTION

It is, therefore, an objective of the present invention to provide a method for treating a patient exhibiting an angiogenesis-related disease comprising administering a therapeutic nucleic acid encoding human MDA-7 protein under the control of a promoter operable in eukaryotic cells, wherein expression of mda-7 inhibits angiogenesis.

In certain embodiments the angiogenesis-related diseases are angiogenesis-dependent cancer, benign tumors, rheumatoid arthritis, psoriasis, ocular angiogenic diseases, Osler-Webber Syndrome, myocardial angiogenesis, plaque neovascularization, telangiectasia, hemophiliac joints, angiofibroma, wound granulation, cat scratch disease, ulcers, intestinal adhesions, atherosclerosis, scleroderma, and/or hypertrophic scars (keloids).

In further embodiments angiogenesis-dependent cancers are further defined as solid tumors, blood born tumors such as leukemias, and/or tumor metastases. In additional embodiments benign tumors are further defined as hemangiomas, acoustic neuromas, neurofibromas, trachomas, and/or pyogenic granulomas. In still further embodiments ocular angiogenic diseases are further defined as diabetic retinopathy, retinopathy of prematurity, macular degeneration, corneal graft rejection, neovascular glaucoma, retrolental fibroplasia, and/or Rubeosis.

The method for treating a patient the present invention comprises the transfer of a nucleic acid encoding all or part of the human MDA-7 protein. Following the administering of the nucleic acid to a patient in need of anti-angiogeneic therapy under control of a promoter active in eukaryotic cells, MDA-7 protein is expressed or taken up by endothelial cells thereby stimulating growth arrest.

In certain preferred embodiments, the angiogenesis-related disease is further defined as cancer. In more preferred embodiments, the cancer is melanoma, non-small cell lung, small-cell lung, lung, hepatocarcinoma, retinoblastoma, astrocytoma, glioblastoma, leukemia, neuroblastoma, head, neck, breast, pancreatic, prostate, renal, bone, testicular, ovarian, mesothelioma, cervical, gastrointestinal, lymphoma, brain, colon or bladder. In still more preferred embodiments said angiogenesis-related diseases is rheumatoid arthritis, inflammatory bowel disease, osteoarthritis, leiomyomas, ademonas, lipomas, hemangiomas, fibromas, vascular occlusion, restenosis, atherosclerosis, pre-neoplastic lesions, carcinoma in situ, oral hairy leukoplakia or psoriasis.

In certain embodiments, the nucleic acid is a viral vector, wherein the viral vector dose is or is at least 10³, 10⁴, 10⁵, 10⁶, 10⁷, 10⁸, 10⁹, 10¹⁰, 10¹¹, 10¹², 10¹³ or higher pfu or viral particles. In more preferred embodiments, the viral vector is an adenoviral vector, a retroviral vector, a vaccinia viral vector, an adeno-associated viral vector, a polyoma viral vector or a herpesviral vector. Most preferably, the viral vector is an adenoviral vector.

In certain embodiments, the promoter is the CMV IE, dectin-1, dectin-2, human CD11c, F4/80, SM22 or MHC class II promoter, however any other promoter that is useful to drive expression of the mda-7 gene of the present invention, such as those set forth herein, is believed to be applicable to the practice of the present invention.

Preferably, the nucleic acid of the present invention is administered by injection. Other embodiments include the administering of the nucleic acid by multiple injections. In certain embodiments, the injection is performed local, regional or distal to a disease or tumor site. In preferred embodiments, the administering of nucleic acid is via continuous infusion, intratumoral injection, intraperitoneal, or intravenous injection. In certain preferred embodiments, the nucleic acid is administered to the tumor bed prior to or after; or both prior to and after resection of the tumor. In preferred embodiments, the nucleic acid is administered to the patient before, during, or after chemotherapy, biotherapy, immunotherapy, surgery or radiotherapy. Preferably the patient is a human. In other embodiments the patient is a cancer patient.

In preferred embodiments, the nucleic acid encodes amino acids from 49 to 206, 75 to 206, 100 to 206, 125 to 206, 150 to 206, 175 to 206, or 182 to 206 of SEQ ID NO:2. In still further embodiments the nucleic acid encodes or encodes at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, or 206 contiguous amino acids of SEQ ID NO:2.

In certain preferred embodiments, the nucleic acid further comprises nucleotides encoding a secretory signal sequence. In more preferred embodiments, the nucleic acid further comprises secretory signal sequence defined as a positively charged N-terminal region in combination with a hydrophobic core.

While in further embodiments, chemotherapy involving at least one DNA damaging agent is implemented in combination with administration of an MDA-7 encoding nucleic acid molecule. The DNA damaging agent may be gamma-irradiation, X-rays, proton-beam irradiation, UV-irradiation, microwaves, electronic emissions, adriamycin, 5-fluorouracil (5FU), etoposide (VP-16), camptothecin, actinomycin-D, mitomycin C, cisplatin (CDDP), or hydrogen peroxide. In further embodiments, the DNA damaging agent is adriamycin. While in other embodiments, the chemotherapy comprises a cisplatin (CDDP), carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxotere, taxol, transplatinum, 5-fluorouracil, vincristin, vinblastin, or methotrexate or any analog or derivative variant thereof. In one aspect of the invention, the chemotherapy comprises tamoxifen, while in another aspect is it comprises adriamycin. Further embodiments involve immunotherapy, such as Herceptin.

In cases involving a cancerous tumor, a combination treatment may involve administration of a nucleic acid molecule encoding MDA-7 polypeptide and tumor resection, which may occur before, after, or during the mda-7 gene therapy administration. If mda-7 treatment occurs after tumor resection, the expression construct or vector encoding MDA-7 may be administered to the tumor bed.

In other embodiments, a method of inhibiting endothelial cell differentiation comprising administering to an endothelial cell a nucleic acid molecule encoding human MDA-7 protein under the control of a promoter operable in eukaryotic cells is described. Alternatively, the mda-7 expression vector can be administered to tumor cells or at a site near or local to a tumor, thereby causing the release of MDA-7 protein. The MDA-7 protein will bind to endothelial cells and inhibit angiongenesis.

Additional embodiments encompass the administration of a chemotherapeutic agent prior, after or before the nucleic acid molecule. In still further embodiments the chemotherapeutic agent is a DNA damaging agent. DNA damaging agent is further defined as gamma-irradiation, X-rays, proton-beam irradiation, UV-irradiation, microwaves, electronic emissions, adriamycin, 5-fluorouracil (5FU), etoposide (VP-16), camptothecin, actinomycin-D, mitomycin C, cisplatin (CDDP), or hydrogen peroxide. While in other embodiments DNA damaging agents are further defined as cisplatin (CDDP), carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxol, transplatinum, 5-fluorouracil, vincristin, vinblastin and methotrexate or any analog or derivative variant thereof.

In certain embodiments the nucleic acid is comprised within a viral vector or in a lipid composition.

Additional embodiments of the invention encompass the use of a purified protein composition comprising MDA-7 protein, truncated versions of MDA-7, and peptides derived from MDA-7 amino acid sequence administered to cells or subjects for the inhibition of angiogenesis.

Other embodiments of the invention concern MDA-7's cytokine activity. The present invention includes methods for promoting an immune response in a subject comprising providing to the subject an effective amount of MDA-7 to promote an immune response. The promotion of an immune response is evidenced by an increase of cytokine expression or activity, proliferation of T cells or a population of T cells (for example, helper, cytotoxic, NK cells) , proliferation of B cells or a population of B cells, cytotoxic T cell activity, or antibody production.

In some embodiments of the invention, an antigen also is provided to the subject, resulting in an immune response against the antigen. The antigen may be a tumor antigen, microbial antigen, viral antigen, or fungal antigen, or a combination thereof. In some embodiments the antigen is a tumor antigen, such as PSA, CEA, MART, MAGE1, MAGE 3, gp100, BAGE, GAGE, TRP-1, TRP-2, or PMSA.

Additional embodiments of the invention include methods of enhancing or improving recovery or methods of reducing damage from traumatic treatment, which is a treatment that causes damage to normal cells. Such damage causes neutropenia, anemia, thrombocytopenia, and lymphopenia, for example. In some embodiments, the traumatic treatment is chemotherapy and/or radiotherapy. It is contemplated that MDA-7 can be administered to a patient who will, is undergoing, or has undergone traumatic treatment. MDA-7 can be provided to a subject before, after or during treatment.

The invention also includes methods of inducing the expression of IL-6, interferon γ(IFNγ), tumor necrosis factor α(TNFα ) by administering to a cell or patient an effective amount of MDA 7 polypeptide or a nucleic acid expressing the MDA-7 polypeptide, whereby induction of a secondary antibody, such as IL-6, IFNγ, or TNFα occurs.

It is contemplated that MDA-7, an antigen, or both can be provided to the subject by administering to the subject an expression construct comprising a nucleic acid sequence encoding at least 10, 20, 30, 40, 50, 60, 70, 80, 90, 100, 110, 120, 130, 140, 150, 160, 170, 180, 190, 200, or 206 contiguous amino acids of SEQ ID NO:2, wherein the nucleic acid sequence is under the transcriptional control of a promoter. A number of promoters are discussed in a later section and are contemplated for use with the invention, though the invention is in no way limited to those promoters. In some embodiments, the expression construct is a viral vector. Viral vectors include an adenovirus vector, an adeno-associated virus vector, a herpesvirus vector, a retrovirus vector, a lentivirus vector, a vaccinia virus vector, or a polyoma vector.

A subject may be given MDA-7 or the antigen more than one time, such as two, three, four times or more. MDA-7 and the antigen may be given at the same time or at different times. Furthermore, it is contemplated that these compounds can be provided to a subject intravenously, directly, intraperitoneally, regionally, systemically, or orally.

It is contemplated that embodiments discussed herein with respect to one method of the invention may be implemented with respect to other methods of the invention.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1. Schematic illustration of Ad-vectors. Replication-deficient human type 5 Adenovirus (Ad5) carrying the mda-7 (or luciferase gene) linked to an internal CMVIE promoter and followed by SV40 polyadenylation (pA) signal were used. In addition, Ad-CMVp(A) (empty vector) was used as control.

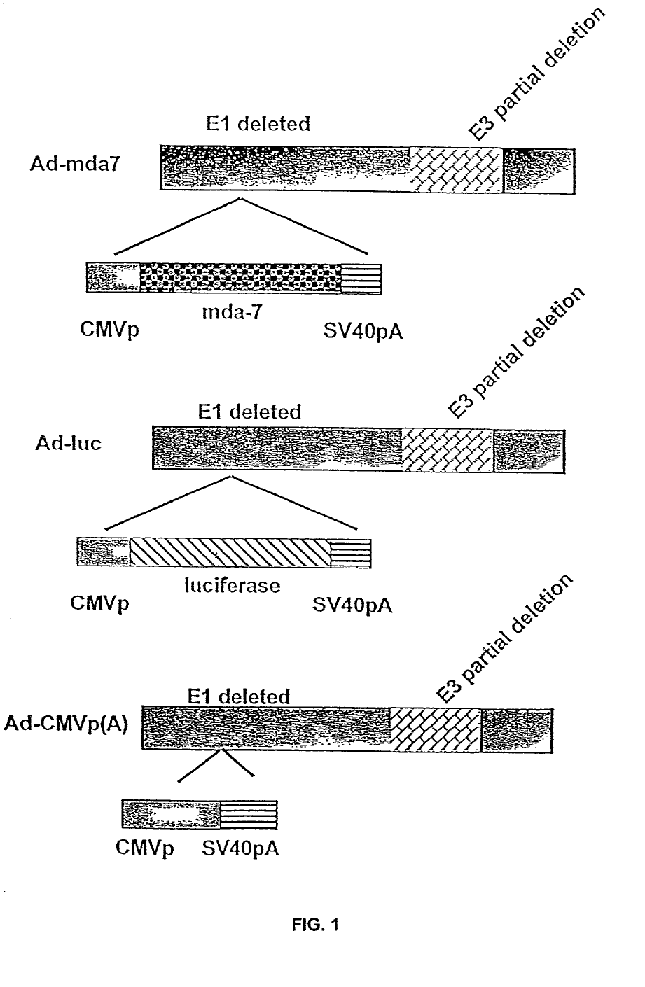

FIG. 2A. T47D cells treated with Ad-mda7 at varying MOIs (viral particle/cell). FIG. 2B. MCF-7 cells treated with Ad-mda7 at varying MOIs (Viral particle/cell).

FIG. 3A. MDA-MB-361 cells treated with Ad-mda7 at varying MOIs (Viral particle/cell). FIG. 3B. BT-20 cells treated with Ad-mda7 at varying MOIs (Viral particle/cell).

FIG. 4A. H1299 cells treated with Ad-mda7 at varying MOIs (Viral particle/cell). FIG. 4B. H322 cells treated with Ad-mda7 at varying MOIs (Viral particle/cell).

FIG. 5A. SW620 cells treated with Ad-mda7 at varying MOIs (Viral particle/cell). FIG. 5B. DLD-1 cells treated with Ad-mda7 at varying MOIs (Viral particle/cell).

FIG. 6A. MJ90 cells treated with Ad-mda7 at varying MOIs (Viral particle/cell). FIG. 6B. HUVEC cells treated with Ad-mda7 at varying MOIs (Viral particle/cell).

FIG. 7. Annexin V assay to determine apoptosis induction after Ad-mda7 transduction in breast cancer cell lines. Three breast cancer cell lines (T47D, MDA-MB-468, MCF-7) were infected with Ad-mda7 or control Ad-CMVp(A) empty vector, and evaluated for apoptosis using Annexin V.

FIG. 8. DLD-1 cells were infected with Ad-mda7 or Ad-luc and 48 hours later examined for Annexin V staining by FACS analysis.

FIG. 9. Panel A shows apoptosis induction in H1299 cells infected with Ad-mda7 or Ad-luc. Cells were evaluated at different time points post-infection using Annexin V staining and FACS analysis. Panel B illustrates apoptosis in DLD-1 cells that were infected with Ad-mda7 or Ad-luc at different time points post-infection (as examined by Annexin V staining and FACS analysis).

FIG. 10. H460 cells were infected with increasing MOIs of Ad-mda7 or Ad-luc and 48 h later processed for MDA7 surface expression and analyzed by FACS.

FIG. 11A. Soluble MDA-7 (sMDA7) kills tumor cells. H1299 cells were challenged with the following samples and percentage dead cells evaluated after 48 hours: 1) Ad-mda7 virus, positive control infected at 1000 Vp/cell; 2) Soluble MDA7 supernatant from H1299 infected cells with Ad-mda-7 (1000 vp/cell); 3) Ad-luc virus, control infected at 1000 Vp/ cell; 4) supernatant from H1299 infected cells with Ad-luc (1000 vp/cell); 5) Ad-p53 virus, positive control infected at 20 Vp/cell; 6) a separate stock of soluble MDA-7 supernatant obtained from 293 cells infected with Ad-mda-7 (sup M, 500 Vp/cell); and 7) a separate stock of soluble MDA-7 supernatant obtained from modified serum-free 293 cells infected with Ad-mda-7 (sup P, 500 Vp/cell). All the supernatants used in this experiment were filtered through a 0.1 micron filter prior to challenge with H1299 cells. FIG. 11B. H1299 cells were challenged with soluble MDA-7 supernatant from four different stocks and percentage dead cells evaluated after 48 hours: 1) 293*NF: Non-filtered supernatant obtained from modified 293 cells (cells were grown in serum-free conditions); 2) 293*F: 0.1 micron filtered supernatant obtained from modified 293 cells; 3) 293F: 0.1 micron filtered supernatant obtained from regular 293 cells (FBS+); and 4) H1299F: 0.1 micron filtered supernatant obtained from H1299 cells. D0 is non-diluted material whereas D1:1; D1:5, D1:10 indicate the dilutions used. Control undiluted supernatant from Ad-luc treated H1299 cells demonstrated 20% dead cells.

FIG. 12. Combination with Tamoxifen. Ad-mda7 has been combined with tamoxifen and evaluated for anti-tumor effects in breast cancer cell lines. The graphs demonstrate that combining these two agents provides superior anti-tumor activity compared to either agent alone.

FIG. 13. Combination with Adriamycin. Ad-mda7 has been combined with adriamycin and evaluated for anti-tumor effects in breast cancer cell lines. The graphs demonstrate that combining these two agents provides superior anti-tumor activity compared to either agent alone.

FIG. 14. Left Panel: MDA-7 protein expression in NSCLC cells and normal lung cells after transduction with Ad-mda7. NHFB-normal human bronchial cells. Right Panel-upper: Effect of Ad-mda7 on growth of NSCLC cells and normal lung cells. Ad-mda7 (circles), PBS (diamonds), Ad-luc (squares). Lower Panel: Cell cycle analysis of NSCLC cells and normal lung cells after transduction with Ad-mda7. Note significant decrease in G1 and increase in G2/M.

FIG. 15. Combination of Ad-mda7 and Herceptin on breast cancer cell lines. Cell lines treated with Ad-mda7 are enhanced in a Her2-expressing cell line as compared to a non-expressing cell line, demonstrating the increased effectiveness of Herceptin on killing cells following contact with Ad-mda7.

FIG. 16. MDA-7 overexpression induces apoptotic tumor cell death and inhibits cell proliferation in vitro. Tumor cells (H1299, A549), and normal human bronchial epithelial cells (NHBE) were infected with Ad-luc or Ad-mda7 (5000 vp/cell). Seventy-two hours after infection, cells were stained with Hoechst 33342 and observed under fluorescence microscopy. Analysis of cell viability by MTT assay at 72 hours after infection showed inhibition of tumor cell proliferation (27% for H1299, and 40% for A549) but not in NHBE.

FIG. 17. Therapeutic effect of Ad-mda7 treatment on subcutaneous human lung cancer xenografts. Subcutaneous H1299 (a) and A549 (b) tumor-bearing mice were divided into three groups (8 animals/group) and treated on alternate days for a total of three doses (5×10⁹ vp/dose), as follows: no treatment (□), Ad-luc (▪) or Ad-mda7 (●). Tumors were measured using calipers, and the statistical significance of tumor volume changes were calculated using the student's t-test. Each time point represents the mean tumor volume for each group. Bars represent standard error.

FIG. 18. mda-7 gene expression and apoptotic cell death following Ad-mda7 treatment in vivo. Subcutaneous H1299 tumors from animals receiving Ad-luc or Ad-mda7 were harvested 48 hours after treatment. Quantitative analysis of tumor tissues demonstrated 15% of tumor cells treated with Ad-mda7 expressing MDA-7 (a) and 17% of tumor cells undergoing apoptosis (b).

FIG. 19. Downregulation of CD31 and upregulation of TRAIL expression by mda-7. Lower CD31 expression was observed in tumors treated with Ad-mda7 (9%) than in tumors receiving no treatment (40%) or Ad-luc (28%) (a). Expression of TRAIL was higher in tumors treated with Ad-mda7 (20%) than in tumors receiving no treatment (1%) or Ad-luc (4%) (b).

FIG. 20. Summary of immunihistochemistry analyses of patients treated intratumorally with Ad-mda7. “Pt” indicates patient. Time indicates the number of hours after injection that tumors were resected for immunohistochemistry. References to MDA-7 indicate positive expression for MDA-7 protein at center of tumor (at injection site) or at the periphery (>1 cm from injection site). TUNEL data is presented as well. Ad-mda7 injection into tumors in humans results in high levels of mda-7 transgene expression and high levels levels of apoptosis induction.

FIG. 21. Summary of DNA PCR data demonstrating high levels of Ad-mda7 DNA in the center of injected lesions in patients. Tumors were injected with Ad-mda7 and at the time indictated, were resected. Approximately 2 mm sections were obtained from the center of each tumor (corresponding to the injection site) and were subjected to quantitative DNA-PCR analysis. Data are plotted as numbers of Ad-mda7 DNA copies per μg total tumor DNA and compared to time of resection (for example, “1.00E+03” indicates 1.00×10³). The background of the assay was approximately 100 copies/μg DNA.

FIG. 22. MDA-7 protein inhibits endothelial differentiation in a dose-dependent manner.

FIG. 23A-B. MDA-7 is a secreted protein. A. Schematic of MDA-7 protein features. C. Hydrophobicity plot of the MDA-7 protein with leader sequence.

FIG. 24. Effect of MDA-7 and IL-10 on inflammatory cytokine secretion from PBMC. Two ml/well (2×10⁶ cells/ml) PBMC were plated in a 24 well plate and cultured untreated or with indicated amounts of MDA-7 (B 2 ng/ml; D,F 20 ng/ml), 5 μg/ml LPS, 5 μg/ml PHA, 500 U/ml IL-10 (˜17 ng/ml; R & D Systems, Minneapolis, Minn.) or combination of IL-10 and MDA-7. Supernatants were harvested at 48 hours and analyzed for cytokine content by ELISA (Endogen, Corp) according to manufacturer's instructions. Data from one representative donor are reported. * Indicates values above standard curve.

FIG. 25. MDA-7 induces IL-1β, IL-12 and GM-CSF secretion from PBMC, which is inhibited by IL-10. Human PBMC were treated with 5 μg/ml LPS, 500 U/ml IL-10, 5 ng/ml MDA-7 or combinations for 48 hours. An equal volume of an S column fraction not containing MDA-7 as determined by Western (an MDA-7 Neg Fraction) was used as a control for buffer/salt content. Supernatants were harvested at 48 hours and analyzed for cytokine content by ELISA (Endogen, Corp) according to manufacturer's instructions. Data from one representative donor are reported. * Indicates value greater than 500 pg/ml IL-1β. ** Actual values are 539 pg/ml IL-12 and 893 pg/ml GM-CSF. Data from one representative donor are reported.

### DESCRIPTION OF ILLUSTRATIVE EMBODIMENTS

As mentioned above, tumor suppressors play an important role in cancer biology. One of these, p53 tumor suppressor proto-oncogene is essential for the maintenance of the non-tumorogenic phenotype of cells (reviewed by Soddu and Sacchi, 1998). Approximately 50% of all cancers have been found to be associated with mutations of the p53 gene, which result in the loss of p53 tumor suppressor properties (Levine et al., 1991; Vogelstein and Kinzler, 1992; Hartmann et al., 1996a; Hartmann et al., 1996b). Mutations in the p53 gene also result in the prolongation of the p53 half-life in cells and the overexpression of p53 protein. In normal cells, p53 is undetectable due to its high turnover rate. The high incidence of cancer related to mutations of the p53 gene has prompted many research groups to investigate p53 as a route of cancer treatment via gene replacement.

MDA-7, another putative tumor suppressor, has been shown to suppress the growth of cancer cells that are p53-wild-type, p53-null and p53-mutant. Also, the upregulation of the apoptosis-related Bax gene in p53 null cells indicates that MDA-7 is capable of using p53-independent mechanisms to induce the destruction of cancer cells. These characteristics suggest that MDA-7 has broad therapeutic potential as an inducer of apoptosis.

The present invention contemplates the treatment of a patient in need of anti-angiogenesis therapy, including cancer, by identifying patients with such diseases and expressing human mda-7 polypeptide by means of nucleic acid transfer. The treatment of such an angiogenesis-related disease in one embodiment involves the intratumoral administration of either human mda-7 expression construct to endothelial cells of the disease related vascular bed. The endothelial cells then express human MDA-7, resulting in the inhibition of angiogenesis.

### A. MDA-7

The cDNA encoding the MDA-7 protein (also referred to mda-7 herein) has been described by Jiang et al., 1995 (WO 95/11986, incorporated herein by reference), where the protein encoded by the mda-7 cDNA was recognized as a potential regulator of melanoma progression. Jiang et al. used a subtractive hybridization technique (Jiang et al., 1995, incorporated herein by reference) to identify genes involved in the regulation of growth and differentiation in human melanoma cells. A cDNA library prepared by subtraction hybridization of cDNAs prepared form actively proliferating human HO-1 melanoma cells against cDNAs prepared from interferon-beta (IFN-β) and mezerin-differentiated human HO-1 melanoma cells was used to identify several melanoma differentiation associated (mda) cDNAs, including mda-7. The expression of mda-7 mRNA is inversely correlated with melanoma progression as demonstrated by increased mRNA levels in normal melanocytes as compared to primary and metastatic melanomas as well as decreased mda-7 mRNA expression in early vertical growth phase melanoma cells selected for enhanced tumor formation in nude mice.

The mda-7 cDNA (SEQ ID NO:1) encodes a novel, evolutionarily conserved protein of 206 amino acids (SEQ ID NO:2) with a predicted size of 23.8 kDa. The deduced amino acid sequence contains a hydrophobic stretch from about amino acid 26 to 45, which has characteristics of a signal sequence. The protein sequence shows no significant homology to known proteins with the exception of a 42 amino acid stretch that is 54% identical to interleukin 10 (IL-10). Structural analysis has determined that MDA-7 (IL-BKW or IL-20) displays the structural characteristics of the cytokine family (WO 98/28425, incorporated herein by reference). The structural characteristics and limited identity across a small stretch of amino acids implies an extracellular function for MDA-7. The expression of MDA-7 is inversely correlated with melanoma progression as demonstrated by increased mRNA levels in normal melanocytes as compared to primary and metastatic melanomas as well as decreased MDA-7 expression in early vertical growth phase melanoma cells selected for enhanced tumor formation in nude mice.

Additional studies have shown that elevated expression of MDA-7 suppressed cancer cell growth in vitro and selectively induced apoptosis in human breast cancer cells as well as inhibiting tumor growth in nude mice (Jiang et al., 1996 and Su et al., 1998). Jiang et al. (1996) report findings that mda-7 is a potent growth suppressing gene in cancer cells of diverse origins including breast, central nervous system, cervix, colon, prostate, and connective tissue. A colony inhibition assay was used to demonstrate that elevated expression of MDA-7 enhanced growth inhibition in human cervical carcinoma (HeLa), human breast carcinoma (MCF-7 and T47D), colon carcinoma (LS174T and SW480), nasopharyngeal carcinoma (HONE-1), prostate carcinoma (DU-145), melanoma (HO-1 and C8161), glioblastome multiforme (GBM-18 and T98G), and osteosarcoma (Saos-2). Mda-7 overexpression in normal cells (HMECs, HBL-100, and CREF-Trans6) showed limited growth inhibition indicating that mda-7 transgene effects are not manifest in normal cells. In summary, growth inhibition by elevated expression of MDA-7 is more effective in vitro in cancer cells than in normal cells.

Su et al. (1998) reported investigations into the mechanism by which MDA-7 suppressed cancer cell growth. The studies reported that ectopic expression of MDA-7 in breast cancer cell lines MCF-7 and T47D induced apoptosis as detected by cell cycle analysis and TUNEL assay without an effect on the normal HBL-100 cells. Western blot analysis of cell lysates from cells infected with adenovirus mda-7 (“Ad-mda-7”) showed an upregulation of the apoptosis stimulating protein BAX. Ad-mda-7 infection elevated levels of BAX protein only in MCF-7 and T47D cells and not normal HBL-100 or HMEC cells. These data lead the investigators to evaluate the effect of ex vivo Ad-mda-7 transduction on xenograft tumor formation of MCF-7 tumor cells. Ex vivo transduction resulted in the inhibition of tumor formation and progression in the tumor xenograft model. The mechanisms by which this novel tumor suppressor molecule acts are beginning to be investigated.

### B. Angiogenesis-Related Disease and Mda-7

The methods of the present invention are useful for treating endothelial cell-related diseases and disorders. A particularly important endothelial cell process is angiogenesis, the formation of blood vessels, as described above. Angiogenesis-related diseases may be treated using the methods described in present invention to inhibit endothelial cell proliferation by elevated expression of MDA-7.

The primary modality for the treatment of cancer using gene therapy is the induction of apoptosis. This can be accomplished by either sensitizing the cancer cells to other agents or inducing apoptosis directly by stimulating intracellular pathways. Other cancer therapies take advantage of the need for the tumor to induce angiogenesis to supply the growning tumor with neccessary nutrients. Endostatin and angiostatin are examples of two such therapies (WO 00/05356 and WO 00/26368).

Applicants have discovered a new method of inhibiting angiogenesis. This new method comprises the administration of a nucleic acid encoding human mda-7. Ad-mda-7 has the ability to inhibit endothelial differentiation when added to proliferating endothelial cells in vitro. The anti-angiogenic effects of elevated mda-7 expression make this molecule an ideal gene therapy treatment for angiogenesis-related diseases, especially cancer. Administration of a nucleic acid encoding mda-7, via viral or non-viral vectors, to anti-angiogenic target cells, which can comprise endothelial cells, as well as administration to tumor cells is contemplated. This combination treatment allows the clinician to not only rely on the direct transduction of a tumor cell but also on the effect of inhibiting angiogenesis. Thus, starving and attacking the tumor by using two separate modalities that may be delivered to different target cell population.

Angiogenesis-related diseases include, but are not limited to, angiogenesis-dependent cancer, including, for example, solid tumors, blood-borne tumors such as leukemias, and tumor metastases; benign tumors, for example hemangiomas. acoustic neuromas, neurofibromas, trachomas, and pyogenic granulomas; rheumatoid arthritis; psoriasis; ocular angiogenic diseases, for example, diabetic retinopathy, retinopathy of prematurity. macular degeneration, corneal graft rejection, neovascular glaucoma, retrolental fibroplasia. Rubeosis; Osler-Webber Syndrome; myocardial angiogenesis; plaque neovascularization; telangiectasia; hemophiliac joints: angiofibroma; and wound granulation. The endothelial cell proliferation inhibiting methods of the present invention are useful in the treatment of disease of excessive or abnormal stimulation of endothelial cells. These diseases include, but are not limited to, intestinal adhesions, atherosclerosis. scleroderma, and hypertrophic scars, i.e., keloids. They are also useful in the treatment of diseases that have angiogenesis as a pathologic consequence such as cat scratch disease (Rochele minalia quintosa) and ulcers (Helobacter pylori).

Cancer has become one of the leading causes of death in the Western world, second only behind heart disease. Current estimates project that one person in three in the U.S. will develop cancer, and that one person in five will die from cancer. Cancers can be viewed as altered cells that have lost the normal growth-regulating mechanisms.

There currently are three major categories of oncogenes, reflecting their different activities. One category of oncogenes encodes proteins that induce cellular proliferation. A second category of oncogenes, called tumor-suppressors genes or anti-oncogenes, function to inhibit excessive cellular proliferation. The third category of oncogenes, either block or induce apoptosis by encoding proteins that regulate programmed cell death.

The cDNA encoding the mda-7 protein has been described by Jiang et al., 1995 (WO 9511986), where the protein encoded by the mda-7 cDNA was recognized as a potential regulator of melanoma progression. Jiang et al. used a subtractive hybridization technique (Jiang and Fisher, 1993) to identify genes involved in the regulation of growth and differentiation in human melanoma cells. A cDNA library prepared by subtraction hybridization of cDNAs prepared form actively proliferating human HO-1 melanoma cells against cDNAs prepared from interferon (IFN-β) and mezerin differentiated human HO-1 melanoma cells was used to identify several melanoma differentiation associated (mda) cDNAs. The cDNA for mda-7 was identified as having elevated expression levels in the differentiated melanoma cells.

The mda-7 cDNA encodes a novel, evolutionarily conserved protein of 206 amino acids with a predicted size of 23.8 kDa. The deduced amino acid sequence contains a hydrophobic stretch from about amino acid 26 to 45. The protein sequence shows no significant homology to known proteins or protein motifs with the exception of a 42 amino acid stretch that is 54% identical to interleukin 10 (IL-10). Structural analysis has determined that mda-7 (IL-BKW or L-20) displays structural characteristics of the cytokine family (WO 9828425). The structural characteristics and limited identity across a small stretch of amino acids implies a potential extracellular function for mda-7.

Additional studies have shown that elevated expression of mda-7 suppressed cancer cell growth and selectively induced apoptosis in human breast cancer cells as well as inhibiting tumor growth in nude mice (Jiang et al., 1996 and Su et al., 1998). Jiang et al. (1996) report findings that mda-7 is a potent growth suppressing gene in cancer cells of diverse origins including breast, central nervous system, cervix, colon, prostate, and connective tissue. A colony inhibition assay was used to demonstrate that elevated expression of MDA-7 enhanced growth inhibition in human cervical carcinoma (HeLa), human breast carcinoma (MCF-7 and T47D), colon carcinoma (LS174T and SW480), nasopharyngeal carcinoma (HONE-1), prostate carcinoma (DU-145), melanoma (HO-1 and C8161), glioblastome multiforme (GBM-18 and T98G), and osteosarcoma (Saos-2). mda-7 overexpressed in normal cells (HMECs, HBL-100, and CREF-Trans6) showed a reduced colony inhibition.

Growth inhibition by elevated expression of mda-7 is more effective in cancer cells than in normal cells. Su et al. (1998) investigated the mechanism by which mda-7 suppressed cancer cell growth. The studies reported that ectopic expression of mda-7 in breast cancer cell lines MCF-7 and T47D induced apoptosis as detected by cell cycle analysis and TUNEL assay without an effect on the normal HBL-100 cells. Western blotting of lysates from cells infected with adenovirus mda-7 showed an upregulation of the apoptosis stimulating protein Bax. Ad-mda-7 infection elevated levels of Bax protein only in MCF-7 and T47D cells and not normal HBL-100 or HMEC cells. These data lead the investigators to evaluate the effect of ex vivo Ad-mda-7 transduction on xenograft tumor formation of MCF-7 tumor cells. Ex vivo transduction resulted in the inhibition of tumor formation and progression in the tumor xenograft model. Mda-7 has been shown to be effective in tumor cell specific apoptotic induction. Thus, one embodiment of the present invention is the treatment of various angiogenesis-related diseases with a mda-7 adenoviral construct.

PCT publication number WO 98/28425 describes a cytokine molecule allegedly related to IL-10. This molecule, designated IL-BKW, appears to be derived from the same gene as mda-7. The mature form of IL-BKW was said to begin at about residue 47 or 49 of the mda-7 coding region, and continue some 158-160 residues, i.e., to residues 206 of the mda-7 sequence. Thus, a preferred molecule would preferably lack all or part of both the putative signal sequence (residues 1-25) and a putative membrane spanning hydrophobic domain (residues 26-45) of full length mda-7.

Truncated molecules of mda-7 are also contemplated. For example, while molecules beginning approximately at mda-7 amino acid residues 46-49 are the largest molecules, further N-terminal truncations are within the scope of the invention. Thus, specifically contemplated are molecules start at residue 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, and 182, and terminate at residue 206. In additional embodiments, residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, and 46 are included with other contiguous residues of MDA-7, as shown in SEQ ID NO:2.

Though not adhering to a particular theory regarding the operability of these constructs, there is a notable amino acid homology of mda-7 to IL-10 and across species in the D-helical region, located at the C-terminus, which is implicated in receptor binding. Thus, molecules preferably containing this 30-35 amino acid region are particularly preferred.

Thus, in one embodiment of the present invention, the treatment of angiogenesis-related disease involves the administration of a therapeutic peptide or polypetpide. In another embodiment, treatment involves administration of a nucleic acid expression construct encoding mda-7 to target, comprising diseased cells or endothelial cells. It is contemplated that the target cells take up the construct, and express the therapeutic polypeptide encoded by nucleic acid, thereby inhibiting differentiation in the target cells. Cells expressing MDA-7 in turn can secrete the protein which may interact with neighboring cells not transduced or infected by an expression construct. In this way the complex interactions needed to extablish new vasculature for the tumor is inhibited and treatment of the tumor accomplished.

In another embodiment of the present invention, it is contemplated that an angiogenesis-related disease may be treated with a MDA-7, or constructs expressing the same. Some of the angiogenesis-related diseases contemplated for treatment in the present invention are psoriasis, rheumatoid arthritis (RA), inflammatory bowel disease (IBD), osteoarthritis (OA) and pre-neoplastic lesions in the lung.

In yet another embodiment, the treatment of a wide variety of cancerous states is within the scope of the invention. For example, melanoma, non-small cell lung, small-cell lung, lung, hepatocarcinoma, retinoblastoma, astrocytoma, glioblastoma, leukemia, neuroblastoma, head, neck, breast, pancreatic, prostate, renal, bone, testicular, ovarian, mesothelioma, cervical, gastrointestinal, lymphoma, brain, colon or bladder. In still more preferred embodiments said angiogenesis-related diseases is rheumatoid arthritis, inflammatory bowel disease, osteoarthritis, leiomyomas, ademonas, lipomas, hemangiomas, fibromas, vascular occlusion, restenosis, atherosclerosis, pre-neoplastic lesions, carcinoma in situ, oral hairy leukoplakia or psoriasis may be the subject of treatment.

### C. Cytokines and Immune Stimulation

Cytokines can promote an immune response to a compound. Because MDA-7 has cytokine activity, this effect can be utilized for therapeutic and preventative methods. It is contemplated that an immune response against any of the antigens described below would effect a therapeutic effect against a disease or condition associated with the antigen or effect a preventative therapy against that disease or condition.

In some embodiments, MDA-7 can be used to promote or enhance an immune response against an antigen associated with a disease or condition. In some embodiments of the invention, antigens may be associated or derived from microbial, fungal, viral, or tumor agents. Examples of microbes from which antigens of the invention may be drawn include, but are not limited to, the 83 or more distinct serotypes of pneumococci, streptococci such as S. pyogenes, S. agalactiae, S. equi, S. canis, S. bovis, S. equinus, S. anginosus, S. sanguis, S. salivarius, S. mitis, S. mutans, other viridans streptococci, peptostreptococci, other related species of streptococci, enterococci such as Enterococcus faecalis, Enterococcus faecium, Staphylococci, such as Staphylococcus epidermidis, Staphylococcus aureus, particularly in the nasopharynx, Hemophilus influenzae, pseudomonas species such as Pseudomonas aeruginosa, Pseudomonas pseudomallei, Pseudomonas mallei, brucellas such as Brucella melitensis, Brucella suis, Brucella abortus, Bordetella pertussis, Neisseria meningitidis, Neisseria gonorrhoeae, Moraxella catarrhalis, Corynebacterium diphtheriae, Corynebacterium ulcerans, Corynebacterium pseudotuberculosis, Corynebacterium pseudodiphtheriticum, Corynebacterium urealyticum, Corynebacterium hemolyticum, Corynebacterium equi, etc. Listeria monocytogenes, Nocordia asteroides, Bacteroides species, Actinomycetes species, Treponema pallidum, Leptospirosa species and related organisms. The invention may also be useful against gram negative bacteria such as Klebsiella pneumoniae, Escherichia coli, Proteus, Serratia species, Acinetobacter, Yersinia pestis, Francisella tularensis, Enterobacter species, Bacteriodes and Legionella species and the like. In addition, the invention may prove useful in controlling protozoan or macroscopic infections by organisms such as Cryptosporidium, Isospora belli, Toxoplasma gondii, Trichomonas vaginalis, Cyclospora species, for example, and for Chlamydia trachomatis and other Chlamydia infections such as Chlamydia psittaci, or Chlamydia pneumoniae, for example.

Examples of viruses against which viral antigens of the invention may be from include, but are not limited to, influenza A, B and C, parainfluenza, paramyxoviruses, Newcastle disease virus, respiratory syncytial virus, measles, mumps, adenoviruses, adenoassociated viruses, parvoviruses, Epstein-Barr virus, rhinoviruses, coxsackieviruses, echoviruses, reoviruses, rhabdoviruses, lymphocytic choriomeningitis, coronavirus, polioviruses, herpes simplex viruses, human immunodeficiency viruses, cytomegaloviruses, papillomaviruses, virus B, varicella-zoster, poxviruses, rubella, rabies, picornaviruses, rotaviruses and Kaposi associated herpes viruses, hepatitis A, B, C, D, E, F, G, and any other hepatitis viruses, West Nile virus, influenza viruses, paopvaviruses, retroviruses, dengue fever viruses, and ebola viruses.

Examples of fungi against which antigens of the invention may be from include, but are not limited to, Pityrosporum orbiculare, Exophiala werneckii, by Piedraia horta, Trichosporon beigelii, Candida albicans, Sporothrix schenckii, Cladophialophora carrionii, Phialophora verrucosa, and two species of Fonsecaea, Pseudallescheria boydii, Madurella mycetomatis, Madurella grisea, Exophiala jeanselmei, and Acremonium falciforme, are Exophiala jeanselmei, Phialophora richardsiae, Bipolaris spicifera, and Wangiella dermatitidis, Histoplasma capsulatum, Coccidioides immitis, P. brasiliensis, Candida, Cryptococcus neoformans, is Aspergillus fumigatus, Pneumocystis carinii, Rhizopus, Rhizomucor, Absidia, and Basidiobolus.

Furthermore, it is contemplated that all or part of MDA-7 may be part of a fusion protein with another cytokine molecule and/or with an antigen against which an immune response is desired. This could be administered to a subject to induce or promote an immune response against the antigen.

MDA-7 can also be administered to a patient in combination with a tumoricidal compound or a compound with a tumor cytostatic effect to enhance the ability of that compound to inhibit or kill tumor cells. Such compounds include tumor suppressors and compounds discussed below under the heading “Combination Therapy.” In some embodiments, the tumoricidal compound is p53, Rb, WT, FHIT, p16, PTEN, APC, DCC, NF-1, NF-2, WT-1, MEN-I, MEN-II, zac1, p73, VHL, MMAC1, DBCCR-1, FCC, rsk-3, p27, or TRAIL.

An immune response against tumor antigens can also be implemented with MDA-7. Tumor antigens include PSA, CEA, MART, MAGE1, MAGE 3, gp100, BAGE, GAGE, TRP-1, TRP-2, or PMSA. Uses for inducing a response against tumor antigens are specifically contemplated and can be found in U.S. Pat. Nos. 5,552,293 and 6,132,980, which are specifically incorporated by reference.

A number of assays are well known to those of skill in the art regarding assaying for induction, promotion, or enhancement of an immune response, some of which are described in an example below and in the references incorporated by reference herein. One assay involves detecting an increase of expression of other cytokines, such as IL-6, TNF, IFN, GM-CSF, CSF, or other IL cytokines. Such cytokines may be administered to a subject in combination with the MDA-7 compositions described herein and any other composition described herein. It is contemplated that any embodiment discussed with respect to MDA-7 and inhibition of angiogenesis or treatment of cancer may be applied to methods of promoting an immune response.

### D. Nucleic Acids, Vectors and Regulatory Signals

The present invention involves nucleic acids, including MDA-7-encoding nucleic acids, nucleic acids identical or complementary to all or part of the sequence of a mda-7 gene, nucleic acids encoding antigens against which an immune response is desired and other therapeutic nucleic acids, as well as nucleic acids constructs and primers.

The present invention concerns polynucleotides or nucleic acid molecules relating to the mda-7 gene and its gene product MDA-7. These polynucleotides or nucleic acid molecules are isolatable and purifiable from mammalian cells. It is contemplated that an isolated and purified MDA-7 nucleic acid molecule, either the secreted or full-length version, that is a nucleic acid molecule related to the mda-7 gene product, may take the form of RNA or DNA. As used herein, the term “RNA transcript” refers to an RNA molecule that is the product of transcription from a DNA nucleic acid molecule. Such a transcript may encode for one or more polypeptides.

As used in this application, the term “polynucleotide” refers to a nucleic acid molecule, RNA or DNA, that has been isolated free of total genomic nucleic acid. Therefore, a “polynucleotide encoding MDA-7” refers to a nucleic acid segment that contains MDA-7 coding sequences, yet is isolated away from, or purified and free of, total genomic DNA and proteins. When the present application refers to the function or activity of a MDA-7-encoding polynucleotide or nucleic acid, it is meant that the polynucleotide encodes a molecule that has the ability to inhibit angiogenesis, suppress tumor growth, kill cancer cells, and/or induce an immune response.

The term “cDNA” is intended to refer to DNA prepared using RNA as a template. The advantage of using a cDNA, as opposed to genomic DNA or an RNA transcript is stability and the ability to manipulate the sequence using recombinant DNA technology (See Sambrook, 1989; Ausubel, 1996). There may be times when the full or partial genomic sequence is preferred. Alternatively, cDNAs may be advantageous because it represents coding regions of a polypeptide and eliminates introns and other regulatory regions.

It also is contemplated that a given MDA-7-encoding nucleic acid or mda-7 gene from a given cell may be represented by natural variants or strains that have slightly different nucleic acid sequences but, nonetheless, encode a MDA-7 polypeptide; a human MDA-7 polypeptide is a preferred embodiment. Consequently, the present invention also encompasses derivatives of MDA-7 with minimal amino acid changes, but that possess the same activity.

The term “gene” is used for simplicity to refer to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, fusion proteins, and mutants. The nucleic acid molecule encoding MDA-7 or another therapeutic polypeptide may comprise a contiguous nucleic acid sequence of the following lengths or at least the following lengths: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 10100, 10200, 10300, 10400, 10500, 10600, 10700, 10800, 10900, 11000, 11100, 11200, 11300, 11400, 11500, 11600, 11700, 11800, 11900, 12000 or more nucleotides, nucleosides, or base pairs. Such sequences may be identical or complementary to SEQ ID NO:1 (MDA-7 encoding sequence).

“Isolated substantially away from other coding sequences” means that the gene of interest forms part of the coding region of the nucleic acid segment, and that the segment does not contain large portions of naturally-occurring coding nucleic acid, such as large chromosomal fragments or other functional genes or cDNA coding regions. Of course, this refers to the nucleic acid segment as originally isolated, and does not exclude genes or coding regions later added to the segment by human manipulation.

In particular embodiments, the invention concerns isolated DNA segments and recombinant vectors incorporating DNA sequences that encode a UGT2B7 protein, polypeptide or peptide that includes within its amino acid sequence a contiguous amino acid sequence in accordance with, or essentially as set forth in, SEQ ID NO:2, corresponding to the MDA-7 designated “human MDA-7.”

The term “a sequence essentially as set forth in SEQ ID NO:2” means that the sequence substantially corresponds to a portion of SEQ ID NO:2 and has relatively few amino acids that are not identical to, or a biologically functional equivalent of, the amino acids of SEQ ID NO:2.

The term “biologically functional equivalent” is well understood in the art and is further defined in detail herein. Accordingly, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99%; of amino acids that are identical or functionally equivalent to the amino acids of SEQ ID NO:2 will be sequences that are “essentially as set forth in SEQ ID NO:2” provided the biological activity of the protein is maintained. In particular embodiments, the biological activity of a MDA-7 protein, polypeptide or peptide, or a biologically functional equivalent, comprises inhibiting angiogenesis, inhibiting or killing cancer cells, inducing apoptosis, and/or inducing an immune response. In certain other embodiments, the invention concerns isolated DNA segments and recombinant vectors that include within their sequence a nucleic acid sequence essentially as set forth in SEQ ID NO:1. The term “essentially as set forth in SEQ ID NO:1” is used in the same sense as described above and means that the nucleic acid sequence substantially corresponds to a portion of SEQ ID NO:1 and has relatively few codons that are not identical, or functionally equivalent, to the codons of SEQ ID NO:1. Again, DNA segments that encode proteins, polypeptide or peptides exhibiting MDA-7 activity will be most preferred.

In particular embodiments, the invention concerns isolated nucleic acid segments and recombinant vectors incorporating DNA sequences that encode MDA-7 polypeptides or peptides that include within its amino acid sequence a contiguous amino acid sequence in accordance with, or essentially corresponding to MDA-7 polypeptides.

Vectors of the present invention are designed, primarily, to transform endothelial cells with a therapeutic mda-7 gene under the control of regulated eukaryotic promoters (i.e., inducible, repressable, tissue specific). Also, the vectors may contain a selectable marker if, for no other reason, to facilitate their manipulation in vitro. However, selectable markers may play an important role in producing recombinant cells.

Tables 1 and 2, below, list a variety of regulatory signals for use according to the present invention.

TABLE 1
Inducible Elements

| Element | Inducer | References |
|---|---|---|
| MT II | Phorbol Ester (TFA) | Palmiter et al., 1982; Haslinger and |
| | Heavy metals | Karin, 1985; Searle et al., 1985; |
| | | Stuart et al., 1985; Imagawa et al., |
| | | 1987; Karin et al., 1987; Angel |
| | | et al., 1987b; McNeall et al., 1989 |
| MMTV (mouse | Glucocorticoids | Huang et al., 1981; Lee et al., 1981; |
| mammary tumor virus) | | Majors and Varmus, 1983; |
| | | Yamamoto et al., 1983; Lee et al., |
| | | 1984; Ponta et al., 1985; Sakai |
| | | et al., 1986 |
| β-Interferon | poly(rI)X | Tavernier et al., 1983 |
| | poly(rc) | |
| Adenovirus 5 E2 | Ela | Imperiale and Nevins, 1984 |
| Collagenase | Phorbol Ester (TPA) | Angel et al., 1987a |
| Stromelysin | Phorbol Ester (TPA) | Angel et al., 1987b |
| SV40 | Phorbol Ester (TFA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | Ela, SV40 Large T Antigen | Taylor et al., 1989; Taylor and |
| | | Kingston, 1990a,b |
| Proliferin | Phorbol Ester-TPA | Mordacq and Linzer, 1989 |
| Tumor Necrosis Factor | PMA | Hensel et al., 1989 |
| Thyroid Stimulating | Thyroid Hormone | Chatterjee et al., 1989 |
| Hormone α Gene | | |

TABLE 2
Other Promoter/Enhancer Elements

| Promoter/Enhancer | References |
|---|---|
| Immunoglobulin Heavy Chain | Banerji et al., 1983; Gillies et al., 1983; Grosschedl and |
| | Baltimore, 1985; Atchinson and Perry, 1986, 1987; Imler |
| | et al., 1987; Neuberger et al., 1988; Kiledjian et al., 1988; |
| Immunoglobulin Light Chain | Queen and Baltimore, 1983; Picard and Schaffner, 1985 |
| T-Cell Receptor | Luria et al., 1987, Winoto and Baltimore, 1989; Redondo |
| | et al., 1990 |
| HLA DQ α and DQ β | Sullivan and Peterlin, 1987 |
| β-Interferon | Goodbourn et al., 1986; Fujita et al., 1987; Goodbourn |
| | and Maniatis, 1985 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRα | Sherman et al., 1989 |
| β-Actin | Kawamoto et al., 1988; Ng et al., 1989 |
| Muscle Creatine Kinase | Jaynes et al., 1988; Horlick and Benfield, 1989; Johnson |
| | et al., 1989a |
| Prealbumin (Transthyretin) | Costa et al., 1988 |
| Elastase I | Omitz et al., 1987 |
| Metallothionein | Karin et al., 1987; Culotta and Hamer, 1989 |
| Collagenase | Pinkert et al., 1987; Angel et al., 1987 |
| Albumin Gene | Pinkert et al., 1987, Tronche et al., 1989, 1990 |
| α-Fetoprotein | Godbout et al., 1988; Campere and Tilghman, 1989 |
| γ-Globin | Bodine and Ley, 1987; Perez-Stable and Constantini, |
| | 1990 |
| β-Globin | Trudel and Constantini, 1987 |
| c-fos | Cohen et al., 1987 |
| c-HA-ras | Triesman, 1985; Deschamps et al., 1985 |
| Insulin | Edlund et al., 1985 |
| Neural Cell Adhesion Molecule | Hirsch et al., 1990 |
| (NCAM) | |
| a_{1-Antitrypain} | Latimer et al., 1990 |
| H2B (TH2B) Histone | Hwang et al., 1990 |
| Mouse or Type I Collagen | Rippe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| Duchenne Muscular Dystrophy | Klamut et al., 1990 |
| SV40 | Banerji et al., 1981; Moreau et al., 1981; Sleigh and |
| | Lockett, 1985; Firak and Subramanian, 1986; Herr and |
| | Clarke, 1986; Imbra and Karin, 1986; Kadesch and Berg, |
| | 1986; Wang and Calame, 1986; Ondek et al., 1987; Kuhl |
| | et al., 1987 Schaffner et al., 1988 |
| Polyoma | Swartzendruber and Lehman, 1975; Vasseur et al., 1980; |
| | Katinka et al., 1980, 1981; Tyndell et al., 1981; Dandolo |
| | et al., 1983; Hen et al., 1986; Sakai et al., 1988; |
| | Campbell and Villarreal, 1988 |
| Retroviruses | Kriegler and Botchan, 1983; Kriegler et al., 1984a,b; |
| | Bosze et al., 1986; Miksicek et al., 1986; Celander and |
| | Haseltine, 1987; Thiesen et al., 1988; Celander et al., |
| | 1988; Chol et al., 1996; Reisman and Rotter, 1989 |
| Papilloma Virus | Campo et al., 1983; Lusky et al., 1983; Spandidos and |
| | Wilkie, 1983; Spalholz et al., 1985; Lusky and Botchan, |
| | 1986; Cripe et al., 1987; Gloss et al., 1987; Hirochika |
| | et al., 1987, Stephens and Hentschel, 1987 |
| Hepatitis B Virus | Bulla and Siddiqui, 1988; Jameel and Siddiqui, 1986; |
| | Shaul and Ben-Levy, 1987; Spandau and Lee, 1988 |
| Human Immunodeficiency Virus | Muesing et al., 1987; Hauber and Cullan, 1988; |
| | Jakobovits et al., 1988; Feng and Holland, 1988; Takebe |
| | et al., 1988; Berkhout et al., 1989; Laspia et al., 1989; |
| | Sharp and Marciniak, 1989; Braddock et al., 1989 |
| Cytomegalovirus | Weber et al., 1984; Boshart et al., 1985; Foecking and |
| | Hofstetter, 1986 |
| Gibbon Ape Leukemia Virus | Holbrook et al., 1987; Quinn et al., 1989 |

The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation.

The term “promoter” will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between elements is flexible, so that promoter function is preserved when elements are inverted or moved relative to one another. In the tk promoter, the spacing between elements can be increased to 50 bp apart before activity begins to decline. Depending on the promoter, it appears that individual elements can function either co-operatively or independently to activate transcription.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Aside from this operational distinction, enhancers and promoters are very similar entities.

Promoters and enhancers have the same general function of activating transcription in the cell. They are often overlapping and contiguous, often seeming to have a very similar modular organization. Taken together, these considerations suggest that enhancers and promoters are homologous entities and that the transcriptional activator proteins bound to these sequences may interact with the cellular transcriptional machinery in fundamentally the same way.

Preferred for use in the present invention is the cytomegalovirus (CMV) promoter. This promoter is commercially available from Invitrogen in the vector pcDNAIII, which is preferred for use in the present invention. Also contemplated as useful in the present invention are the dectin-1 and dectin-2 promoters. Below are a list of additional viral promoters, cellular promoters/enhancers and inducible promoters/enhancers that could be used in combination with the present invention. Additionally any promoter/enhancer combination (as per the Eukaryotic Promoter Data Base EPDB) could also be used to drive expression of structural genes encoding oligosaccharide processing enzymes, protein folding accessory proteins, selectable marker proteins or a heterologous protein of interest.

Another signal that may prove useful is a polyadenylation signal. Such signals may be obtained from the human growth hormone (hGH) gene, the bovine growth hormone (BGH) gene, or SV40.

The use of internal ribosome binding sites (IRES) elements are used to create multigene, or polycistronic, messages. IRES elements are able to bypass the ribosome scanning model of 5-methylatd cap-dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

In any event, it will be understood that promoters are DNA elements which when positioned functionally upstream of a gene leads to the expression of that gene. Most transgene constructs of the present invention are functionally positioned downstream of a promoter element.

### E. Gene Transfer

1. Viral Transformation

a. Adenoviral Infection

One method for delivery of the recombinant DNA involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a recombinant gene construct that has been cloned therein.

The vector comprises a genetically engineered form of adenovirus. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) have disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

The adenovirus vector may be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

b. Retroviral Infection

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, packaging cell lines are available that should greatly decrease the likelihood of recombination (Markowitz et al., 1988; Hersdorffer et al., 1990).

c. AAV Infection

Adeno-associated virus (AAV) is an attractive vector system for use in the present invention as it has a high frequency of integration and it can infect nondividing cells, thus making it useful for delivery of genes into mammalian cells in tissue culture (Muzyczka, 1992). AAV has a broad host range for infectivity (Tratschin et al., 1984; Laughlin et al., 1986; Lebkowski et al., 1988; McLaughlin et al., 1988), which means it is applicable for use with the present invention. Details concerning the generation and use of rAAV vectors are described in U.S. Pat. Nos. 5,139,941 and 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Shelling and Smith, 1994; Yoder et al., 1994; Zhou et al., 1994; Hermonat and Muzyczka, 1984; Tratschin et al., 1985; McLaughlin et al., 1988) and genes involved in human diseases (Flotte et al., 1992; Luo et al., 1994; Ohi et al., 1990; Walsh et al., 1994; Wei et al., 1994). Recently, an AAV vector has been approved for phase I human trials for the treatment of cystic fibrosis.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild-type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild-type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994a; Clark et al., 1995). Cell lines carrying the rAAV DNA as an integrated provirus can also be used (Flotte et al., 1995).

d. Other Viral Vectors

Other viral vectors may be employed as constructs in the present invention. Vectors derived from viruses such as vaccinia virus (Ridgeway, 1988; Baichwal and Sugden, 1986; Coupar et al., 1988) and herpesviruses may be employed. They offer several attractive features for various mammalian cells (Friedmann, 1989; Ridgeway, 1988; Baichwal and Sugden, 1986; Coupar et al., 1988; Horwich et al., 1990).

A molecularly cloned strain of Venezuelan equine encephalitis (VEE) virus has been genetically refined as a replication competent vaccine vector for the expression of heterologous viral proteins (Davis et al., 1996). Studies have demonstrated that VEE infection stimulates potent CTL responses and has been sugested that VEE may be an extremely useful vector for immunizations (Caley et al., 1997). It is contemplated in the present invention, that VEE virus may be useful in targeting dendritic cells.

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. (1991) recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In still further embodiments of the present invention, the nucleic acid encoding extracellular human MDA-7 to be delivered is housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

For example, to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

2. Non-Viral Delivery

In addition to viral delivery of the nucleic acid encoding extracellular mda-7 protein, the following are additional methods of recombinant gene delivery to a given host cell and are thus considered in the present invention.

a. Electroporation

In certain preferred embodiments of the present invention, the gene construct is introduced into the dendritic cells via electroporation. Electroporation involves the exposure of a suspension of cells and DNA to a high-voltage electric discharge.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

It is contemplated that electroporation conditions for endothelial cells from different sources may be optimized. One may particularly wish to optimize such parameters as the voltage, the capacitance, the time and the electroporation media composition. The execution of other routine adjustments will be known to those of skill in the art.

b. Particle Bombardment

Another embodiment of the invention for transferring a naked DNA construct into cells involves particle bombardment. This method depends on the ability to accelerate DNA-coated microprojectiles to a high velocity allowing them to pierce cell membranes and enter cells without killing them (Klein et al., 1987). The microprojectiles used have consisted of biologically inert substances such as tungsten, platinum or gold beads.

It is contemplated that in some instances DNA precipitation onto metal particles would not be necessary for DNA delivery to a recipient cell using particle bombardment. It is contemplated that particles may contain DNA rather than be coated with DNA. Hence it is proposed that DNA-coated particles may increase the level of DNA delivery via particle bombardment but are not, in and of themselves, necessary.

Several devices for accelerating small particles have been developed. One such device relies on a high voltage discharge to generate an electrical current, which in turn provides the motive force (Yang et al., 1990). Another method involves the use of a Biolistic Particle Delivery System, which can be used to propel particles coated with DNA through a screen, such as stainless steel or Nytex screen, onto a filter surface covered with cells in suspension. The screen disperses the particles so that they are not delivered to the recipient cells in large aggregates. It is believed that a screen intervening between the projectile apparatus and the cells to be bombarded reduces the size of projectile aggregates and may contribute to a higher frequency of transformation by reducing the damage inflicted on the recipient cells by projectiles that are too large.

For the bombardment, cells in suspension are preferably concentrated on filters, or alternatively on solid culture medium. The cells to be bombarded are positioned at an appropriate distance below the macroprojectile stopping plate. If desired, one or more screens are also positioned between the acceleration device and the cells to be bombarded.

In bombardment transformation, one may optimize the prebombardment culturing conditions and the bombardment parameters to yield the maximum numbers of stable transformants. Both the physical and biological parameters for bombardment are important in this technology. Physical factors are those that involve manipulating the DNA/microprojectile precipitate or those that affect the flight and velocity or either the macro- or microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmids. It is believed that pre-bombardment manipulations are especially important for successful transformation of primordial germ cells.

Accordingly, it is contemplated that one may wish to adjust various of the bombardment parameters in small scale studies to fully optimize the conditions. One may particularly wish to adjust physical parameters such as gap distance, flight distance, tissue distance and helium pressure. One also may optimize the trauma reduction factors by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation. The execution of other routine adjustments will be known to those of skill in the art.

c. Calcium Phosphate Co-Precipitation or DEAE-Dextran Treatment

In other embodiments of the present invention, the transgenic construct is introduced to the cells using calcium phosphate co-precipitation. Mouse primordial germ cells have been transfected with the SV40 large T antigen, with excellent results (Watanabe et al., 1997). Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

In another embodiment, the expression construct is delivered into the cell using DEAE-dextran followed by polyethylene glycol. In this manner, reporter plasmids were introduced into mouse myeloma and erythroleukemia cells (Gopal, 1985).

d. Direct Microinjection or Sonication Loading

Further embodiments of the present invention include the introduction of the nucleic acid construct by direct microinjection or sonication loading. Direct microinjection has been used to introduce nucleic acid constructs into Xenopus oocytes (Harland and Weintraub, 1985), and LTK- fibroblasts have been transfected with the thymidine kinase gene by sonication loading (Fechheimer et al., 1987).

e. Lipid Mediated Transformation

In a further embodiment of the invention, the gene construct may be entrapped in a liposome or lipid formulation. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a gene construct complexed with Lipofectamine (Gibco BRL).

Lipid-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of lipid-mediated delivery and expression of foreign DNA in cultured chick embryo, HeLa and hepatoma cells.

Lipid based non-viral formulations provide an alternative to adenoviral gene therapies. Although many cell culture studies have documented lipid based non-viral gene transfer, systemic gene delivery via lipid based formulations has been limited. A major limitation of non-viral lipid based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in lipid vehicle stability in the presence and absence of serum proteins. The interaction between lipid vehicles and serum proteins has a dramatic impact on the stability characteristics of lipid vehicles (Yang and Huang, 1997). Cationic lipids attract and bind negatively charged serum proteins. Lipid vehicles associated with serum proteins are either dissolved or taken up by macrophages leading to their removal from circulation. Current in vivo lipid delivery methods use subcutaneous, intradermal, intratumoral, or intracranial injection to avoid the toxicity and stability problems associated with cationic lipids in the circulation. The interaction of lipid vehicles and plasma proteins is responsible for the disparity between the efficiency of in vitro (Felgner et al., 1987) and in vivo gene transfer (Zhu et al., 1993; Philip et al., 1993; Solodin et al., 1995; Liu et al., 1995; Thierry et al., 1995; Tsukamoto et al., 1995; Aksentijevich et al., 1996).

Recent advances in lipid formulations have improved the efficiency of gene transfer in vivo (Smyth-Templeton et al., 1997; WO 98/07408). A novel lipid formulation composed of an equimolar ratio of 1,2-bis(oleoyloxy)-3-(trimethyl ammonio)propane (DOTAP) and cholesterol significantly enhances systemic in vivo gene transfer, approximately 150-fold. The DOTAP:cholesterol lipid formulation is said to form a unique structure termed a “sandwich liposome”. This formulation is reported to “sandwich” DNA between an invaginated bi-layer or ‘vase’ structure. Beneficial characteristics of these lipid structures include a positive colloidal stabilization by cholesterol, two dimensional DNA packing and increased serum stability.

The production of lipid formulations often is accomplished by sonication or serial extrusion of liposomal mixtures after (I) reverse phase evaporation (II) dehydration-rehydration (III) detergent dialysis and (IV) thin film hydration. Once manufactured, lipid structures can be used to encapsulate compounds that are toxic (chemotherapeutics) or labile (nucleic acids) when in circulation. Lipid encapsulation has resulted in a lower toxicity and a longer serum half-life for such compounds (Gabizon et al., 1990). Numerous disease treatments are using lipid based gene transfer strategies to enhance conventional or establish novel therapies, in particular therapies for treating angiogenesis-related diseasess.

In certain embodiments of the invention, the lipid vehicle may be complexed with a hemagglutinating virus (HVJ). This has been shown to facilitate fusion with the cell membrane and promote cell entry of lipid-encapsulated DNA (Kaneda et al., 1989). In other embodiments, the lipid vehicle may be complexed or employed in conjunction with nuclear non-histone chromosomal proteins (HMG-1) (Kato et al., 1991). In yet further embodiments, the lipid vehicle may be complexed or employed in conjunction with both HVJ and HMG-1.

### F. Pharmaceutical Formulations and Delivery

In a preferred embodiment of the present invention, a method of treatment for a angiogenesis-related diseases by the delivery of an expression construct encoding human mda-7 protein is contemplated. Angiogenesis-related diseases that are most likely to be treated in the present invention are those that result from mutations in an oncogene and the reduced expression of a wild-type protein in the endothelial cells. Examples of angiogenesis-related diseases contemplated for treatment include lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, breast cancer, bladder cancer and any other angiogenesis-related diseasess that may be treated by administering a nucleic acid encoding human mda-7 protein.

An effective amount of the pharmaceutical composition, generally, is defined as that amount sufficient to detectably and repeatedly to ameliorate, reduce, minimize or limit the extent of the disease or its symptoms. More rigorous definitions may apply, including elimination, eradication or cure of disease.

Preferably, patients will have adequate bone marrow function (defined as a peripheral absolute granulocyte count of >2,000/mm³ and a platelet count of 100,000/mm³), adequate liver function (bilirubin<1.5 mg/dl) and adequate renal function (creatinine<1.5 mg/dl).

1. Administration

To kill cells, inhibit cell growth, inhibit metastasis, decrease tumor or tissue size and otherwise reverse or reduce the malignant phenotype of tumor cells, using the methods and compositions of the present invention, one would generally contact a endothelial cell with the therapeutic expression construct. The routes of administration will vary, naturally, with the location and nature of the lesion, and include, e.g., intradermal, parenteral, intravenous, intramuscular, intranasal, and oral administration and formulation.

Intratumoral injection, or injection into the tumor vasculature is specifically contemplated for discrete, solid, accessible tumors. Local, regional or systemic administration also may be appropriate. For tumors of >4 cm, the volume to be administered will be about 4-10 ml (preferably 10 ml), while for tumors of <4 cm, a volume of about 1-3 ml will be used (preferably 3 ml). Multiple injections delivered as single dose comprise about 0.1 to about 0.5 ml volumes. The viral particles may advantageously be contacted by administering multiple injections to the tumor, spaced at approximately 1 cm intervals.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising mda-7 or an mda-7-encoding construct. The perfusion may be continued post-resection, for example, by leaving a catheter implanted at the site of the surgery. Periodic post-surgical treatment also is envisioned.

Continuous administration also may be applied where appropriate, for example, where a tumor is excised and the tumor bed is treated to eliminate residual, microscopic disease. Delivery via syringe or catherization is preferred. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

Treatment regimens may vary as well, and often depend on tumor type, tumor location, disease progression, and health and age of the patient. Obviously, certain types of tumor will require more aggressive treatment, while at the same time, certain patients cannot tolerate more taxing protocols. The clinician will be best suited to make such decisions based on the known efficacy and toxicity (if any) of the therapeutic formulations.

In certain embodiments, the tumor being treated may not, at least initially, be resectable. Treatments with therapeutic viral constructs may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

A typical course of treatment, for a primary tumor or a post-excision tumor bed, will involve multiple doses. Typical primary tumor treatment involves a 6 dose application over a two-week period. The two-week regimen may be repeated one, two, three, four, five, six or more times. During a course of treatment, the need to complete the planned dosings may be re-evaluated.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Unit dose of the present invention may conveniently be described in terms of plaque forming units (pfu) or viral particles for a viral construct. Unit doses range from 10³, 10⁴, 10⁵, 10⁶, 10⁷, 10⁸, 10⁹, 10¹⁰, 10¹¹, 10¹², 10¹³ pfu or viral particles (vp) and higher.

Protein may be administered to a patient in doses of or of at least 0.01. 0.05, 0.1, 0.2, 0.3, 0.4, 0.5, 0.6, 0.7, 0.8, 0.9, 1.0, 2.0, 3.0, 4.0, 5.0, 6.0, 7.0, 8.0. 9.0, 10, 15, 20, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, 10000 or more ng/ml.

2. Injectable Compositions and Formulations

The preferred method for the delivery of an expression construct encoding human mda-7 protein to endothelial cells in the present invention is via intratumoral injection. However, the pharmaceutical compositions disclosed herein may alternatively be administered parenterally, intravenously, intradermally, intramuscularly, or even intraperitoneally as described in U.S. Pat. Nos. 5,543,158; 5,641,515 and 5,399,363 (each specifically incorporated herein by reference in its entirety).

Injection of nucleic acid constructs may be delivered by syringe or any other method used for injection of a solution, as long as the expression construct can pass through the particular gauge of needle required for injection. A novel needeless injection system has recently been described (U.S. Pat. No. 5,846,233) having a nozzle defining an ampule chamber for holding the solution and an energy device for pushing the solution out of the nozzle to the site of delivery. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media which can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

### G. Combination Treatments

In order to increase the effectiveness of MDA-7 polypeptide, or expression construct coding therefor, it may be desirable to combine these compositions with other agents effective in the treatment of angiogenesis-related diseases. These compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver et al., 1992). In the context of the present invention, it is contemplated that mda-7 gene therapy could be used similarly in conjunction with chemo- or radiotherapeutic intervention, in addition to other pro-apoptotic or cell cycle regulating agents.

Alternatively, the gene therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, gene therapy is “A” and the secondary agent, such as radio- or chemotherapy, is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of the therapeutic expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described endothelial cell therapy.

1. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves, proton beam irradiation (U.S. Pat. Nos. 5,760,395 and 4,870,287) and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

In 1945, R. R. Wilson proposed the use of proton beams in the treatment of cancer. The advantage of protons in such treatment resides in the following physical characteristics (1) the radiation dose delivered by a proton penetrating tissue rises as the proton slows down, reaching a maximum near its stopping point (“Bragg peak” ), and is zero beyond the stopping point, (2) protons in a monoenergetic beam have nearly the same range and therefore deliver a maximum dose at the same depth, and (3) protons being relatively heavy do not deviate much from a straight line as they come to rest.

To realize the full potential of the proton beam in the treatment of cancer and other diseases responsive to radiation treatment, it is necessary for the physician to known the exact location of the site to be treated and the characteristics of the tissue overlying the treatment site. It is only with advent of new imaging techniques such as computed tomography (CT scanning) and magnetic resonance imaging (MRI) that such information is now available with the required accuracy. Proton therapy for the treatment of cancer patients is now feasible.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

3. Genes

In yet another embodiment, the secondary treatment is a secondary gene therapy. Delivery of a vector encoding MDA-7 in conjuction with a second vector encoding one of the following gene products will have a combined anti-hyperproliferative effect on target tissues. Alternatively, a single vector encoding both genes may be used.

a. Inducers of Cellular Proliferation

The proteins that induce cellular proliferation further fall into various categories dependent on function. The commonality of all of these proteins is their ability to regulate cellular proliferation. For example, a form of PDGF, the sis oncogene is a secreted growth factor. Oncogenes rarely arise from genes encoding growth factors, and at the present, sis is the only known naturally occurring oncogenic growth factor. In one embodiment of the present invention, it is contemplated that anti-sense mRNA directed to a particular inducer of cellular proliferation is used to prevent expression of the inducer of cellular proliferation.

The proteins fms, erbA, erbB and neu are growth factor receptors. Mutations to these receptors result in loss of regulatable function. For example, a point mutation affecting the transmembrane domain of the neu receptor protein results in the neu oncogene. The erbA oncogene is derived from the intracellular receptor for thyroid hormone. The modified oncogenic erbA receptor is believed to compete with the endogenous thyroid hormone receptor, causing uncontrolled growth.

The largest class of oncogenes are the signal transducing proteins (e.g., src, abl and ras) are signal transducers. The protein src, is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins jun, fos and mye are proteins that directly exert their effects on nuclear functions as transcription factors.

b. Inhibitors of Cellular Proliferation

The tumor suppressor oncogenes function to inhibit excessive cellular proliferation. The inactivation of these genes results destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently-mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G1. The activity of this enzyme may be to phosphorylate Rb at late G1. The activity of CDK4 is controlled by an activating subunit, D-type cyclin, and by an inhibitory subunit, the p16INK4 has been biochemically characterized as a protein that specifically binds to and inhibits CDK4, and thus may regulate Rb phosphorylation (Serrano et al., 1993; Serrano et al., 1995). Since the p16INK4 protein is a CDK4 inhibitor (Serrano, 1993), deletion of this gene may increase the activity of CDK4, resulting in hyperphosphorylation of the Rb protein. p16 also is known to regulate the function of CDK6.

p16INK4 belongs to a newly described class of CDK-inhibitory proteins that also includes p16B, p19, p21WAF1, and p27KIP1. The p16INK4 gene maps to 9p21, a chromosome region frequently deleted in many tumor types. Homozygous deletions and mutations of the p16INK4 gene are frequent in human tumor cell lines. This evidence suggests that the p16INK4 gene is a tumor suppressor gene. This interpretation has been challenged, however, by the observation that the frequency of the p16INK4 gene alterations is much lower in primary uncultured tumors than in cultured cell lines (Caldas et al., 1994; Cheng et al, 1994; Hussussian et al., 1994; Kamb et al., 1994; Kamb et al., 1994; Mori et al, 1994; Okamoto et al., 1994; Nobori et al., 1995; Orlow et al., 1994; Arap et al., 1995). Restoration of wild-type p16INK4 function by transfection with a plasmid expression vector reduced colony formation by some human cancer cell lines (Okamoto, 1994; Arap, 1995).

Other genes that may be employed according to the present invention include RB, APC, DCC, NF-1, NF-2, WT-1, MEN-I, MEN-II, zacl, p73, VHL, MMAC1/PTEN, DBCCR-1, FCC, rsk-3, p27, p27/p16 fusions, p21/p27 fusions, anti-thrombotic genes (e.g., COX-1, TFPI), PGS, genes involved in angiogenesis (e.g., VEGF, FGF, thrombospondin, BAI-1, GDAIF) and MCC.

c. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., BclXL, BclW, Mcl-1, A1, Bfl-1) or counteract Bcl-2 function and promote cell death (e.g., Bax, Bak, Bik, Bim, Bid, Bad, Harakiri).

4. Other agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adehesion, or agents that increase the sensitivity of the endothelial cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the present invention by establishment of an autocrine or paracrine effect on endothelial cells. Increases intercellular signaling by elevating the number of GAP junctions would increase the anti-hyperproliferative effects on the neighboring endothelial cell population. In other embodiments, cytostatic or differentiation agents can be used in combination with the present invention to improve the anti-hyerproliferative efficacy of the treatments. Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a endothelial cell to apoptosis, such as the antibody c225, could be used in combination with the present invention to improve the treatment efficacy.

### H. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

1. Materials and Methods

a. Animals

3-6 wk-old female/male BALB/c nude mice were purchased from Harlan Inc. (Indianapolis, Ind.). Animals were housed in specific pathogen-free units of the Department of Veterinary Medicine and Surgery at M. D. Anderson Cancer Center, Houston, Tex.

b. Virus

Control adenovirus (Ad-c) was prepared by deletion of E1 region from adenovirus serotype 5. Adenovirus containing human extracellular mda-7 (Ad-mda7EC) was constructed by Introgen Therapeutics Inc., Houston, Tex.

c. Cell preparation and infection with adenovirus

All the cell lines are obtained from the American Type Culture Collection (ATTC, Rockville, Md.). The cells were grown in DMEM medium (GIBCO/BRL, Life Technologies, Grand island, N.Y.) with 100 IU/ml penicillin, 0.1 mg/mL streptomycin and 10% fetal calf serum, HyClone, Logan, Utah), according to ATCC's recommendation. The cells were tested and verified to be free of mycoplasma and used in the log phase of growth. Cells were routinely harvested with 0.125% Trypsin-1.3 mM EDTA (GIBCO).

d. In Vitro Transfection

Cells were plated at a density of 5×105 cells per 60 mm2 in RPMI/10% FBS media and grown in 5% CO2 at 37° C.

e. Recombinant Adenovirus Production

Replication deficient human type 5 Adenovirus (Ad5) carrying the nucleic acid encoding extracellular human mda-7 (or Luciferase gene) linked to an internal CMVIE promoter and followed by SV40 polyadenylation (pA) signal were constructed. A third control vector with just the CMV-pA construct also was constructed. The Ad-5 vectors harboring the gene cassettes were co-transfected with plasmid pJM17 (Graham and Prevec 1992) in 293 cells to rescue recombinant viruses Ad-mda7, AdLuc and AdCMVpA. Plaques were picked, virus stocks were grown and their genomes were confirmed as correct by PCR/restriction analysis and sequencing. Viruses were propagated in 293 cells and purified by HPLC.

f. Transduction and Cell Proliferation Studies

Cancer or normal cell lines used in this study are infected with Ad-mda7 (with either AdCMVpA or AdLuc as controls) in increasing MOIs (viral particles/cell; 0, 100, 250, 500, 1000, 2500, 5000, 10000 vp/cell increasing concentrations). Cells were either plated at 500-2000 cells/well in 96-well format for Tritiated thymidine incoporation-Cell Proliferation Assay or plated at 10⁵-10⁶ cells/well in a 6 well plate for protein expression or Apoptosis assays or plated at 10⁴ cells/well for Alamar-blue assay.

For infection Ad-mda7 or AdLuc (or AdCMVpA) were used at increasing MOIs (based on viral particles/cell; MOI ranged from 0-10,000 viral particles/cell). For tritiated thymidne/apoptosis and protein expression and alomar assays, cells were analyzed 3 and 5 days post-infection

g. Tritiated Thymidine Assay

Growth inhibition of cells after treatment is measured by analysis of DNA-synthesis. Briefly, for the ³H-thymidine incorporation assay cells will be plated at 200-5000 cell per well in a 96-well format and grown in DMEM/10% FBS in a 5% CO2 incubator at 37° C. overnight. The next day the media is aspirated and replace with 50 ml DMEM/10% FBS containing the appropriate adenovirus at the appropriate MOI. Cells will be incubated with infecting media for 1 to 4 hrs and then diluted to 200 ml total volume and grown overnight. Media is replaced with DMEM/10% FBS/mCi ³H-thymidine and grown for 16 to 18 hrs. Stock solution of 100 uCi/mL of H3-thymidine (Amersham) is prepared by dilution into high glucose DMEM (GIBCO). ³H-Thymidine was added to each well at a final concentration of 1 μCi/mL. The reaction is stopped 15 hours later by removal of the supernatant from recipient cells. The cells were harvested by addition of 100× Trypsin/EDTA (GIBCO) to each well for 15 minutes at 37° C. Cells were collected on a filter in the 96-well format using a Packard Filtermate Cell Harvester following manufacturer's protocol and washed in deionized water and methanol. The filter were dried and analyzed in Matrix 9600 (Packard) and cell proliferation using Viral Particles/cell against Tritiated Thymidine uptake counts were plotted.

h. Alamar Blue Assay

Growth inhibition of cells also was measured by Alomar Blue Assay. Briefly, cells were plated at 10⁴ cells/well density in a 96-well plate format. Four days after infection with different MOIs of Ad-mda7 or control vectors (as previously described), 20 μL of alamar blue dye was added to each well and the plate was incubated at 37° C. for 6-8 hours. The plates are then read for optical density on the Dynatech MRX plate reader at wavelength of 595 nm. Revelation 3.2 software program was used to plot MOIs against optical density values at 595 nm.

i. TUNEL Assay

Cancer cells were seeded in Lab-Tek chamber slides (Nunc) at density of 10⁴ cells/chamber. Cells were transduced with desired concentration of Advectors. At different day points, post-infection, cells were analyzed according to manufacturer's instruction for Apoptosis using the Chromogenic TUNEL-peroxidase assay (“In Situ Death Detection Kit, POD”, Boehringer Mannheim).

j. Annexin V Assay

Cancer cells were also analyzed for Apoptosis, post-Ad-mda7 treatment, by ApoAlert Annexin V-FITC kit (CLONTECH). After induction of apoptosis in cells, phosphatidylserine (PS), which is predominantly located on the inner leaflet of the plasma membrane, is rapidly translocated to the outer leaflet via a flippase mechanism. In the presence of Ca2⁺, annexin V binds PS with high affinity and FITC conjugated to Annexin help to pinpoint apoptotic cells both via flourescent microscopy and FACs analysis.

k. DNA Staining with Propidium Iodide (PI)

For determiniNg cells at different stages of cell cycle, Ad-mda7 infected Cancer cells were prepared as a single cell suspension of 1-2×10⁶ cells/mL of PBS. After the cells are fixed with cold 70% ethanol for 2 hours, the cells are centrifuged, and the fixative decanted, and washed 2× with PBS and then stained with Propidium Iodide working solution which included PI at 50 μg/mL and RNAse at 20 μg/mL in PBS. Treated cells were then analyzed by FACS.

1. Tumor Xenograft Models

Tumor cells are plated at a density of approximately 20-40% confluency in 150 mm² dishes in RPMI/10% FBS media supplemented with penicillin, streptomycin and fungizone, and grown in 5% CO₂ at 37° C. until approximately 80% confluent. Cells are washed twice in PBS, trypsinized, and counted. Cells are diluted to a concentration of 5×10⁶ cells/100 ml in PBS. BALB/c nude mice will be injected subcutaneously with 5×10⁶ tumor cells in 100 ml of PBS.

m. Tube formation assay

Human umbilical vein endothelial cells (HUVECs; Clonetics) were seeded on 1% gelatin-coated plates and incubated at 37° C. for 24 hours. After incubation, cells were infected for 1 hour with Ad-luc or Ad-mda7 at 10,000 vp/cell in serum-free medium. Cells exposed to medium alone served as negative controls while cells exposed to Suramin (50 μM) served as positive controls. After a 48-hour incubation period (37° C. in serum-containing medium), infected cells were harvested, counted, and added to Matrigel-coated 24-well plates in triplicate (1.2×10⁵ cells per well). Twenty-four hours later, cells were fixed with 10% buffered formalin and examined for differentiation (tube formation) by using an Olympus IX-70 inverted bright-field microscope at 4× and 10× magnification. cl Example 1

### AD-MDA7 Kills Cancer Cells and Induces Apoptosis

1. Breast Cancer Cells

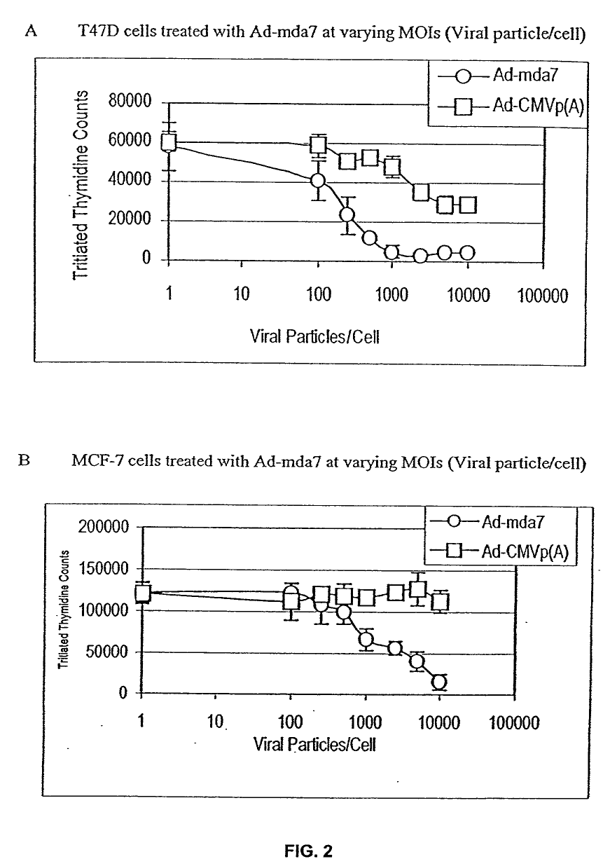

A series of breast cancer cell lines (T47D, MCF-7, BT-20, MDA-MB-361, SKBr3, MDA-MB-231, MDA-MB-468) were transduced with Ad-mda7 (or Ad-CMVp(A) or Ad-luc as control vectors). The cell lines were strongly growth-inhibited by Ad-mda7 transduction. The two cell lines that demonstrated the highest sensitivity to Admda7 were T47D (p53 mutated) and MCF-7 (p53 wild-type) (FIGS. 2A and 2B), as determined by ³H-thymidine incorporation assay. Cancer cells were analyzed 3-6 days after Ad-mda7 transduction. See Table 3 below.

TABLE 3
Summary of breast cancer lines used for Ad-mda7 studies.

| Cell Line | Tumor type | p53 status | Source |
|---|---|---|---|
| Breast Cancer | | | |
| (1) T47D | ductal carcinoma | L194F | ATCC |
| (2) MCF-7 | carcinoma | wt | ATCC |
| (3) MDA-MB-361 | adenocarcinoma | wt | ATCC |
| (4) MDA-MB-231 | adenocarcinoma | R280K | ATCC |
| (5) MDA-MB-468 | adenocarcinoma | R273H | ATCC |
| (6) SKBr-3 | adenocarcinoma | Mut | ATCC |
| (7) BT-20 | carcinoma | Mut | ATCC |
| Normal | | | |
| (1) MJ90 | fibroblast | wt | Smith lab |
| (2) HUVECs | endothelium | wt | Clonetics |
| (3) HMECs | mamm. epithelium | wt | Clonetics |

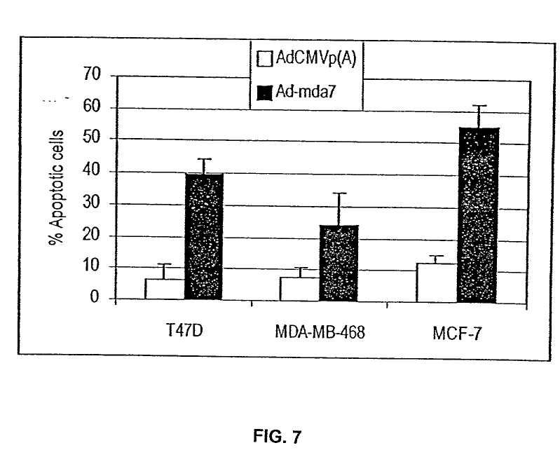

FIG. 7 illustrates the high levels of apoptosis (as measured by Annexin V staining) induced in breast cancer cell lines by Ad-mda-7. Annexin V staining identifies cells in early and mid-stages of apoptosis, whereas the TUNEL assay detects DNA cleavage products, one of the final stages of apoptosis. TUNEL assays performed on MCF-7 cells infected with Ad-mda7 confirmed that these cells are killed via apoptotic pathways. Ad-CMVp(A) or Ad-luc control vectors were ineffective at inducing apoptosis.

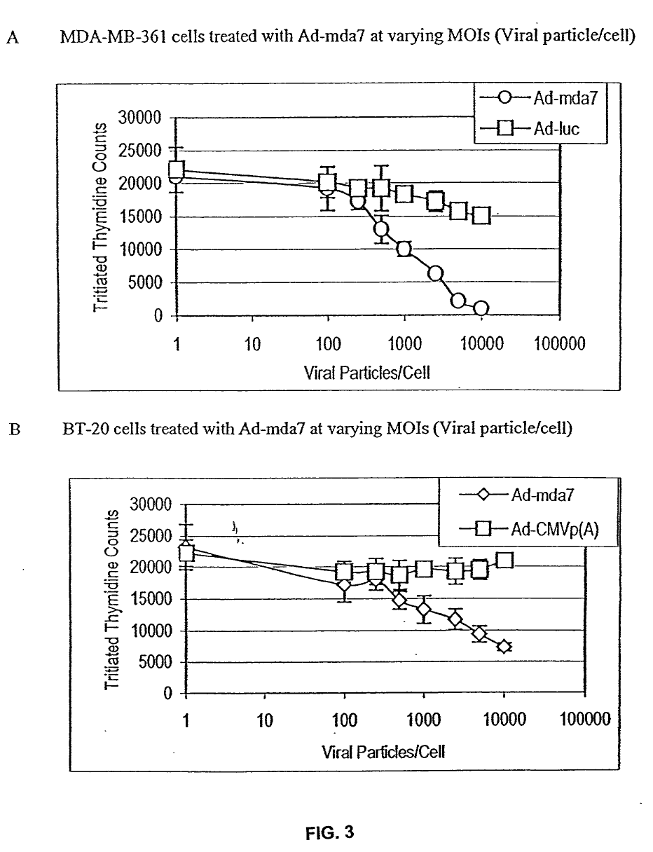

The two cell lines that demonstrated the highest sensitivity to Ad-mda7 were T47D (p53-mutant) and MCF-7 (p53 wild-type) (FIG. 2A and 2B). The Ad-mda7 concentration needed to inhibit growth by 50% (IC₅₀) of the T47D or MCF-7 cells averaged 500 and 1500 vp/cell, respectively (Table 4). Also included in FIG. 3 (Panels A and B) are representative experiments using MDA-MB-361 and BT-20 cells. These two cell lines also showed marked sensitivity to Ad-mda7 infection. Table 4 summarizes the responsiveness of breast cancer cells to Ad-mda7 infection (as determined by a comparison of IC₅₀ values for Ad-mda7 and control Ad vector). Also included in Table 4 are the IC₅₀ values in normal cell lines.

TABLE 4
Summary of IC₅₀ values of Ad-mda7 in Breast Cancer and Normal lines

| | | IC₅₀ range | |
|---|---|---|---|
| Cell Line | Tumor type | Admda-7 | Control* |
| Breast Cancer | | | |
| (1) T47D | ductal carcinoma | 150-500 | >10,000 |
| (2) MCF-7 | carcinoma | 1200-4000 | >10,000 |
| (3) MDA-MB-361 | adenocarcinoma | ˜1500 | >10,000 |
| (4) MDA-MB-231 | adenocarcinoma | ˜3000 | >10,000 |
| (5) MDA-MB-468 | adenocarcinoma | >10,000 | >10,000 |
| (6) SKBr-3 | adenocarcinoma | ˜5000 | >10,000 |
| (7) BT-20 | carcinoma | ˜2500 | >10,000 |
| Normal | | | |
| (8) MJ90 | fibroblasts | >10,000 | >10,000 |
| (9) HUVECs | endothelium | >10,000 | >10,000 |
| (10) HMECs | mamm. epithelium | >10,000 | >10,000 |

*The control vectors used in these experiments were either Ad-CMVp(A) or Ad-luc.

2. Ad-mda7 Kills Lung Cancer Cells and Induces Apoptosis

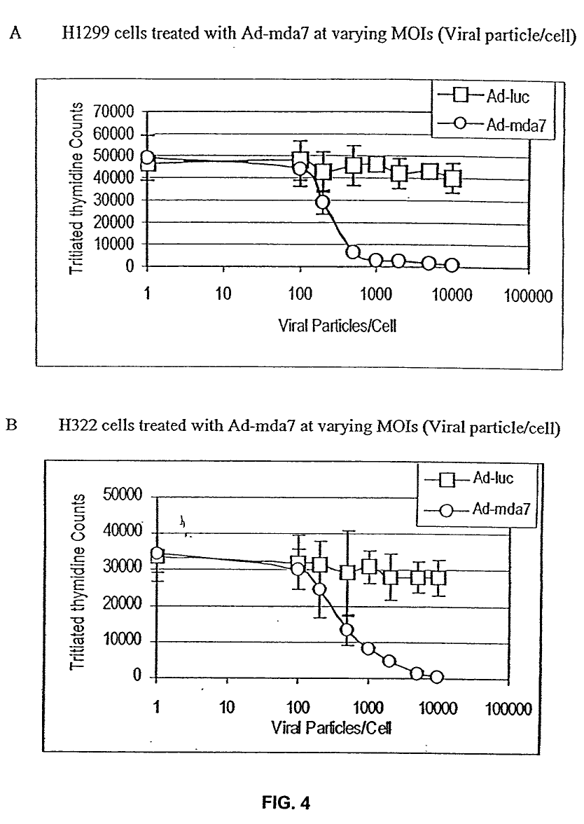

Six lung cancer lines (H1299, H460, A549, H322, H358 and SaosLM2) were infected with Ad-mda7. All of these demonstrated effective killing by Ad-mda7 transduction. The H1299, and H322 cell lines were the most sensitive to Ad-mda7 killing (see FIG. 4A and B). The IC₅₀ in these lines ranged from 600 vp/cell to 2000 vp/cell as determined by ³H-thymidine incorporation assay.

3. Ad-mda7 Kills Colorectal Cancer Cells and Induces Apoptosis

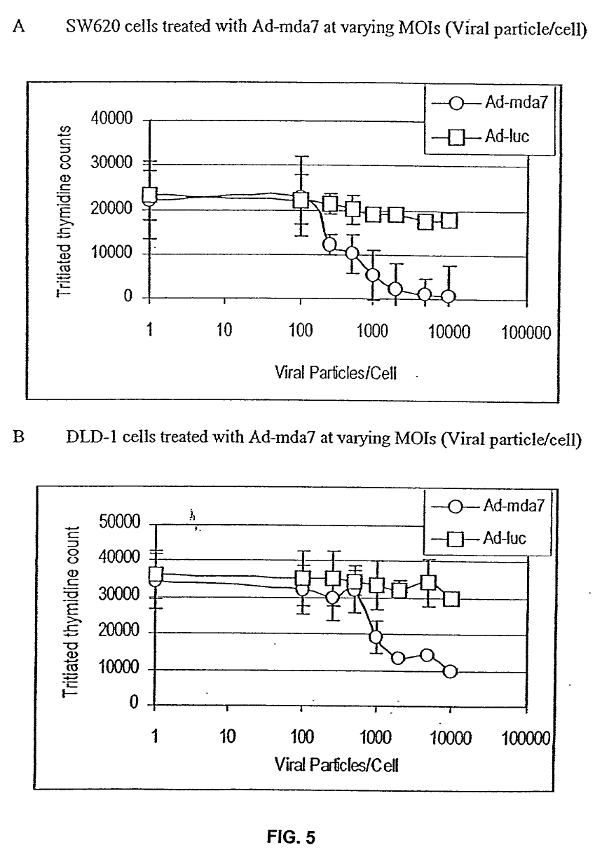

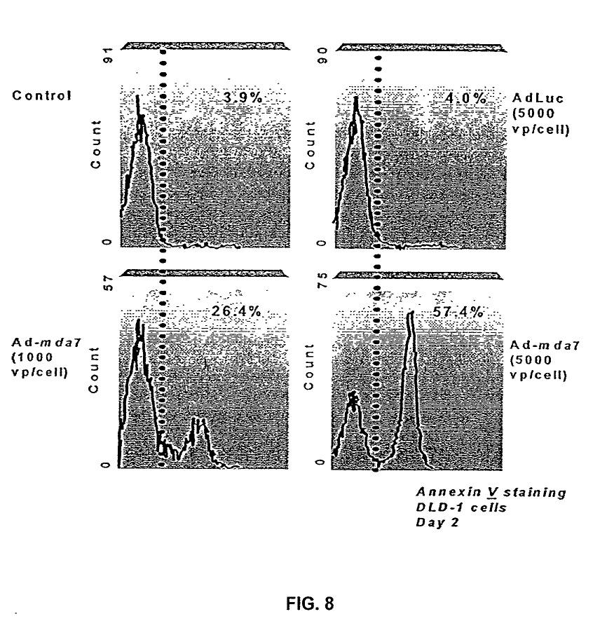

Six colorectal cancer lines (DLD-1, SW-620, SW-480, HT-29, HCT-116, LS174T) were infected with Ad-mda7. All of these cell lines were effectively growth inhibited by Ad-mda7 transduction, with SW620, DLD-1 and SW-480 being the most sensitive. SW620 cells treated with Ad-mda7 at variying MOIs is shown in FIG. 5A, while DLD-1 cells are shown in FIG. 5B. Cell proliferation, as determined by ³H-thymidine incorporation assay, demonstrated an IC₅₀ that averaged 1000 vp/cell in the more sensitive cell lines to 2000 vp/cell in the other less-sensitive cell lines. The DLD-1 cell line was infected with Ad-mda7 at 1000 and 5000 vp/cell, using uninfected cells and Ad-Luc as controls. Forty-eight hours later the transduced cells were analyzed for apoptosis using Annexin V staining in conjunction with FACS analysis. Neither the uninfected or AdLuc-infected (5000 vp/cell) cells showed signs of apoptosis, whereas Ad-mda7 infected cells exhibited approximately 26% apoptotic cells at 1000 vp/cell and 58% apoptotic cells at 5000 vp/cell (FIG. 8).

4. Ad-mda7 Infection in Normal Cells

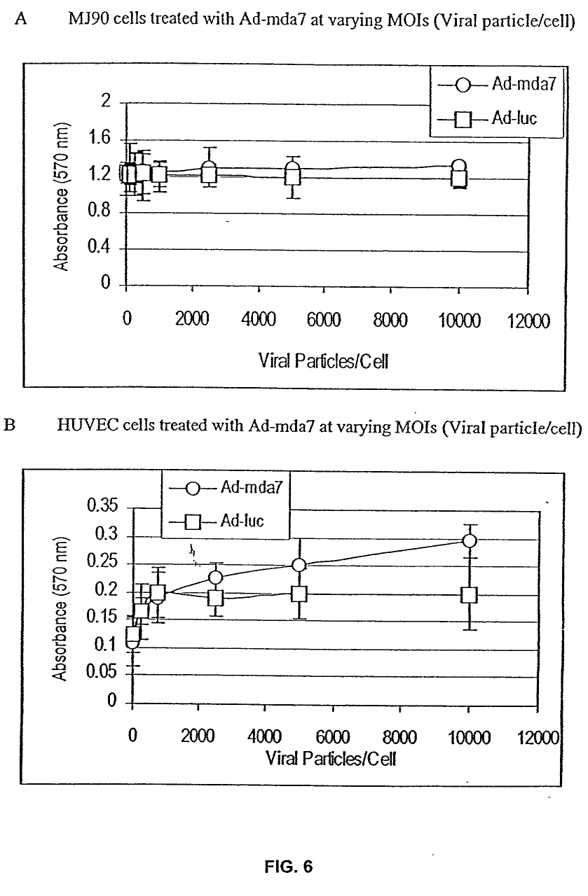

Three normal human cell lines (MJ90 fibroblasts, HUVEC endothelial cells and human mammary epithelial cells) showed no growth inhibition when infected with Ad-mda7. The primary fibroblast cell line MJ90 showed overlapping growth curves when treated with Ad-mda7 or Ad-luc control vector (FIG. 6A). HUVEC and human mammary epithelium cells showed similar results (FIG. 6B).

5. Protein Analyses

Cell lysates obtained from Ad-mda7 transduced cancer cell lines were size fractionated by SDS-PAGE followed by western-blot analysis using a rabbit anti-MDA7 antibody. The migration of the MDA-7 protein was consistent with an approximate size of 23 kD, however, an additional band at 17 kD was also observed. A Western blot analysis of H1299 (lung cancer) and DLD-1 (colorectal cancer) cell lines was performed after Ad-mda7 and Ad-luc infection. Two bands at approximately 23 and 17 kD were observed. Similar molecular weight size bands were also seen in breast cancer lines infected with Ad-mda7. During the first 48 hours post-infection the 17 kD band was the major species observed in DLD-1 cells. At 72 and 96 hours post infection, the intensity of the 23 kD band decreased with time and other smaller degradation products were seen. In H1299 cells, both bands had similar intensities. The blots were also probed for β-actin, and at 72 and 96 hours post-infection, actin was substantially degraded (data not shown), consistent with the rapid apoptotic death of cells.

As seen in these protein expression studies, lysates from Ad-mda7 infected cells show a 23 kD/17 kD doublet, suggesting that MDA-7 is processed intracellularly. Previous studies by Su et al. (1998) indicated that in human melanoma cells induced with interferon β and mezerin, the 23 kD MDA-7 protein translocated from the cytosol to the nucleus. On the basis of primary protein sequence analysis, MDA-7 does not possess any consensus nuclear localization motifs, which may suggest MDA-7 protein associating with a cytoplasmic chaperone (such as HMC) (Jiang et al., 1995; 1996). It was proposed that this association may facilitate the translocation of mda-7 into the nucleus.

6. Apoptosis Studies

Annexin V staining identifies cells in early and mid-stages of apoptosis, whereas the TUNEL assay detects DNA cleavage products, one of the final stages of apoptosis. FIG. 7 illustrates the high levels of apoptosis (as measured by Annexin V staining) induced in breast cancer cell lines by Ad-mda7. TUNEL assays were performed on MCF-7 cells infected with Ad-mda7, thus confirming that these cells are killed via apoptotic pathways. Ad-CMVp(A) or Ad-luc control vectors were ineffective at inducing apoptosis.

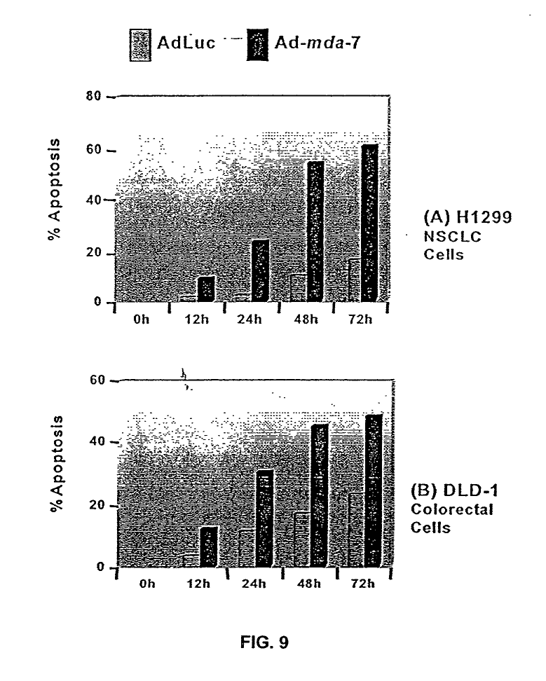

Further examples of Ad-mda7-induced apoptosis are shown in FIG. 9. The DLD-1 cell line was infected with Ad-mda7 at 1000 and 5000 vp/cell, using uninfected cells and Ad-Luc as controls. Forty-eight hours later the transduced cells were analyzed for apoptosis using Annexin V staining in conjunction with FACS analysis (FIG. 8). Neither the uninfected or Ad-Luc infected (5000 vp/cell) cells showed signs of apoptosis, whereas Ad-mda7 infected cells exhibited approximately 26% apoptotic cells at 1000 vp/cell and 58% apoptotic cells at 5000 vp/cell. Ad-mda7 caused rapid induction of apoptosis (FIG. 9). Two cell lines representing NSCLC and colorectal cancer are shown. Substantial levels of apoptosis were evident as soon as 12 hours post-infection with Ad-mda7, and increased over the next few days. The demonstration of apoptosis as soon as 12 hr post-infection is notable as immunoreactive MDA-7 protein is just detectable at 12 hr and, generally, does not peak until 24-48 hr post-infection. Ad-p53 can also cause rapid induction of apoptosis, however, other tumor suppressors, such as p16 or PTEN tend to cause apoptosis only after 2-3 days post infection with the Ad expression vector.

7. Ad-mda7 Increases Bax Protein Levels in Lung, Breast and Colorectal Cancer Lines

Regulation of programmed cell death relies on the interaction between signaling pathways that either promote or inhibit apoptosis (Reed, 1997; White, 1996). The bcl-2 family members (bcl-2, bcl-w, bax, bad, bak, bcl-xs) play an important role in apoptotic signaling (Sedlak et al., 1995; Reed et al., 1996). Using Western blot analysis in conjunction with an anti-bax antibody it was determined that Ad-mda7 infection upregulated the BAX protein in T47D, DLD-1, A549 and H460 cells. Western blot analysis of lysates prepared 24 hours after infection with 30 to 150 pfu/cell of Ad-mda7 demonstrated increased expression of BAX in all cell lines tested. For example, upregulation of BAX in Ad-mda7 infected T47D cancer cell line was observed by Western blot analysis. Cells were infected with Ad-mda7 and analyzed for MDA-7 and BAX protein expression. Ad-mda7 increased BAX expression in T47D, as was observed with the other cell lines.

8. Endogenous Expression of Mda-7 in Cancer and Normal Cells

Of the more than 50 tumor cell lines evaluated for endogenous Mda-7 protein expression, only two, DLD-1 (colorectal) and LnCap (prostate) were weakly positive. Studies are underway to look at mda-7 mRNA in the various cancer lines. Table 5 is a list of some of the cancer lines used in the Ad-mda7 studies and their endogenous MDA-7 status. There was no correlation between the anti-tumor activity of Ad-mda7 and MDA-7 endogenous expression based on Western blot analysis (Table 5).

TABLE 5

| | Endogenous MDA-7 | |
|---|---|---|
| Cell Type | protein | Ad-mda7 killing |
| (A) Normal lines | | |
| (1) MJ90 | −− | −− |
| (2) HUVEC | −− | −− |
| (3) HMEC | −− | −− |
| (B) Breast cancer lines | | |
| (1) T47D | −− | ++++ |
| (2) MCF-7 | −− | +++ |
| (3) MDA-MB231 | −− | ++ |
| (4) MDA-MB468 | −− | ++ |
| (C) Lung cancer lines | | |
| (1) H1299 | −− | ++++ |
| (2) A549 | −− | ++ |
| (3) H460 | −− | ++ |
| (D) Colorectal cancer lines | | |
| (1) DLD-1 | ++ | +++ |
| (2) SW620 | −− | +++ |
| (3) HCT116 | −− | ++ |
| (4) HT29 | −− | ++ |
| (E) Prostate cancer lines | | |
| (1) LnCap | ++ | +++ |
| (2) Du145 | −− | ++ |

Note:
−− denotes undetectable endogenous protein/no response to Ad-mda7 infection;
++ denotes presence of endogenous mda7 protein or effective responsiveness to Ad-mda7.

9. Ad-mda7 Functions Independently of Endogenous p53, Rb, Ras, and p16 Status

Table 6 presents the status of different tumor suppressor/oncogene/cell cycle regulating genes and their response to Ad-mda7 infection in different cell lines used in this study. The growth-inhibitory action of MDA-7 was observed in a wide variety of cancer cell lines, independent of their p53, RB, p16, and Ras status. Although, Bax expression is positively regulated by wild-type p53 (Han et al., 1996), the ability of MDA-7 to induce BAX appears to be independent of p53 since BAX up-regulation is observed in p53-mutant (DLD-1, T47D) and p53-wild-type (H460). It is interesting to note that MDA-7 was able to effectively induce apoptosis in the MCF-7 breast cancer cells that are devoid of caspase 3, one of the several caspases involved in the down-stream apoptotic events.

TABLE 6

| Cell Type | Ad-mda7 effect | p53 | RB | ras | p16 |
|---|---|---|---|---|---|
| (A) Normal lines | | | | | |
| (1) Melanocytes | ND | wt | wt | wt | wt |
| (2) MJ90 fibroblasts | −− | wt | wt | wt | wt |
| (3) HUVEC | −− | wt | wt | wt | wt |
| (4) HMEC | −− | wt | wt | wt | wt |
| (B) Cancer Lines | | | | | |
| (1) T47D | ++++ | mut | −− | wt | −− |
| (2) MCF-7 | +++ | wt | −− | wt | −− |
| (3) H1299 | ++++ | null | wt | mut | −− |
| (4) Saos-LM2 | ++ | del | −− | wt | del |
| (5) A549 | ++ | wt | −− | mut | −− |
| (6) H460 | ++ | wt | wt | mut | del |
| (7) SW620 | +++ | mut | −− | mut | −− |
| (8) HCT116 | ++ | wt | −− | mut | −− |

Note:
ND, Not determined; mut, mutation; del, deletion; wt, wild-type

### Example 2

### MDA-7 Cellular Localization Studies

1. Surface Expression Studies

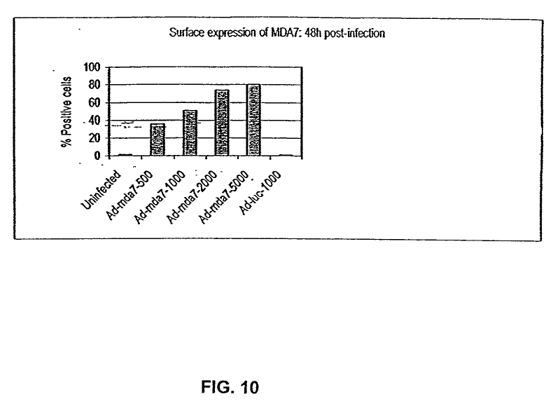

The H460 NSCLC cell line was treated with increasing MOIs of Ad-mda7 or Ad-luc as control, and 48 h later, the cells were stained with the polyclonal anti-MDA-7 antibody and analyzed by FACS analysis (FIG. 10). A high level of staining was observed in the Ad-mda7 treated cells only. The staining was dose-dependent and approximately 50% of cells were MDA-7 positive at 1000 vp/cell. This result indicated that Ad-mda7 treatment of H460 cells resulted in high levels of protein production (verified by Western blot analysis) and that the protein appeared to on the cell surface.

2. Confocal Microscopy Studies

To confirm and extend the results shown in FIG. 10, confocal microscopic analyses were performed on various cell lines (H460, H1299, T47D and DLD-1 cells) to determine sub-cellular distribution MDA-7 protein after Ad-mda7 treatment. Background staining in untreated or Ad-luc-treated cells was low and diffuse. The background is believed to be due to the anti-MDA-7 reagent being a polyclonal antiserum. However, highly specific staining was observed when cells were treated with Ad-mda7. At low MOIs, distinct membrane staining was observed with punctate staining in the cytoplasm. At higher MOIs, the punctate staining and membrane staining were reproduced and more intense. The pattern of staining was suggestive of a secreted protein, with the punctate staining representing protein trafficking and release at the plasma membrane. Similar observations were observed in the other cell lines

In additional confocal microscopy experiments, cancer cell lines were treated with Ad-mda7 and analyzed for apoptosis (Annexin V staining), DNA content (Hoechst), Ca2+ influx/eflux (Fluo 3, Molecular Probes) and mitochondrial integrity (MitoTrack, Molecular Probes). The protocols used were those established in the Confocal Microscope Facility, UTHSC, Houston, Tex.

Confocal microscopic studies of H460 and MCF-7 cells were done. They show a composite of individual microscopic fields: (1) denotes surface expression of MDA-7 (red surface and punctate staining), (2) showing apoptosing cells (polarized green staining), (3) Hoechst staining to identify nuclei (blue) and (4) composite of (1) (2) and (3).

Calcium and mitochondrial staining was done in Ad-mda7- or Ad-luc control-transduced cells. Cells were plated on laminin-coated cover-slips and treated with FLUO-3 (for Ca²⁺) or with Mitotracker (for mitochondria). The control Ad-luc treated cells show a well distributed intracellular calcium content (green fluorescence) and displayed good mitochondrial integrity (red staining). However, on Ad-mda7 treatment, intracellular Ca²⁺ levels are disrupted and the mitochondrial integrity is disrupted.

### Example 3

### Secreted MDA-7 Protein

1. Secretion of MDA-7

H1299 cells were infected with Ad-mda7 (MOI of 1000 vp/cell) for 6 hours, washed with fresh media and incubated at 37° C. in fresh DMEM media. Twenty-four hours later, the cell lysate and the growth media were analyzed for MDA-7 protein expression using Western blot. Ad-mda7 transduced cells showed a specific 40 kD protein produced in growth media, which was absent in untransduced or Ad-luc transduced cells that only showed 19 kD and 23 kD bands. A dose-dependent increase in the intra-cellular MDA-7 and the extra-cellular MDA-7 protein was observed. As a control, the blot in Panel B was probed with an anti-actin antibody. As predicted, the cell lysates showed an actin signal at approximately 40 kD, whereas the cell supernatants did not show any actin signal. This suggests that the MDA-7 protein signal observed in the supernatants is due to active release/secretion of MDA-7 and is not due to release from dying cells.

2. Glycosylation of Secreted MDA-7 Protein

The supernatant from Ad-mda7 transduced H1299 cells was a good source of obtaining the secreted MDA-7 protein. The supernatant was further evaluated for protein glycosylation. Supernantant was treated with the following three enzymes either individually or in different combinations. The enzymes used were sialidase (neuraminidase), endoglycosidase-H and endoglycosidase-F (all obtained from Sigma). The samples were analyzed by Western blot using the specific anti-MDA-7 rabbit polyclonal antibody.

Endoglycosidase treatment suggests that soluble MDA-7 protein is glycosylated. Using various glycosidases, especially Endo F, a lower molecular weight band is also observed (which is approximately the same size as the MDA-7 protein band observed in cell lysate.

3. Inhibition of Glycosylation and Secretion of MDA-7 Protein

Two antibiotics, Tunicamycin and Brefeldin A, have been used to provide a more detailed characterization of the secretion of soluble MDA-7. N-linked glycosylation plays an important role in a protein's ultimate processing, whether it is sorted to a lysosomal pathway, or translocated to the cell surface or secreted. Using Tunicamycin, the N-linked glycosylation process in the golgi apparatus can be inhibited, thus inhibiting protein secretion or other sugar-dependent sorting processes. Brefeldin A is a fungal metabolite (macrocyclic lactone) which exhibits a wide variety of antibiotic activities. Brefeldin A reversibly inhibits the intracellular translocation of proteins (during transport of protein to the cell surface for secretion or expression. Both Tunicamycin and Brefeldin A effectively inhibit the secretion of soluble MDA-7 protein. Therefore, intracellular processing and glycosylation appear to be required for MDA-7 secretion.

4. Secreted MDA-7 Protein Induces Killing in Cancer Cells

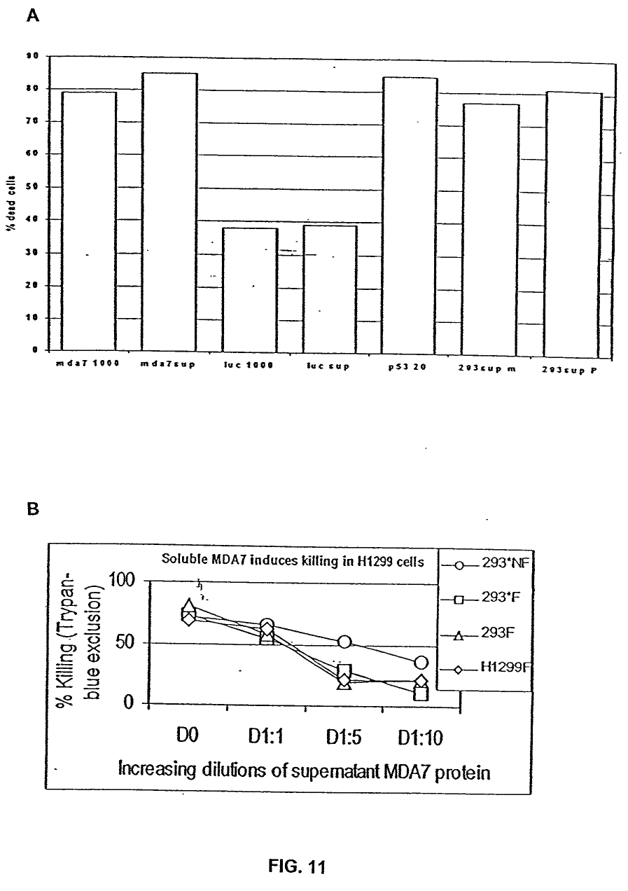

The secreted form of MDA-7 (sMDA-7) was produced using various cell lines and evaluated for anti-tumor activity. A representative experiment is shown in FIGS. 11A and 11B. Soluble MDA-7 was analyzed for its anti-proliferative effects on H1299 cells. Briefly, H1299 cells were plated at cell density of 10³ cells/chamber in Nunc chamber slides. 24 hours later, the cells were challenged with supernatants obtained from H1299 cells transduced with either Ad-mda-7 or Ad-luc (at 1000 vp/cell infection). Ad-mda7 and Ad-luc viruses were also used as additional controls. The soluble protein supernatants (500 uL total volume, different dilutions) were applied to naive H1299 cells and 24 hours later an additional 0.5 mL of 10% FBS in DMEM was added. After 24 and 48 hours, the cells were microscopically examined for viability using the trypan blue exclusion staining. The soluble MDA-7 protein showed H1299 killing after 48 hours; however, Ad-luc supernatants had little effect (FIG. 11A).

Various dilutions of soluble MDA-7 supernatant were also analyzed for H1299 killing using the Trypan blue exclusion assay. A concentration-dependent bystander killing effect of soluble MDA-7 was observed (FIG. 11B).

### Example 4

### Combination Studies of Ad-mda7 in Breast Cancer Lines

1. Combination with Tamoxifen

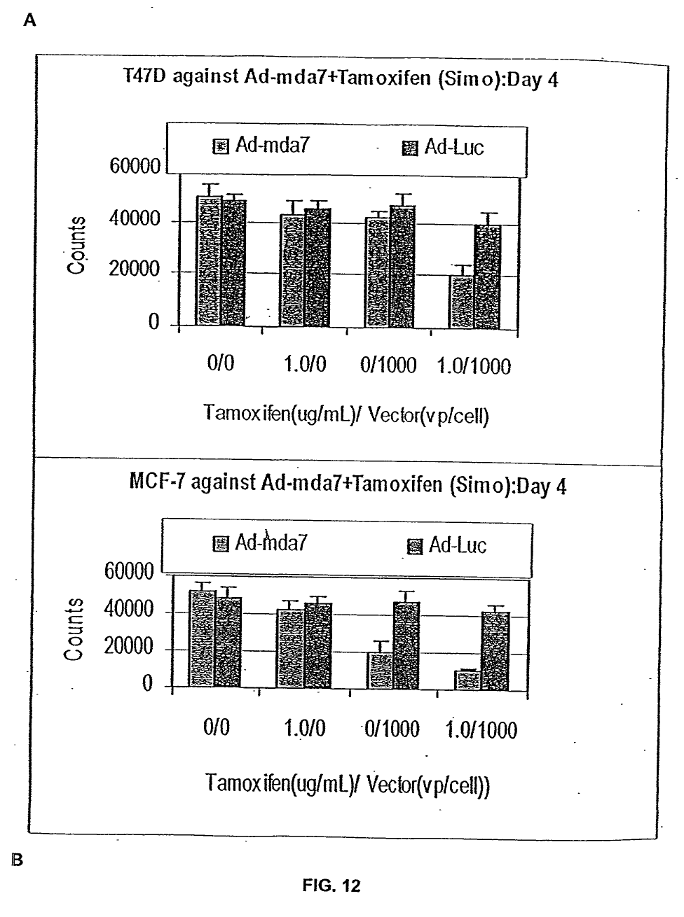

Ad-mda7 has been combined with tamoxifen and evaluated for anti-tumor effects in breast cancer cell lines (FIGS. 12A and B). The graphs demonstrate that combining these two agents provides superior anti-tumor activity compared to either agent alone. The effect of tamoxifen on T47D cells is shown (FIG. 12A) and on MCF-7 cells (FIG. 12B). Cells were plated and four days after treatment, a tritiated thymidine assay was performed to measure DNA replication. Cells were treated with 0/0 (no drug and no vector) or varying doses of tamoxifen or vectors (Ad-luc or Ad-mda7). In T47D cells, tamoxifen or Ad-mda7 had minimal effect on DNA replication. However, when the tamoxifen and Ad-mda7 were combined, a supra-additive effect was observed. In MCF-7 cells, tamoxifen had little effect at 1 ng/ml dose. Ad-mda7 reduced signal compared to Ad-luc. However when tamoxifen was combined with Ad-mda7, a supra-additive effect was observed, again demonstrating the enhanced effects of combining a chemotherapeutic agent with Ad-mda7.

2. Combination with Adriamycin

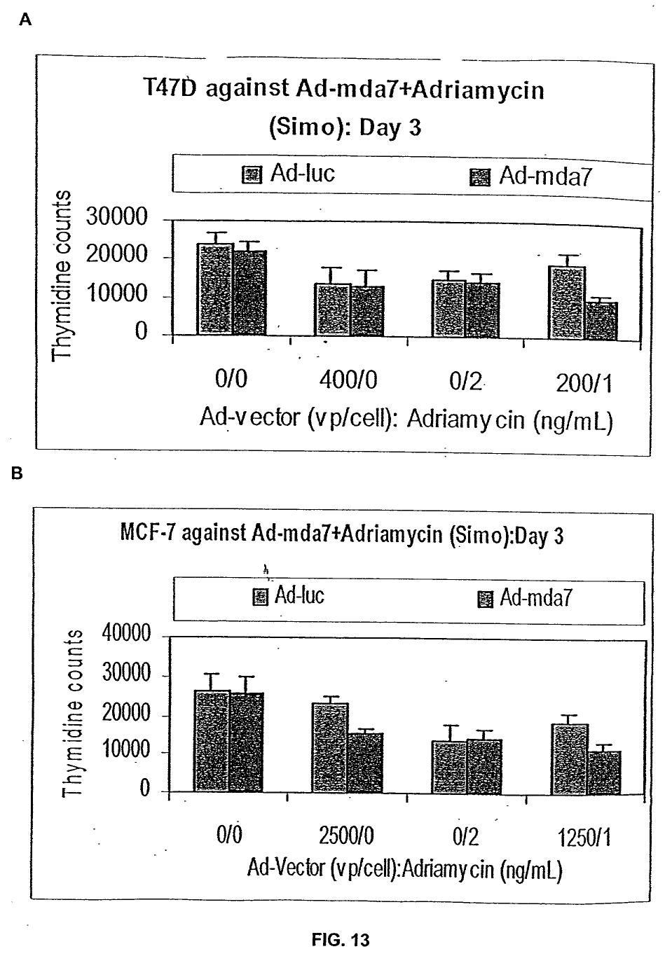

Ad-mda7 has been combined with adriamycin and evaluated for anti-tumor effects in breast cancer cell lines (FIGS. 13A and B). The graphs demonstrate that combining these two agents provides superior anti-tumor activity compared to either agent alone.

### Example 5

### Activation of Caspase Cascade by AD-MDA7

1. Material and Methods a. Cell Culture

Human non-small cell lung carcinoma cells A549, H460, H1299, human prostate cancer cells DU145, and human breast cancer cells MCF-7 were obtained from the American Type Culture Collection (ATCC, Bethesda, Md.). All cells were maintained in DMEM medium containing 10% of Fetal Bovine Serum, antibiotics and L-glutamine. Normal human bronchial epithelium cells (NHBE cells) were obtained from Clonetics Inc (Clonetics Inc., Walkersville, Md.) and maintained according to the manufacturer's instructions.

The cells were verified to be free of mycoplasma and used in the log phase of growth. Cells were routinely harvested with 0.125% Trypsin-1.3 mM EDTA (GIBCO).

b. Construction of Recombinant Adenoviral Vector

Same as described above.

c. Determination of Cell Growth Rate

Cancer or normal cell lines used in this study were plated in 12-well dishes with 2×10⁴ cells in each well. Cells were infected with Ad-mda7, with Ad-Luc controls (5000 viral particles/cell), or with PBS as an additional control. Cells were harvested by trypsinization, diluted with trypan blue (GIBCO) and the numbers of viable cells were counted on a hemocytometer. In addition, inhibition of cell growth was assayed by XTT assay as per the manufacturer's guidelines (Cell Proliferation Detection Kit II, Roche) or by H³-thymidine assay.

d. Cell Cycle Analysis

Fluorescence-activated cell sorter analysis was performed as follows: cells (5×10⁵/plate) were seeded on 10 cm plates and infected with PBS, Ad-mda7 or Ad-Luc at 5000 vp/cell. Cells were harvested by trypsinization at designated times (24, 48, 72 hrs after infection) and washed twice with PBS. Cells were fixed with 70% ethanol, washed with PBS twice and resusupended with 500 μl of PI solution (5 μg/ml PI and 10 μg/ml RNase). Cells were analyzed using a FASCscan analyzer.

e. Detection of Apoptosis

Tumor cells were seeded in chamber slides (Falcon) at a density of 1×10⁵ cells/chamber. Cells were transduced with Ad-mda7 or Ad-Luc vectors. At different days post-infection, cells were analyzed for apoptosis by Hoechst 33342 staining (Boehringer Mannheim) and terminal deoxynucleotidyl transferase-mediated biotinylated UTP nick end labeling (TUNEL) staining with Terminal Transferase (Boehringer Mannheim).

f. Immunohistochemical Staining

Immunohistochemical staining was carried out on virus infected cells to determine MDA-7 protein expression. Briefly, cells (H1299, A549, H460, and NHBE) were plated at a density of 1×10⁵ in chamber slides (Falcon) and infected with Ad-mda7 or Ad-Luc (5000 viral particles/cell). 48 hrs later, cells were washed with PBS and fixed in 4% formalin solution for two minutes. After blocking of endogenous peroxidase activity with 0.3% H₂O₂ in methanol for 30 minutes, cells were incubated with normal goat serum for 30 minutes at room temperature. Following incubation, slides were treated with rabbit polyclonal anti-MDA-7 antibody (1:5000 dilution) for 60 minutes. After 30 minutes incubation with anti-rabbit secondary antibody (provided with ABC kit, Vector) expression of MDA-7 in cells was detected with DAB by enhancement with avidin-biotin reaction ABC kit. The slides were counterstained with hematoxylin and then mounted with Aqua-mount (Lerner Labs, Pittsburgh, Pa.). Negative controls included cells uninfected but subjected through all staining proceeded.

g. Western Blotting Analysis

Cells were harvested by trypsinization, washed with PBS and resuspended in 100 μl of lysis buffer (62.5mM Tris-Hcl, 2% SDS, 10% glycerol, 4M Urea). Cell extracts were homogenized with sonicator for 30 sec and after an hour incubation on ice, cell extracts were spun for 5 min at 14000 rpm at 4° C. Cell extracts were collected and stored in −70° C. Protein concentrations of all extracts were determined using the Bio-Rad protein determination kit (Bio-Rad). Each of 50 μg protein samples were diluted into 20 μl with lysis buffer and 5% of 2-Mercaptoethanol (Bio-Rad) and heated in a water bath at 95° C. for 5 min. Then protein extracts were separated on a 10% SDS-PAGE gel in a vertical slab gel electrophoresis cell (Bio-Rad). Proteins were transferred from gel to nitrocellulose membrane (Hybond-ECL membranes, Amarsham International, Little Chalfont, England). Proteins were blocked in a blocking solution (5% dry milk and 0.3% Tween 20 in PBS) for one hour at room temperature. Membranes were incubated with primary antibody and then horse raddish peroxidase labeled secondary antibodies followed by application of Enhanced Chemiluminescence Western Blotting Detection System (Amersham) for 30 seconds. Proteins were visualized on Amersham Hyperfilm enhanced chemiluminescence film using exposure time varying 30 seconds to 30 minutes.

2. Inhibition of Cell Proliferation by Overexpression of MDA-7

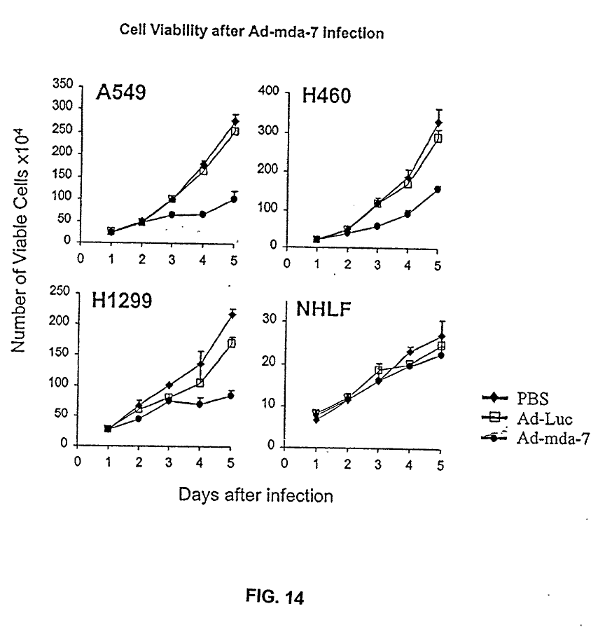

To detect MDA-7 expression in cells, A549, H1299, H460, and NHBE cells were infected with 5000 vp/cell of Ad-mda7. Forty-eight hours later cells were fixed and stained with anti-MDA-7 antibody. Uninfected cells were stained with the same antibody as controls. High level of MDA-7 expression was observed in cytoplasm of cells, while no stained cells were seen in uninfected controls (FIG. 14).

A549, H1299, H460, and NHBE cells were prepared in 12 well plates and treated with Ad-mda7, Ad-Luc, or PBS. The numbers of viable cells were counted from day 1 to day 5 after treatment. Infection with Ad-mda7 significantly suppressed cell proliferation in all the tumor cell lines as compare to PBS or Ad-Luc controls.

Cell cycle analysis using PI staining showed a G2/M cell cycle arrest in Ad-mda7-infected A549 and H1299 cells. In contrast, PBS and Ad-Luc infection did not affect the cell cycle (FIG. 14).

Following Ad-mda7 infection, morphological changeswere observed in tumor cells. These changes, such as flattenning and enlargement were observed in all of infected cell lines. Apoptotic morphological changes were visualized using Hoechst 33342. 72 hours after infection of Ad-mda7 or Ad-Luc, nuclear condensation and fragmentation were observed in Ad-mda7 infected A549, H1299, and H460 cells, while apoptotic alterations were not seen in NHBE cells. TUNEL staining demonstrated many positive cells in Ad-mda7 infected A549 cells, while very few positive cells were seen in NHBE cells. TUNEL positive cells were also very rare in Ad-luc treated samples.

These results showed significant supression of cell proliferation with concomitant G2/M cell cycle arrest and induction of apoptosis in lung cancer cell lines. In contrast, in NHBE cells overexpression of MDA-7 resulted in minimal suppression of cell proliferation, but did not induce apoptosis.

3. Upregulation of p53 and Bax in Cells with Wild type p53

Cells were infected with Ad-mda7 and Ad-Luc, and cell extracts were harvested at 24, 48, and 72 hours after infection for Western blot analysis. Cell extracts from untreated cell were harvested as a control. MDA-7 protein expression was detected in all of the Ad-mda7-infected cancer cell lines. Untreated controls and Ad-Luc-infected cells did not show any expression of MDA-7 protein. Upregulation of p53 protein was seen in p53 wild type A549 and H460 cells after Ad-mda7 infection. As predicted, no expression or modulation of p53 was seen in p53-deleted H1299 cells. An increase in BAX protein levels was demonstrated in A549 and H460 cells (p53 wild-type), while no change was observed in H1299 (p53-null) cells. The expression level of Bcl-2 was not changed in all of the three cell lines analyzed. In the Bax-deficient, human prostate cancer cell line DU145, p53 expression levels were not changed and BAX was not detected. However, DU-145 cells were sensitive to Ad-mda7 infection and displayed growth arrest and apoptosis. p53 and bax are up-regulated by Ad-mda7 in p53 wild-type tumor cells. In addition, caspases 3 and 9 and PARP are activated by Ad-mda7. Normal cells do not exhibit alterations in apoptotic mediators.

4. Activation of Caspase Cascade and Cleavage of PARP

Western blots demonstrated activation of the caspase cascade by Ad-mda7 infection. The proforms of caspase-9 and caspase-3 were cleaved and converted to the activated/ cleaved forms 48 hrs after Ad-mda7 infection in A549 and H460 cells and after 72 hrs in H1299 cells. Cleavage of caspase-8 was demonstrated after 48 hrs of Ad-mda7 infection in A549 and H460 cells. Poly (ADP-ribose) polymerase (PARP) was cleaved in A549 and H460 cells after 48 hrs in H1299 cells. In Bax-deficient DU145 cells, caspase-9 and caspase-3 were cleaved after 72 hrs of Ad-mda7 infection.

### Example 6

### IN Vivo Effects of Ad-mda7

1. Materials and Methods

a. Cell Culture

Human non-small cell lung carcinoma cells A549 and H1299 were obtained from the American Type Culture Collection (ATCC, Bethesda, Md.). All cells were maintained in RPMI1640 medium containing 10% of Fetal Bovine Serum, antibiotics and L-glutamine. Prior to start of the experiments, the cells were verified to be free of mycoplasma and used in the log phase of growth. Cells were routinely harvested with 0.125% Trypsin −1.3 mM EDTA (GIBCO).

b. Construction of Recombinant Adenoviral Vector

Replication-deficient human type 5 Adenoviral vectors (Ad5) carrying the mda-7 or Luc genes linked to an internal CMV-IE promoter and followed by SV40 polyadenylation (pA) signal have been constructed and will be referred to as Ad-mda7 and Ad-luc, respectively. Viruses were propagated in 293 cells and purified by chromatography.

C. Apoptotic Cell Staining

Sections were stained for apoptotic cell death using the terminal deoxynucleotide transferase (Tdt) (Boehringer Mannheim) kit and counterstained with methylene blue or methlene green as described (Fujiwara et al., 1994).

d. Western Blotting Analysis

Western blotting was performed as described above. Cells were harvested by trypsinization, washed with PBS and resuspended in 100 μl of lysis buffer (62.5 mM Tris-Hcl, 2% SDS, 10% glycerol, 4 M urea). Cell extracts were homogenized with sonicator for 30 sec and after an hour incubation on ice, cell extracts were spun for 5 min at 14000 rpm at 4° C. Cell extracts were collected and stored in −70° C. Protein concentrations of all extracts were determined using the Bio-Rad protein determination kit (Bio-Rad). Each of 50 μg protein samples were diluted into 20 μl with lysis buffer and 5% of 2-Mercapto Ethanol (Bio-Rad), and heated in a water bath at 95° C. for 5 min. Then protein extracts were separated on a 10% SDS-PAGE gel in a dual vertical slab gel electrophoresis cell (Bio-Rad).

Proteins were transferred from gel to nitrocellulose membrane (Hybond-ECL membranes). Proteins were blocked in a blocking solution (5% dry milk and 0.3% Tween 20 in PBS) for 1 hour at room temperature. Then membranes were incubated with primary antibody. Horse raddish peroxidase labeled secondary antibodies were applied and Enhanced chemiluminescence Western Blotting detection system (Amersham) was applied for 30 second and proteins were then visualizen on Amersham Hyperfilm enhanced chemiluminescence film using exposure time varying 30 sec to 30 min.

e. Evaluation of Tumor Growth and Treatments in Vivo

Prior to the start of all experiments involving subcutaneous tumor growth and treatments, nu/nu mice were irradiated (3.5 Gy) using a cesium source to enhance tumor uptake. In all the experiments, 5×10⁶ tumor cells (H1299, A549) suspended in 100 μl sterile phosphate buffered saline (PBS) were injected into the right dorsal flank. When the tumor had reached a size of 50-100 mm³, animals were randomized into three groups (n=8 animals/group) and treatment initiated as follows. Group 1 received no treatment, Group 2 received Ad-Luc (5×10⁹ vp/dose) and Group 3 received Ad-mda-7 (5×10⁹ vp/dose) every alternate day for a total of three doses. Intratumoral injections were performed under anesthesia using methoxyflurane (Schering Plough, Kenilworth, N.J.) as per institutional guidelines. Tumor measurements were recorded every other day without knowledge of the treatment groups, and the volume was calculated using the formula V (mm3)=a×b²/2, where “a” is the largest dimension and “b” is the perpendicular diameter. Antitumor efficacy data are presented as cumulative tumor volumes for all animals in each group to account for both size and number of tumors.

f. Immunohistochemical Analysis

Tumors established subcutaneously in nude mice were obtained and fixed in 10% buffered formalin, paraffin embedded and cut as 4 μm thick sections. Sections were stained for mda-7 gene expression. Briefly, tissue sections were treated with 0.3% H₂O₂ in methanol for 30 minutes to block endogenous peroxidase activity and were subsequently incubated with normal goat serum for 30 minutes at room temperature. Following incubation, slides were treated with rabbit polyclonal anti-MDA-7 antibody (1:5000 dilution) for 60 minutes. After 30 minutes incubation with anti-rabbit secondary antibody (provided with ABC kit, Vector) protein expression of MDA-7 in tissues were detected with DAB by enhancement with avidin-biotin reaction ABC kit. The slides will be counterstained with hematoxylin and then mounted with Aqua-mount (Lerner Labs., Pittsburgh, Pa.). The number of tumor cells staining positive for MDA-7 were analyzed under bright field microscopy and quantitated in a blind fashion using image analysis and statpro software. A total of at least five fields per specimen were analyzed.

g. TUNEL Staining

Tissue sections obtained from subcutaneous tumors were stained for apoptotic cell death using the terminal deoxynucleotide transferase kit (Tdt) (Boehringer Mannheim). In all the staining procedures, appropriate negative controls were included. The number of tumor cells staining TUNEL positive were analyzed under bright field microscopy and quantitated in a blind fashion using image analysis and statpro software. A total of at least five fields per specimen were analyzed.

h. Statistical Analysis

The statistical significance of the experimental results was calculated using Student's t-test for tumor measurements.

2. In vivo Suppression of Local Tumor Growth by Ad-mda7

The therapeutic effect of the mda-7 gene on H1299 and A549 subcutaneous tumors was evaluated in nude mice. Mice bearing each tumor cell type (H1299 and A549) were divided into three groups, one receiving no treatment, one treatment with Ad-Luc, and one treatment with the Ad-mda-7 daily for a total of three doses (5×10⁹ viral particles/dose). A significant growth inhibition of H1299 tumors and A549 tumors was observed in mice treated with the Ad-mda-7 compared with the tumor growth in the two control groups for each tumor type.

Further evidence that the observed therapeutic effect was due to mda-7 gene expression was obtained by removing subcutaneous tumors 48 hours after injection and analyzing them by immunohistochemistry. mda-7 gene expression was observed in tumor cells in animals receiving the Ad-mda7, as compared to no positive staining in control tumors that were either not treated or treated with Ad-Luc.

MDA-7 gene expression in situ results in apoptotic cell death through caspase-3 and Apo2/TRAIL activation. To understand the mechanism of tumor inhibition mediated by mda-7, subcutaneous tumors harvested at 48 hours following the last treatment were analyzed for apoptotic tumor cell death by TUNEL staining. Tumors from control mice that were either untreated or treated with Ad-Luc showed minimal apoptotic cell death while tumors from animals treated with Ad-mda-7 demonstrated extensive apoptosis.

Since apoptosis is mediated by activation of caspases, tumor tissues were examined for caspase-3, a downstream caspase. Activated form of caspase-3 was observed in tissues treated with Ad-mda-7 while no caspase-3 activation was observed in the tissues from control mice. Similarly, activation of Apo2/TRAIL was observed in tumors expressing mda-7. In contrast, TRAIL expression was not observed in tumors that were not treated or treated with Ad-luc.

3. MDA-7 Expression Results in Upregulation of Costimulatory Molecules

The ability of dying tumor cells in situ to activate costimulatory molecules, B7 and ICAM, was investigated. Subcutaneous tumors injected with Ad-MDA7 or Ad-Luc were harvested 48 hrs following the last dose and analyzed by immunohistochemistry. Expression of B7 (7.1 and 7.2) and ICAM was observed in tumors expressing MDA-7 while no expression was observed in tumors treated with Ad-Luc.

4. Expression of MDA-7 in in situ Tumor Inhibits Angiogenesis

To further determine the tumor suppressive effects of mda-7, subcutaneous tumors were analyzed for CD31 expression, a marker frequently used to identify angiogenesis in tumors. Subcutaneous tumors treated with Ad-mda-7 demonstrated fewer numbers of blood vessels when compared to tumors treated with Ad-luc or no treatment groups.

### EXAMPLE 7

### Efficacy of Ad-mda7 to Prevent Metastatic Spread of Tumor

Experiments have demonstrated that Ad-mda7 can inhibit metastatic spread of lung cancer tumors in vitro. Further experiments will be performed using melanoma cell lines to evaluate the ability of MDA-7 to prevent the metastatic spread of melanoma tumors. Techniques and protocols discussed previously will be employed.

Human melanoma xenografts will be established by subcutaneous injection of human melanoma cells (1×10⁶ cells) into the flanks of nude mice. TXM-1 or TXM-18 cells may be used. Once the tumor reaches 5 mm mean diameter, increasing doses of Ad-mda7 or control Ad-luc will be injected into the tumors. Doses of 3×10⁷ to 3×10⁹ pfu will be tried. Adenoviral vector will be delivered in three injections of approximately 33 ml, total 100 ml, intralesionally. Each injection will be orthogonally oriented to the preceding injection to ensure efficient tumor coverage. After establishment of the appropriate dose, tumor xenografts will be treated with a single 100 ml dose or multiple fractional doses equaling 100 ml over a three day time period to assess the effectiveness of the described administration regimens. Following these studies, a comparison between single dose administration versus multiple dose administration will be performed, with a dose being defined as 100 ml injection of the previously optimized concentrations in pfus. Efficacy studies will consist of the treatment of tumor xenografts following the established adenoviral concentrations and treatment regimen for 3 to 5 days. Efficacy will be assessed by the reduction in tumor size. Tumor size will be determined by the direct measurement of tumor diameters.

Ad-mda7 treated tumors will be evaluated for expression of MDA-7 protein and apoptosis induction. Immunohistochemical detection of MDA-7 and TUNEL assay detection of apoptosis will be used to evaluate the efficacy of Ad-mda7 treatment at the cellular level. An MDA-7 antibody that specifically recognizes MDA-7 protein will be employed for immunohistochemistry procedures. Endothelial cells in the melanoma xenografts will be detected with antibodies directed against mouse CD-31. Areas of the tumor sections with high numbers of capiallaries and small venules will be found by scanning the sections at low power (×40 and ×100). In these areas individual vessels will be counted in ×200 magnification fields, and average scores recorded for the treated and untreated tumor samples. This method has been used to compare blood distribution and density in human xenografts in nude mice (Yoneda et al., 1998).

### Example 8

### Modulation of Growth Factors During Ectopic Expression of Mda-7

Because it has been hypothesized that MDA-7 has an autocrine/paracrine activity, the effect of Ad-mda7 on melanoma cells will be evaluated with respect to the secretion of factors involved in the progression of melanoma. ELISA assays will be used to address the release of these soluble mediators, such as different types of TGF-β1, IL-8, IL-10, and bFGF. Melanoma cells lines and normal cells will be treated with Ad-mda7, Ad-luc, or diluent control and then monitored for modulation of growth factor levels in culture supernatant after 24-48 hours. Immunoblotting on the lysates may also be performed at various times post-treatment.

### Example 9

### Ad-mda7 Enhances Activity of Herceptin

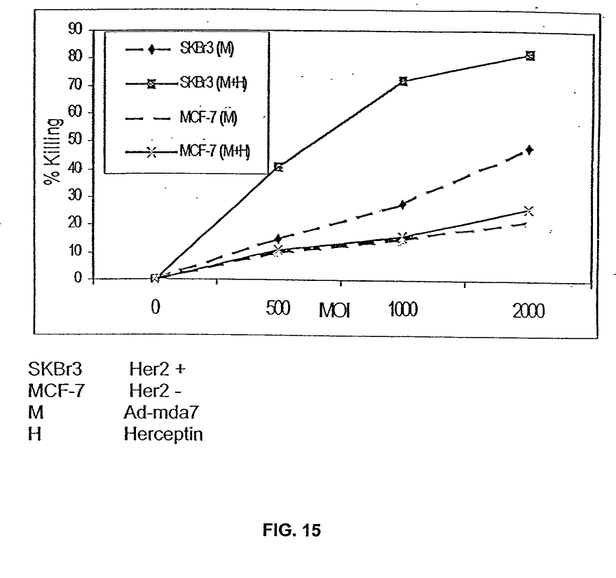

The breast cancer SkBr3 (Her2+) and MCF-7 (Her2-) cell lines were both obtained from ATCC. Cells were plated at a density of 1000 cells/well in Nunc 2-chamber slides and propagated in DMEM medium with 10% FBS. The following day, the cells were left untreated or treated with Ad-mda7 at (increasing MOIs: 0, 500, 1000 and 2000 vp/cell) without (M series) or with Herceptin (M+H series) at a final concentration of 1 μg/mL. The cells were washed after 3 hours and growth media (with or without Herceptin, as indicated) was replaced. Three days later viable cells were counted using the trypan blue exclusion assay (average of 3-4 fields) and plotted as shown in FIG. 15. Herceptin alone yields approximately 12% dead cells in both cell lines. However, Ad-mda7 appeared to enhance the killing effect of Herceptin in breast cancer cell lines.

### Example 10

### Ad-mda7 Inhibits Human Lung Cancer Growth and Angiogenesis

1. Materials and Methods

Cell culture. Human NSCLC cell line A549 (adenocarcinoma) was obtained from the American Type Culture Collection (Rockville, Md.). The human large cell lung carcinoma cell line (H1299) was a gift from Dr. A. Gazdar and Dr. J. D. Minna (University of Texas Southwestern Medical Center, Dallas, Tex.). Normal human bronchial epithelial cells (NHBE), and human umbilical vein endothelial cells (HUVEC) were purchased from Clonetics (Walkersville, Md.). Tumor cells were maintained in RPMI1640 medium containing 10% fetal bovine serum (GIBCO-BRL, Grand Island, N.Y.), antibiotics (GIBCO), and L-glutamine, and normal cells were grown under the conditions recommended by Clonetics. Before starting the experiments, the cells were verified to be free of mycoplasma; cells were used in the log phase of growth. Construction of the recombinant adenoviral vector. Viruses were propagated in human embryonic kidney 293 cells and purified by chromatography.

Cell Proliferation Assay. Tumor cells (H1299 and A549) and normal cells (NHBE) were seeded at 5×10³ cells/well in ninety six-well tissue culture plates. The following day, cells were infected with Ad-mda7 or Ad-luc at MOI of 5000 viral particles (vp)/cell. Following transfection, cells were replenished with complete medium. Seventy-two hours after infection, cell viability was determined by MTT assay as recommended by the manufacturer (Boehringer Mannheim, Indianapolis, Ind.).

Apoptotic Cell Staining (Hoechst staining). Cells were seeded in two-well chamber slides at a density of 1×10⁵ cells/well and infected with Ad-mda7 or Ad-luc (5000 vp/cell). Seventy-two hours after infection, cells were incubated with Hoechst No. 33342 (Sigma, St. Louis, Mo.) for 15 minutes, washed with phosphate-buffered saline (PBS) twice, and observed under a fluorescent microscope.

Tube Formation Assay. Human umbilical vein endothelial cells (HUVECs; Clonetics) were seeded on 1% gelatin-coated plates and incubated at 37° C. for 24 hours. After incubation, cells were infected for 1 hour with Ad-luc or Ad-mda 7 at 10,000 vp/cell in serum-free medium. Cells exposed to medium alone served as negative controls while cells exposed to Suramin (50 μM) served as positive controls. After a 48-hour incubation period (37° C. in serum-containing medium), infected cells were harvested, counted, and added to Matrigel-coated 24-well plates in triplicate (1.2×10⁵ cells per well). Twenty-four hours later, cells were fixed with 10% buffered formalin and examined for differentiation (tube formation) by using an Olympus IX-70 inverted bright-field microscope at 4× and 10× magnification.

Evaluation of Tumor Growth in vivo. Before the start of all experiments involving subcutaneous tumor growth and treatments, nu/nu mice were irradiated (3.5 Gy) from a cesium source to enhance tumor uptake. In all the experiments, 5×10⁶ tumor cells (A549, H1299) suspended in 100 μl sterile PBS were injected subcutaneously into the right dorsal flank. When the tumor had reached a size of 50-100 mm³, animals were randomized into three groups (n=8 animals/group) and treatment was initiated as follows. Group 1 received no treatment; Group 2 received Ad-luc (5×10⁹ vp/dose); and Group 3 received Ad-mda 7 (5×10⁹ vp/dose); all treatments were given every other day for a total of three doses. Intratumoral injections were performed under methoxyflurane anesthesia (Schering Plough, Kenilworth, N.J.) per institutional guidelines. Tumor measurements were recorded every other day without knowledge of the treatment groups, and tumor volumes were calculated by using the formula V (mm₃)=a×b²/2, where “a” is the largest dimension and “b” is the perpendicular diameter (Georges et al., 1993). Antitumor efficacy data are presented as average tumor volumes for all animals in each group to account for both size and number of tumors.

Immunohistochemical Analysis. Xenograft tumors established in nude mice were harvested and fixed in 10% buffered formalin, embedded in paraffin, and cut in 4-μm sections. Briefly, tissue sections were treated with 0.3% H₂O₂ in methanol for 30 minutes to block endogenous peroxidase activity, then incubated with normal goat serum for 30 minutes at room temperature. Following incubation, slides were treated with rabbit polyclonal anti-MDA7 antibody (Introgen Therapeutics, Houston, Tex.) for 60 minutes. After 30 minutes incubation with anti-rabbit secondary antibody (provided with ABC kit, Vector Labs, Burlinghame, Calif.), expression of the MDA-7 protein in tissues were detected with DAB by enhancement with avidin-biotin reaction ABC kit. The slides were then counterstained with hematoxylin and mounted with Aqua-mount (Lerner Labs., Pittsburgh, Pa.). Similar staining procedures were followed using anti-mouse CD31 (1:500, Pharmingen, San Diego, Calif.) and anti-human TRAIL (1:1000, Pharmingen, San Diego, Calif.) antibodies. Negative controls included tissue sections stained without primary antibody. Tissue sections were analyzed, quantiated and results interpreted in a blind fashion.

TUNEL staining. Tissue sections obtained from subcutaneous tumors were stained to detect apoptotic cell death using the terminal deoxynucleotide transferase (Tdt) kit (Boehringer Mannheim, Indianapolis, Ind.) as described previously (Fujiwara et al., 1993 and 1994). In all the staining procedures, appropriate negative controls were included.

Statistical Analysis. Student's t-test was used to calculate the statistical significance of the experimental tumor measurements.

2. Results

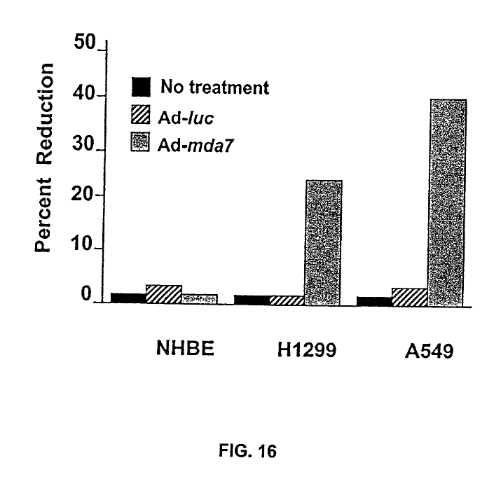

In vitro expression of MDA-7 inhibited tumor cell proliferation through apoptosis. To determine whether MDA-7 overexpression results in apoptosis, lung tumor cells (Hi299 and A549) and lung bronchial epithelial cells (NHBE) infected with Ad-mda7 or Ad-luc (5000 vp/cell) were stained 72 hours after infection with Hoechst 33342 and observed under fluorescence microscopy. Tumor cells infected with Ad-mda7 demonstrated morphological changes consistent with apoptosis. Few of the tumor cells infected with Ad-luc demonstrated apoptotic changes. No apoptotic changes were observed in NHBE cells infected with Ad-mda7 or Ad-luc. Furthermore, analysis of the effects of MDA-7 overexpression on cell proliferation demonstrated significant inhibition of tumor cells with 27% inhibition of H1299 cells and 40% inhibition of A549 cells at seventy-two hours after infection (FIG. 16B). In contrast, NHBE cells showed no growth inhibition.

MDA-7 overexpression inhibited endothelial cell differentiation in vitro. The ability of Ad-mda7 to inhibit endothelial cell differentiation was evaluated in HUVEC cells. Ad-mda7 inhibits endothelial cell differentiation into capillary-like structures (tube-formation). Human umbilical vein endothelial cells (HUVEC) were treated with Suramin, Ad-luc (10,000 vp/cell) or Ad-mda7 (10,000 vp/cell), or not treated. Forty-eight hours later, cells were harvested, mixed with Matrigel, and observed for tube formation. Overexpression of MDA-7 resulted in inhibition of endothelial tube formation, an effect similar to that of Suramin, a known inhibitor of tube formation. In contrast, cells infected with control vector (Ad-luc) demonstrated no inhibition of tube formation. That the observed inhibition of tube formation by endothelial cells was due to MDA-7 overexpression and not due to cytotoxicity was determined by trypan blue exclusion assay for cell viability. More than 80% of cells expressing MDA-7 were viable. The inhibition of tube formation suggests that Ad-mda7 may possess antiangiogenic activity in vivo (see below).

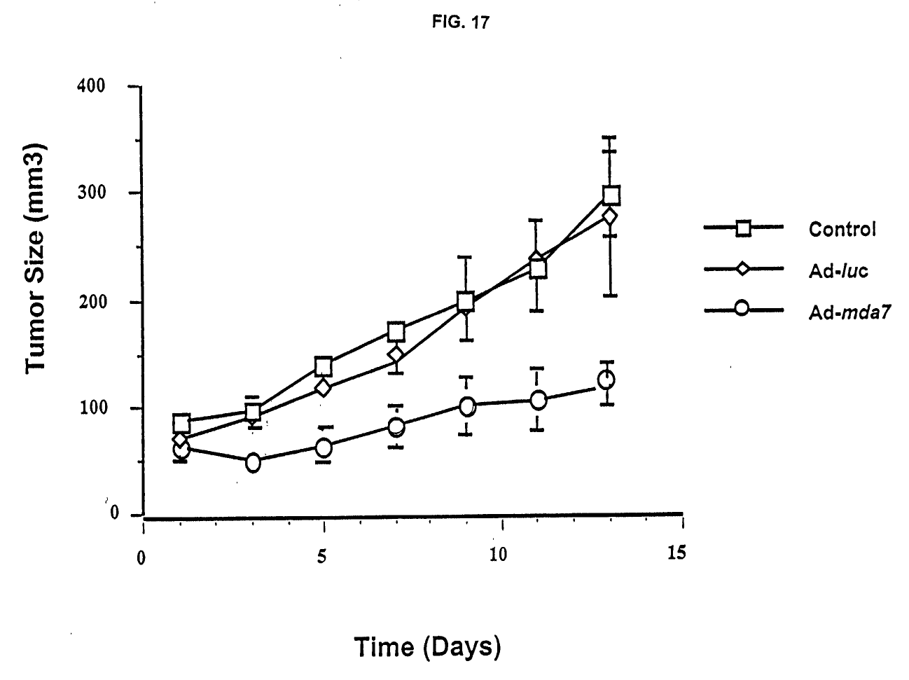

In vivo evaluation of local tumor growth suppression by mda-7. We assessed the therapeutic effects of intratumoral injection of Admda-7 on A549 and H1299 subcutaneous tumors in nude mice. Mice bearing experimentally induced xenograft tumors (A549 or H1299) were divided into three groups, one receiving no treatment, one treatment with Ad-luc, and one treatment with the Ad-mda7 daily for a total of three doses (5×10⁹ vp/dose). Significant inhibition of the growth of both H1299 tumors (p=0.01) and A549 tumors (p=0.001) was observed in mice treated with Ad-mda7 but not in the control groups for either tumor type (FIG. 17).

Further evidence that this therapeutic effect was due to MDA-7 overexpression was obtained by removing subcutaneous tumors 48 hours after treatment and subjecting them to immunohistochemical analysis. Strong MDA-7 expression (15%) was observed in tumor cells from animals that received Ad-mda7. MDA-7 expression was not detected in tumors that were either not treated or treated with Ad-luc (FIG. 17). MDA-7 expression was primarily observed in the cytoplasm with very little expression in the nucleus. In addition, extracellular staining was observed in some areas.

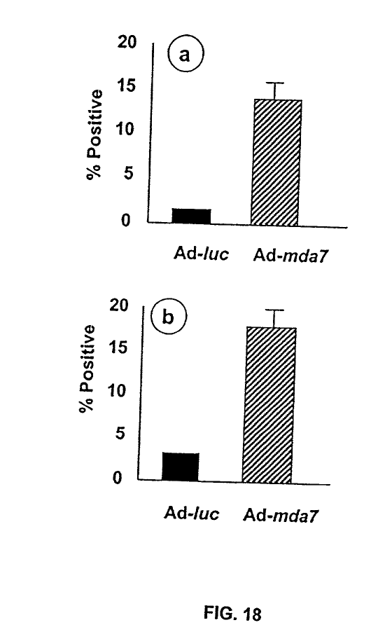

To understand the mechanism of tumor inhibition mediated by mda-7, subcutaneous tumors harvested 48 hours after the last treatment were analyzed for apoptotic tumor cell death by TUNEL staining. Tumors from mice treated with Ad-luc showed minimal apoptotic cell death (3%), whereas tumors from animals treated with Ad-mda7 demonstrated extensive apoptosis (17%) (FIG. 18).

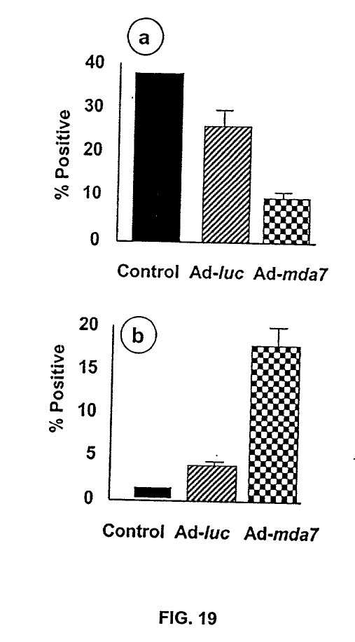

Overexpression of MDA-7 induced downregulation of CD31 and upregulation of TRAIL in experimental tumors. To further define the tumor-suppressive effects of mda-7, subcutaneous tumors from H1299 were analyzed for expression of CD31, a marker routinely used to identify neoangiogenesis in tumors, and TRAIL. Tumors treated with Ad-mda7 had significantly lower levels (9%) of CD31 indicating fewer blood vessels than in tumors treated with Ad-Luc (28%) or no treatment (39%). Similarly, analysis for expression of TRAIL, a promoter of apoptosis, demonstrated higher levels of TRAIL expression in tumors treated with Ad-mda7 (20%) than in tumors that were treated with Ad-luc (4%) or untreated (1%) (FIG. 19).

### Example 11

### Mda-7 is Effective in Human Patients

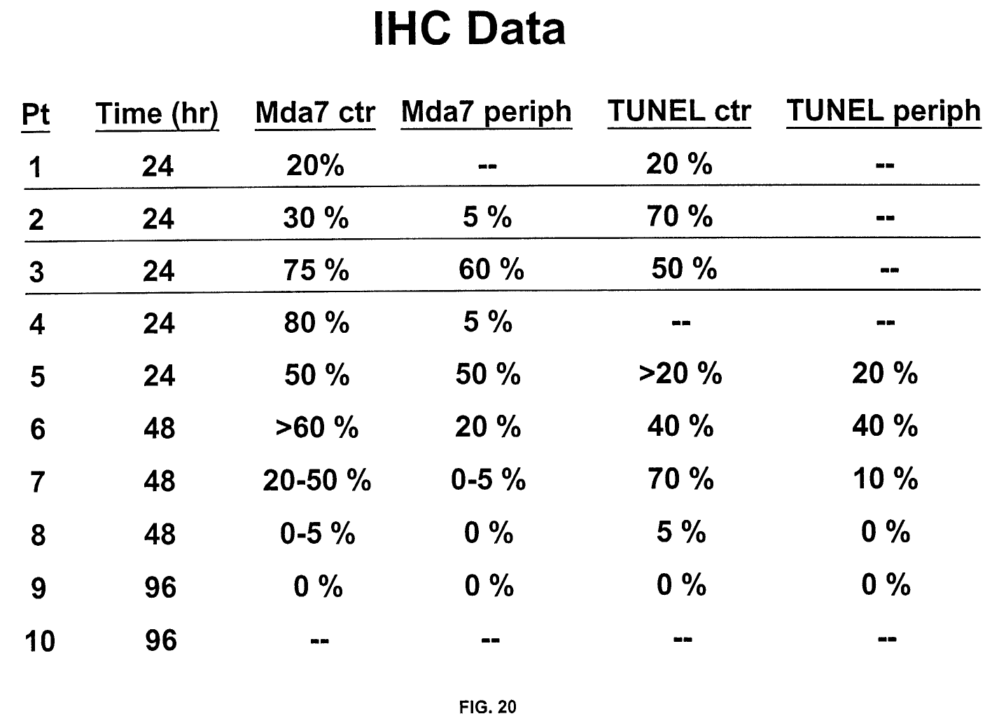

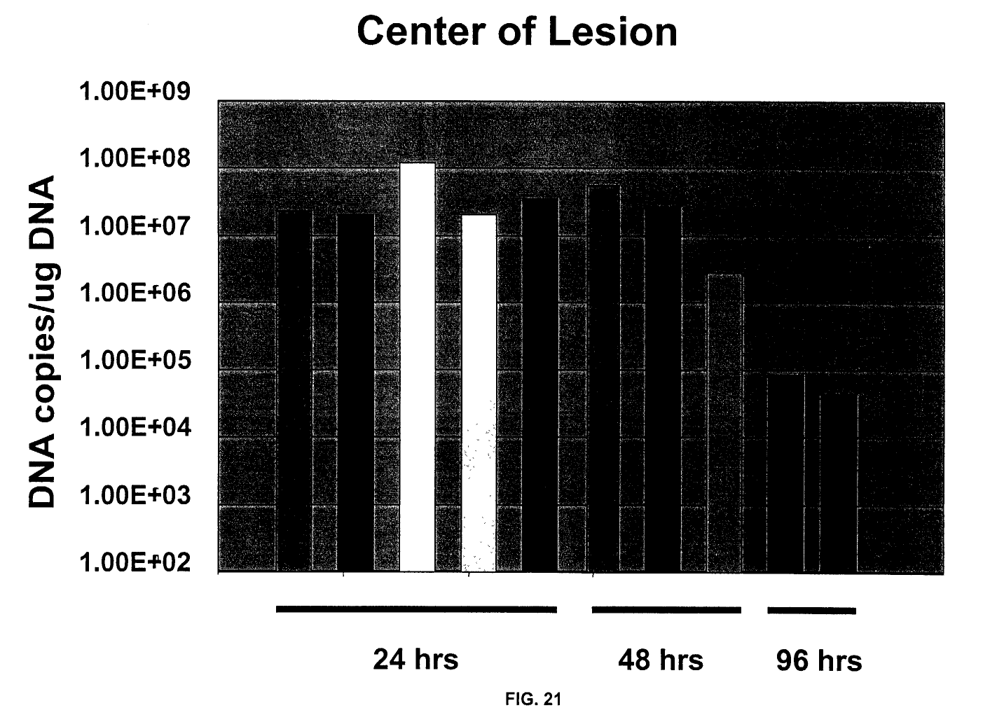

Ten patients with various tumors (including head and neck, breast, melanoma, colon, renal, non-Hodgkin's lymphoma, hepatoma) were treated with a single injection of Ad-mda7 (construct as described above). Tumors were resected 24-96 hours later and analyzed for MDA-7 expression by immunohistochemistry and for apoptosis by TUNEL assays. Tumors were sections so the center and periphery of the lesions could be evaluated. Pre-treatment or non-injected tumors were negative for MDA-7 immunohistochemistry. After Ad-mda-7 injection, high levels of transgenic MDA-7 protein were detected and high levels of apoptosis were observed (FIG. 20). In some patients, MDA-7 protein and TUNEL positivity was detected at the periphery of the tumor (>1 cm from injection). Furthermore, PCR amplification for mda-7 sequences showed high levels of Ad-mda7 DNA in the center of injected lesions (FIG. 21).

### Example 12

### Mda-7 Protein May Be Administered as Treatment

HUVEC cells were administered increasing amounts of MDA-7 protein purified from 293-mda7 cells. Doses that were evaluated ranged from 0.5-100 ng/ml. The ED₅₀ of MDA-7 ranged from 5-50 ng/ml. Endothelial differentiation in the cells was inhibited by MDA-7 protein, but not control cells, based on tube formation.

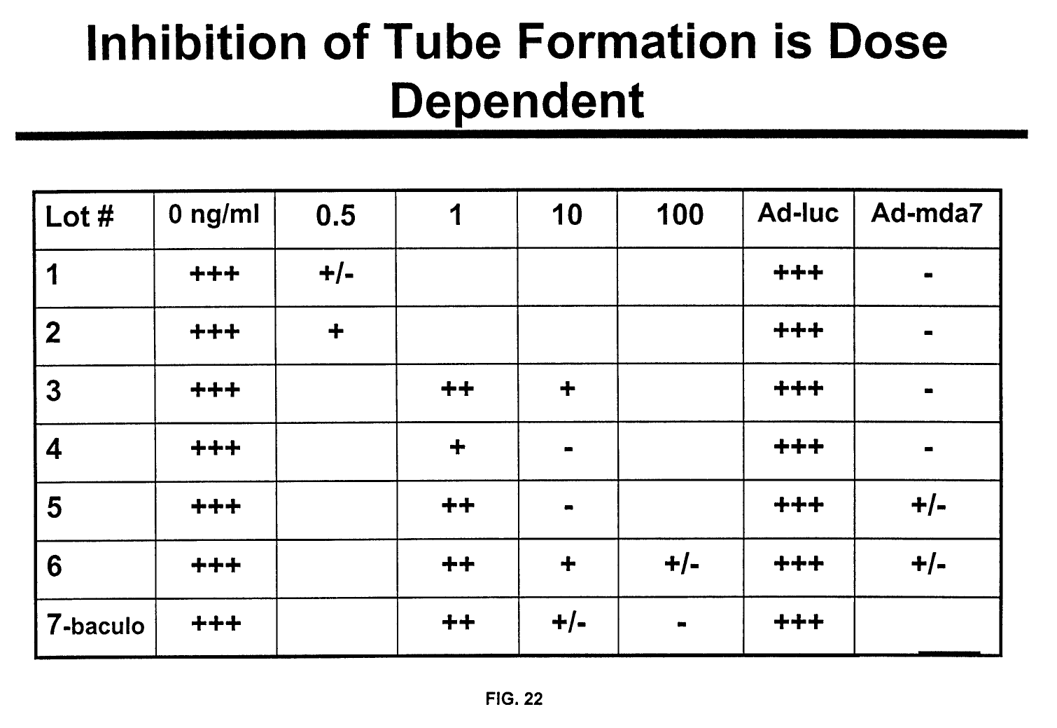

HUVEC cells were given varying doses of MDA-7 protein purified from 293-mda7 cells (lots 1-6) or from baculovirus exoressing mda-7. A positive control of Ad-mda7 and a negative control of Ad-luc were included in most assays. Ad-mda7 inhibited tube formation and MDA-7 protein also inhibited tube formation at doses as low as 0.5-10 ng/ml (FIG. 22).

### Example 13

### Mda-7 has Cytokine Activity

1. Materials and Methods

Activation of PBMC: PBMC were isolated from the peripheral blood of normal healthy donors by centrifugation over Histopaque (Sigma, St. Louis, Mo.). Cells were cultured at a concentration of 1×10⁶ cells/ml in RPMI-1640 based media supplemented with L-glutamine, Hepes, penicillin, streptomycin, and 10% human AB serum (Pelfreez, Brown Deer, Wis.) for 72 hr in the presence of PHA-P at 5 μg/ml or LPS 10 μg/ml (both from Sigma, St. Louis, Mo.). Four hours prior to harvest Brefeldin A (BFA, Sigma-Aldrich) was added at a final concentration of 10 μg/ml. The supernatants as well as cells were then harvested.

For PBMC subclass studies, the PHA stimulated cells were separated into CD3+, CD 19+, and CD56+enriched populations by one round of positive selection by magnetic cell sorting using a MiniMax (Miltenyi Biotec, Inc., Sunnyvale, Calif.). Peripheral blood monocytes were isolated by adherence to chamber slides (Nalge Nunc International, Naperville, Ill.). Total PBMC were incubated in these chambers at a concentration of 1×10⁶ cells/ml with or without LPS for 72 h. The purity of these populations was determined by staining with FITC- or PE-conjugated monoclonal antibodies against CD3, CD19, CD56, and CD14 (BD Immunocytometry, Mountain View, Calif.). The cells were analyzed cytofluorometrically using a FACsan with Cell Quest software (BD Immunocytometry). CD3 enriched subpopulation contained 97% CD3+, the CD19 subpopulation contained 71% CD19+ cells, and the CD56 enriched population contained 91% CD56+ cells (the contaminants are CD3+). Human recombinant IL-10 was purchased from R&D Systems (Minneapolis, Minn.), and IL-2 was a generous gift from Cetus/Chiron corporation (Emeryville, Calif.).

Immunohistochemical Staining for MDA-7. Immunostaining of human PBMC or subclasses was performed using mouse monoclonal antibody against human MDA7 (Introgen Therapeutics Inc., Houston, Tex.), employing the avidin-biotinylated-peroxidase complex method by a method optimized by us previously for melanocytes and melanoma cells (Ekmekcioglu, 2001).

ELISA assays. The ELISA reaction to detect human MDA-7 was carried out in 96-well plates using standard techniques and an antibody pair selected for sentivity. Elisa assays for cytokines were perfomed in an identical manner, employing commercially available kits as designated in the figure legends.

Western blotting. The activated PBMC were washed once in PBS, resuspended in modified RIPA buffer (TBS, pH 7.6, 1% NP-40, 0.5% sodium deoxycholate, 0.1% SDS, 50 mM sodium fluoride, 0.2 mM aprotinin, 1 mM leupeptin) and rocked at 4° C. for 20 minutes. Lysates were cleared by a 30 min centrifugation at 16,000×g at 4° C. Protein concentrations were determined with the DC Protein Assay (Bio-Rad, Hercules, Calif.) and samples were boiled for 5 minutes in an equal volume of sample buffer (62.5 mM Tris-HCl, pH 6.8, 20% glycerol, 2% SDS, 5% β-mercaptoethanol). Samples were separated by SDS-PAGE on a 12% gel and transferred to nitrocellulose. The membrane was blocked for 30 minutes with blocking buffer and incubated in a rabbit polyclonal MDA-7 Ab (Introgen Therapeutics, Houston, Tex.) in blocking buffer. Subsequently the membranes were washed twice in PBST, incubated at 1:2000 with HRP conjugated goat anti-rabbit secondary Ab. Blots were developed with ECL reagent (Amersham Pharmacia Biotech, Piscataway, N.J.). Membranes were incubated in stripping buffer (62.5 mM Tris-HCl, pH 6.7, 2% SDS and 100 mM β-mercaptoethanol) for 30 min at 60° C., washed three times with PBST for 10 minutes each, and reprobed with anti-actin antibody(1:1000).

Purification of human MDA-7. The full length cDNA of mda-7 was cloned into the pCEP4 FLAG vector (Invitrogen), which uses the CMV promoter to drive mda-7 gene expression. The plasmid was transfected into HEK 293 cells and antibiotic resistant stable subclones were isolated using hygromycin (0.4 ug/mL). Purification of MDA-7 protein was performed using the HEK 293 cell supernatants collected from viable cells in log phase growth. The crude supernatant was determined by ELISA to contain approximately 30 ng/ml MDA-7. No actin was found in the supernatant (data not shown), strongly supporting the premise that the MDA-7 material derived was not from dead cells, but secreted from completely viable and healthy cells. Supernatant containing the secreted MDA-7 was supplemented with protease inhibitors (1 μg/ml leupeptin, 1 μg/ml pepstatin, and 0.5 mM PMSF) and 0.05% sodium azide and were concentrated 10-fold with an Amicon stirred cell on a YM10 membrane. Ten ml aliquots of concentrated supernatants were separated over an S200 Superdex prep grade column in PBS pH 7.4 and fractions identified to contain MDA-7 by Western and ELISA were pooled. After buffer exchange on an Amicon stirred cell to 50 mM MES, pH 6, a second purification step was performed using a BioRad S column. Column conditions consisted of a 0-90 mM NaCl gradient, a 5 min hold at 90 mM NaCl, a 30 min gradient 90 mM-250 mM gradient at 1 ml/min and a 5 min hold at 250 mM NaCl. The entire purification was carried out at 4° C. and MDA-7 identified using ELISA and Western blotting procedures. The final samples were at least 300 ng/ml of MDA-7 as determined by ELISA, and the specific activity was enriched at least 28-fold over the starting supernatant material based on the elimination of extraneous protein.

2. Results

Human PBMC can be induced to express MDA-7 protein. Fresh normal donor human PBMC were either unactivated or treated with the polyclonal stimuli of PHA or LPS, and were examined for intracellular MDA-7 protein expression by immunoblotting and by immunohistochemistry. Untreated PBMC generally do not express detectable levels of MDA-7 protein. However, after treatment with PHA or LPS for 72 hours, a variable but detectable amount of MDA-7 protein was evident from multiple donors (3 of 5). In one of these experiments, a weaker band of MDA-7 was detectable in cultured unactivated PBMC, suggesting that in some individuals under certain circumstances, MDA-7 is expressed de novo, or that our blotting procedure may reflect a barely detectable baseline that is at the threshold of sensitivity of the immunoblotting methodology, and expression is upregulated by the stimulation. Specificity of the antiMDA-7 antibody was confirmed by total blocking with recombinant MDA-7 protein as previously published (Ekmekcioglu et al., 2001).

MDA protein is expressed by nonCD3 subsets. To determine whether MDA-7 is expressed by all stimulated PBMC at low levels, or whether certain subclasses were strongly positive, subclass analysis was performed using subsets selected from PHA stimulated PBMC. Positively selected monocytes (3/3 experiments) and CD3+ T cells (6/6 experiments) were routinely negative, but CD56+ and CD19+ subpopulations resulting from the same starting PBMC and separation procedures were unequivocally positive. Membrane staining was most evident in the CD56+ cells, and a granular location in both types of cells was observed.

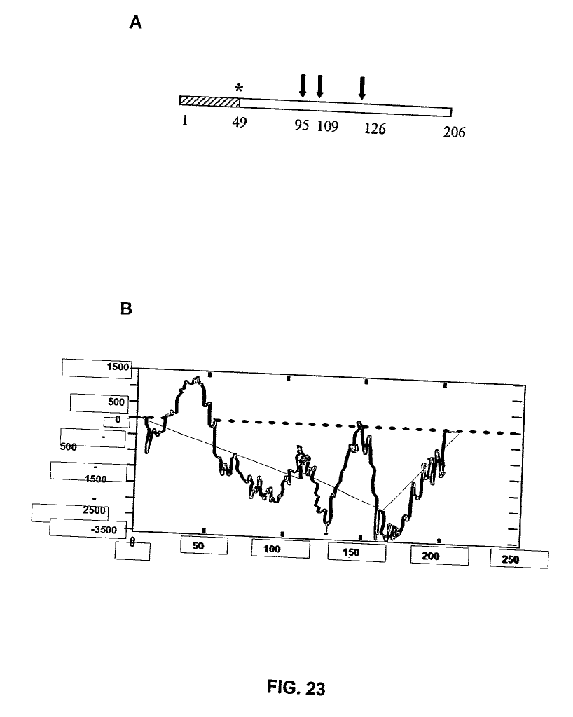

MDA-7 can be a secreted protein. One of the characteristics of a cytokine is its ability to be secreted. Usually, a short stretch of hydrophobic amino acids at the amino terminus of a protein signals and targets it to a secretory pathway. As depicted in FIG. 22A, the mda-7 cDNA sequence contains a leader sequence consisting of 49 amino acids; this is depicted in more detail in the hydrophobicity plot (FIG. 23B). The predicted cleavage site was determined by the von Heijne SignalP predictions program (Nielsen et al., 1997), however, to the best of our available information, this cleavage site in MDA-7 has not been confirmed experimentally. In order to demonstrate secretion of MDA-7 from mammalian cells, stable transfectants of 293 cells containing the human MDA-7 full-length cDNA were generated (Mhashilkar et al., 2001). Supernatants were analyzed for MDA-7 expression by the Western blot and four bands of MDA-7 protein were detected in the culture supernatants of MDA-7 transfected but not untransfected 293 cells. At this time, the molecular nature of the multiple size bands of MDA-7 after secretion is not clear. Based on the amino acid sequence, MDA-7 is expected to have a molecular weight of 18,419 and when containing the leader sequence it is 23,824 kDa (ProtParam tool). As with IL-10, homodimerization of MDA-7 is likely to occur; also the possibility of variable glycosylation for MDA-7 is also a consideration, and both of these postranslational modifications are being investigated. Many cytokines have been demonstrated to be glycosylated to varying degrees (May et al., 1991; Gross et al., 1989).

MDA-7 protein induces secondary cytokines, inhibited by IL-10. Another hallmark of the cytokine family is that of belonging in a cascade of additional molecules involved in cellular activation or inhibition. In order to address the biological function of MDA-7 as a cytokine, its induction of secondary cytokine secretion by PBMC was examined. Preliminary experiments using recombinant MDA-7 expressed in E. coli and S. cerevisiae showed that MDA-7 could induce robust production of IL-6, TNFα, and IFNγ, very low levels of GM-CSF and IL-10, and no IL-2, IL-4, and IL-5. However, very high doses (μg/ml quantities) of bacterial MDA-7 were required to stimulate a response, possibly due to improper folding or glycosylation of the recombinant protein. Therefore stably transfected human embryonic kidney cells, 293 cells, were prepared, and secreted and purified MDA-7 was used in these reported activation experiments.

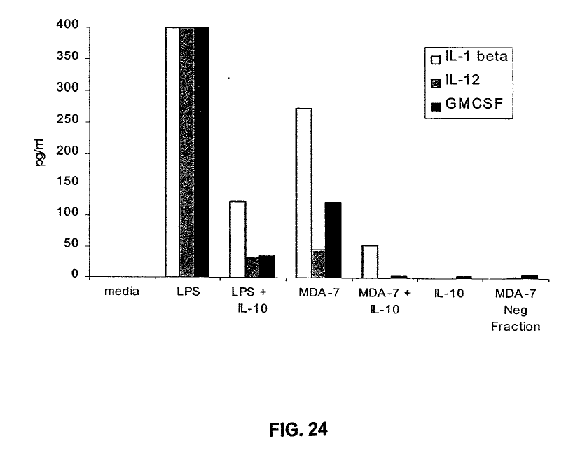

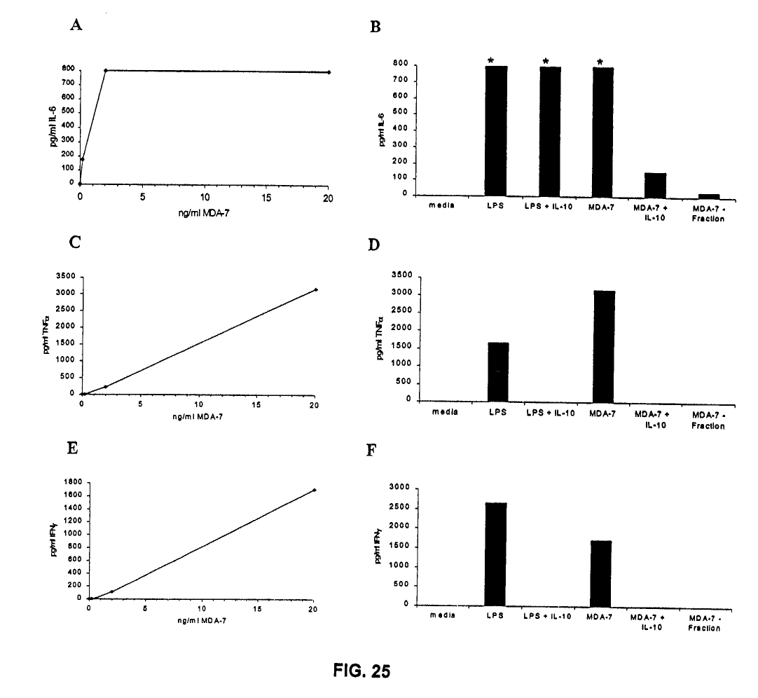

FIG. 24 shows MDA-7 induction of IL-6, TNFα, and IFNγ, with maximal IL-6 secretion stimulated by only 200 pg/ml of MDA-7. At 2 ng/ml of MDA-7, the IL-6 secretion is already greater than 800 pg/ml (above the standard curve of the ELISA kit). The higher dose of 2 ng/ml of MDA-7 are required to achieve optimal levels of TNFα, and IFNγ secretion, as shown in FIG. 25C, E. LPS, a known inducer of inflammatory cytokines, was used as a positive control, and in the induction of TNFα, MDA-7 was a more potent inducer than the positive stimulation control LPS. A similar pattern of MDA-7 stimulation of cytokine production was observed with IL-1β, IL-12 and GM-CSF as shown in FIG. 25. Values in a similar range of the amount of cytokines from the donor shown in FIG. 25 were detected in supernatants from three additional donors. Using either polyclonal antisera, or a monoclonal antibody specific for MDA-7, adsorption of the MDA-7 protein by >90% as determined by ELISA resulted in significant reduction to total loss of secondary induction of IL-6, and IFNγ (2/2 experiments), indicated that the induction of these secondary cytokines was due to the MDA-7 and not a possible contaminant.

Because MDA-7 is a member of the IL-10 family, and IL-10 is known to be a premier immunosuppressive cytokine, it was curious to us that MDA-7 stimulated the production of proinflammatory cytokines. Therefore, we hypothesized that IL-10 and MDA-7 may be antagonists. To test this hypothesis, human recombinant IL-10 was added to the PBMC cultures stimulated by MDA-7. It was found that under the conditions used, IL-10 completely abrogated TNFα and IFNγ induction by MDA-7 and partially blocked IL-6 induction by MDA-7 (FIG. 24). As a positive control, the IL-10 also prevented the production of two of these three cytokines in response to LPS. The lack of IL-10 inhibition of LPS induced IL-6 secretion is probably due to the IL-6 values greatly exceeding the standard curve of the assay. IL-10 also partially inhibited IL-1β and GM-CSF production and completely inhibited IL-12 production (FIG. 25). As with any study using freshly isolated human PBMC there was some variability from donor to donor, but the result of MDA-7 inducing secondary cytokines and inhibition by IL-10 was consistent in all donors tested and all experiments.

MDA-7 does not appear to function as a growth factor for human PBMC. Some cytokines can also function as growth factors. Therefore the proliferative simulation function of MDA-7 was addressed using PBMC. IL-10 was included as a negative cytokine control. PHA was used as positive control and induced a robust uptake of thymidine in all three donors. As expected, IL-10 did not induce increased thymidine uptake of PBMC over the course of four day. Our results show that MDA-7 did not induce significant proliferation during 4 days of coculture of the PBMC population in any of the three donors tested. Earlier studies employing recombinant MDA-7 (up to 5 μg/ml) expressed in E. coli or S. cerevisiae also did not show a proliferative response in human PBMC from three donors.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. 4,797,368
U.S. Pat. 4,870,287
U.S. Pat. 5,139,941
U.S. Pat. 5,399,363
U.S. Pat. 5,466,468
U.S. Pat. 5,543,158
U.S. Pat. 5,633,016
U.S. Pat. 5,641,515
U.S. Pat. 5,656,016
U.S. Pat. 5,697,899
U.S. Pat. 5,739,169
U.S. Pat. 5,760,395
U.S. Pat. 5,779,708
U.S. Pat. 5,770,219
U.S. Pat. 5,783,208
U.S. Pat. 5,797,898
U.S. Pat. 5,798,339
U.S. Pat. 5,801,005
U.S. Pat. 5,811,128
U.S. Pat. 5,824,311
U.S. Pat. 5,824,348
U.S. Pat. 5,830,880
U.S. Pat. 5,846,225
U.S. Pat. 5,846,233
U.S. Pat. 5,846,945
WO 0026368
WO 0005356
WO 9828425
WO 9807408
WO 9511986

Aksentijevich et al., Hum Gene Ther 7(9): 1111-22, 1996.

Angel et al., Cell 49(6): 729-39, 1987.

Angel et al., Mol Cell Biol 7(6): 2256-66, 1987.

Angiolillo et al., J Exp Med 182(1): 155-62, 1995.

Arap et al., Cancer Res 55(6): 1351-4, 1995.

Atchison et al., Cell 46(2): 253-62, 1986.

Austin-Ward et al., Rev Med Chil 126(7): 838-45, 1998.

Ausubel, ed., Current protocols in molecular biology, New York, John Wiley & Sons, 1996.

Baichwal, Vectors for gene transfer derived from animal DNA viruses: Transient and stable expression of transferred genes. Gene Transfer. R. Kucherlapate. New York, Plenum Press: 117-148, 1986.

Bakhshi et al., Cell 41(3): 899-906, 1985.

Banerji et al., Cell 27(2 Pt 1): 299-308, 1981.

Banerji et al., Cell 33(3): 729-40, 1983.

Berkhout et al., J Virol 63(12): 5501-4, 1989.

Blanar et al., Embo J 8(4): 1139-44, 1989.

Blumberg et al., Cell 104, 9, 2001.

Bodine et al., Embo J 6(10): 2997-3004, 1987.

Boshart et al., Cell 41(2): 521-30, 1985.

Bosze et al., Embo J 5(7): 1615-23, 1986.

Bourlais, et al., Prog Retin Eye Res 17(1): 33-58, 1998.

Braddock et al., Cell 58(2): 269-79, 1989.

Brizel et al., Semin Radiat Oncol 8(4 Suppl 1): 17-20, 1998.

Bukowski et al., Clin Cancer Res 4(10): 2337-47, 1998.

Bulla et al., J Virol 62(4): 1437-41, 1988.

Bulla, G. A. and A. Siddiqui (1989). “Negative regulation of the hepatitis B virus pre-S1 promoter by internal DNA sequences.” Virology 170(1): 251-60, 1989.

Caldas et al., Nat Genet 8(1): 27-32, 1994.

Caley et al., J Virol 71(4): 3031-8, 1997.

Campbell et al., Mol Cell Biol 8(5): 1993-2004, 1988.

Camper et al., Biotechnology 16: 81-7, 1991.

Campo et al., Nature 303(5912): 77-80, 1983.

Cao et al., J Exp Med 182(6): 2069-77, 1995.

Celander et al., J Virol 61(2): 269-75, 1987.

Celander, D., B. L. Hsu, et al., J Virol 62(4): 1314-22, 1988.

Chang et al., Mol Cell Biol 9(5): 2153-62, 1989.

Chang, Hepatology 14: 134A, 1991.

Chatterjee et al., Proc Natl Acad Sci U S A 86(23): 9114-8, 1989.

Chen et al., Cancer Res 55(19): 4230-3, 1995.

Chen et al., Mol Cell Biol 7(8): 2745-52, 1987.

Cheng et al., Cancer Res 54(21): 5547-51, 1994.

Chol et al., Eur J Biochem 239(3): 579-87, 1996.

Christodoulides et al., Microbiology 144(Pt 11): 3027-37, 1993.

Clapp et al., Endocrinology 133(3): 1292-9, 1993.

Clark et al., Hum Gene Ther 6(10): 1329-41, 1995.

Cleary et al., Cell 47(1): 19-28, 1986.

Cleary et al., Proc Natl Acad Sci U S A 82(21): 7439-43, 1985.

Coffin, Retroviridae and their replication. Fields Virology. Fields. New York, Raven Press: 1437-1500, 1990.

Cohen et al., J Cell Physiol Suppl Suppl 5: 75-81, 1987.

Costa et al., Mol Cell Biol 8(1): 81-90, 1988.

Couch, Am. Rev. Resp. Dis. 88: 394-403, 1963.

Coupar et al., Gene 68(1): 1-10, 1988.

Cripe et al., Embo J 6(12): 3745-53, 1987.

Culotta et al., Mol Cell Biol 9(3): 1376-80, 1989.

Culver et al., Science 256(5063): 1550-2, 1992.

Curiel, Gene transfer mediated by adenovirus-polylysine DNA complexes. Viruses in Human Gene Therapy. Vos. Durham, N.C., Carolina Academic Press: 179-212, 1994.

Curran, Semin Radiat Oncol 8(4 Suppl 1): 2-4, 1998.

Dandolo et al., J Virol 47(1): 55-64, 1983.

Davidson et al., J Immunother 21(5): 389-98, 1998.

Davis et al., J Virol 70(6): 3781-7, 1996.

De Waal Malefyt et al., J. Exp. Med. 174, 1209, 1991.

Deschamps et al., Science 230(4730): 1174-7, 1985.

Dumoutier et al., J of Immunol. 167, 3545, 2001.

Dumoutier et al., Proc. Natl. Acad. Sci. USA. 97, 10144, 2000.

Edbrooke et al., Mol Cell Biol 9(5): 1908-16, 1989.

Edlund et al., Science 230(4728): 912-6, 1985.

Ekmekcioglu et al., Intl. J. Cancer. 94, 54-59, 2001.

Ekmekcioglu et al., Melanoma Research 9, 261, 1999.

el-Kareh et a., Crit Rev Biomed Eng 25(6): 503-71, 1997.

Erlandsson, Cancer Genet Cytogenet 104(1): 1-18, 1998.

Fechheimer et al., Proc Natl Acad Sci U S A 84(23): 8463-7, 1987.

Felgner et al., Proc Natl Acad Sci U S A 84(21): 7413-7, 1987.

Feng et al., Nature 334(6178): 165-7, 1988.

Firak et al., Mol Cell Biol 6(11): 3667-76.

Flotte et al., Am J Respir Cell Mol Biol 7(3): 349-56, 1992.

Flotte et al., Proc Natl Acad Sci U S A 90(22): 10613-7, 1993.

Flotte, et al., Gene Ther 2(1): 29-37, 1995.

Foecking et al., Gene 45(1): 101-5, 1986.

Folkman et al., J Natl Cancer Inst 82(1): 4-6, 1990.

Fraley et al., Proc Natl Acad Sci U S A 76(7): 3348-52, 1979.

Friedmann, Science 244(4910): 1275-81, 1989.

Fujita et al., Cell 49(3): 357-67, 1987.

Fujiwara et al., Cancer Res 53(18): 4129-33, 1993.

Fujiwara et al., Cancer Res 54(9): 2287-91, 1994.

Fulci et al., Brain Pathol 8(4): 599-613, 1998.

Gabizon et al., Cancer Res 50(19): 6371-8, 1990.

Gallagher et al., Genes Immun. 1, 442, 2000.

Georges et al., Cancer Res 53(8): 1743-6, 1993.

Gertig et al., Semin Cancer Biol 8(4): 285-98, 1998.

Ghosh et al., Targeted Diagn Ther 4: 87-103, 1991.

Ghosh-Choudhury et al., Embo J 6(6): 1733-9, 1987.

Gillies et al., Cell 33(3): 717-28, 1983.

Gloss et al., Embo J 6(12): 3735-43, 1987.

Godbout et al., Mol Cell Biol 6(2): 477-87, 1986.

Gomez-Foix et al., J Biol Chem 267(35): 25129-34, 1992.

Good et al., Proc Natl Acad Sci U S A 87(17): 6624-8, 1990.

Goodbourn et al., Cell 45(4): 601-10, 1986.

Goodbourn et al., Proc Natl Acad Sci U S A 85(5): 1447-51, 1988.

Gopal et al., Mol Cell Biol 5(5): 1188-90, 1985.

Graham et al., Biotechnology 20: 363-90, 1992.

Graham et al., J Gen Virol 36(1): 59-74, 1977.

Graham et al., Manipulation of adenovirus vector. Methods in molecular biology: Gene transfer and expression protocol. Murray. Clifton, N.J., Humana Press. 7: 109-128, 1991.

Graham et al., Virology 52(2): 456-67, 1973.

Greene et al., Adv Exp Med Biol 254: 55-60, 1989.

Gross et al., FEBS Lett. 247, 323, 1989.

Grosschedl et al., Cell 41(3): 885-97, 1985.

Grunhaus et al., Seminars in Virology 3: 237-252, 1992.

Gupta et al., Proc Natl Acad Sci U S A 92(17): 7799-803, 1995.

Hanahan et al., Science 277(5322): 48-50, 1997.

Hanibuchi et al., Int J Cancer 78(4): 480-5, 1998.

Harland et al., J Cell Biol 101(3): 1094-9, 1985.

Hartmann et al., Br J Cancer 73(8): 896-901, 1996.

Hartmann et al., Int J Cancer 67(3): 313-7, 1996.

Haskell et al., Mol Reprod Dev 40(3): 386-90, 1995.

Haslinger et al., Proc Natl Acad Sci U S A 82(24): 8572-6, 1985.

Hauber et al., J Virol 62(3): 673-9, 1988.

Hellstrand et al., Acta Oncol 37(4): 347-53, 1998.

Hen et al., Nature 321(6067): 249-51, 1986.

Hensel et al., Lymphokine Res 8(3): 347-51, 1989.

Hermonat et al., Proc Natl Acad Sci U S A 81(20): 6466-70, 1984.

Herr et al., Cell 45(3): 461-70, 1986.

Herz et al., Proc Natl Acad Sci U S A 90(7): 2812-6, 1993.

Hesdorffer et al., DNA Cell Biol 9(10): 717-23, 1990.

Hirochika et al., J Virol 61(8): 2599-606, 1987.

Hirsch et al., Mol Cell Biol 10(5): 1959-68, 1990.

Ho et al., Cancer 83(9): 1894-907, 1998.

Holbrook et al., Virology 159(1): 178-82, 1987.

Hollstein et al., Science 253(5015): 49-53, 1991.

Holmgren et al., Cell 79(2): 315-28, 1994.

Hori et al., Cancer Res 51(22): 6180-4, 1991.

Horlick et al., Mol Cell Biol 9(6): 2396-413, 1989.

Horwich et al., J Virol 64(2): 642-50, 1990.

Howard et al., J. Clin. Immunol. 12, 239, 1992.

Huang et al., Cell 27(2 Pt 1): 245-55, 1981.

Hug et al., Mol Cell Biol 8(8): 3065-79, 1988.

Hui et al., Infect Immun 66(11): 5329-36, 1998.

Hussussian et al., Nat Genet 8(1): 15-21, 1994.

Hwang et al., Mol Cell Biol 10(2): 585-92, 1990.

Imagawa et al., Cell 51(2): 251-60, 1987.

Imbra, et al., Nature 323(6088): 555-8, 1986.

Imler et al., Mol Cell Biol 7(7): 2558-67, 1987.

Imperiale et al., Mol Cell Biol 4(5): 875-82, 1984.

Jakobovits et al., Mol Cell Biol 8(6): 2555-61, 1988.

Jameel et al., Mol Cell Biol 6(2): 710-5, 1986.

Jaynes et al., Mol Cell Biol 8(1): 62-70, 1988.

Jiang et al., Mol. Cel. Differ. 1(3): 285-299, 1993.

Jiang et al., Oncogene 11(12): 2477-86, 1995.

Jiang et al., Oncogene 11, 2477, 1995.

Jiang et al., Proc Natl Acad Sci U S A 93(17): 9160-5, 1996.

Jiang et al., Proc. Natl. Acad. Sci. USA. 93, 9160, 1996.

Johnson et al., Mol Cell Biol 9(8): 3393-9, 1989.

Johnson et al., Oncol Rep 5(3): 553-7, 1998.

Jones et al., Cell 13(1): 181-8, 1978.

Kadesch et al., Mol Cell Biol 6(7): 2593-601, 1986.

Kamb et al., Nat Genet 8(1): 23-6, 1994.

Kamb et al., Science 264(5157): 436-40, 1994.

Kandel et al., Cell 66(6): 1095-104, 1991.

Kaneda et al., Science 243(4889): 375-8, 1989.

Kaplitt et al., Nat Genet 8(2): 148-54, 1994.

Karin et al., Mol Cell Biol 7(2): 606-13, 1987.

Karlsson et al., Embo J 5(9): 2377-85, 1986.

Katinka et al., Cell 20(2): 393-9, 1980.

Katinka et al., J Virol 47(1): 244-8, 1983.

Kato et al., J Biol Chem 266(6): 3361-4, 1991.

Kaufman et al., Methods Enzymol 185: 537-66, 1990.

Kawamoto et al., Mol Cell Biol 8(1): 267-72, 1988.

Kawamoto et al., Nucleic Acids Res 17(2): 523-37, 1989.

Kelleher et al., Biotechniques 17(6): 1110-7, 1994.

Kerr et al., Br J Cancer 26(4): 239-57, 1972.

Kiledjian et al., Mol Cell Biol 8(1): 145-52, 1988.

Kim et al., Nature 362(6423): 841-4, 1993.

Klamut et al., Mol Cell Biol 10(1): 193-205, 1990.

Klein et al., Biotechnology 24: 384-6, 1992.

Knappe et al., J. Virol. 74, 3881, 2000.

Koch, et al., Mol Cell Biol 9(1): 303-11, 1989.

Kolmel et al., J Neurooncol 38(2-3): 121-5, 1998.

Kotenko et al., Proc. Natl. Acad. Sci. USA 97, 1695, 2000.

Kotin et al., Proc Natl Acad Sci U S A 87(6): 2211-5, 1990.

Kriegler et al., Blood 63(6): 1348-52, 1984.

Kriegler et al., Cell 38(2): 483-91, 1984.

Kriegler et al., Mol Cell Biol 3(3): 325-39, 1983.

Kuhl et al., Cell 50(7): 1057-69, 1987.

Kunz et al., Nucleic Acids Res 17(3): 1121-38, 1989.

LaFace et al., Virology 162(2): 483-6, 19881

Larsen et al., J Biol Chem 261(31): 14373-6, 1986.

Laspia et al., Cell 59(2): 283-92, 1989.

Latimer et al., Mol Cell Biol 10(2): 760-9, 1990.

Laughlin et al., J Virol 60(2): 515-24. 1986.

Le Gal La Salle et al., Science 259(5097): 988-90, 1993.

Lebkowski et al., Mol Cell Biol 8(10): 3988-96, 1988.

Lee et al., Nature 294(5838): 228-32, 1981.

Lee et al., Nucleic Acids Res 12(10): 4191-206, 1984.

Levine et al., Nature 351(6326): 453-6, 1991.

Levine, Annu Rev Biochem 62: 623-51, 1993.

Levrero et al., Gene 101(2): 195-202, 1991.

Liebermann et al., Int J Oncol 12(3): 685-700, 1998.

Lin et al., Mol Cell Biol 10(2): 850-3, 1990.

Liu et al., J Biol Chem 270(42): 24864-70, 1995.

Luo et al., Exp Hematol 23(12): 1261-7, 1995.

Luria et al., Embo J 6(11): 3307-12, 1987.

Lusky et al., Mol Cell Biol 3(6): 1108-22, 1983.

Lusky et al., Proc Natl Acad Sci U S A 83(11): 3609-13, 1986.

Madireddi et al., In Cancer Gene Therapy: Past Achievements and Future Challenges, Habib Kluwer eds. Academic/Plenum Publishers, N.Y., p. 239, 2000.

Madireddi et al., Oncogene. 19, 1362, 2000.

Magi-Galluzzi et al., Anal Quant Cytol Histol 20(5): 343-50, 1998.

Maione et al., Science 247(4938): 77-9, 1990.

Majors et al., Proc Natl Acad Sci U S A 80(19): 5866-70, 1983.

Mangray et al., Front Biosci 3: D 1148-60, 1998.

Mann et al., Cell 33(1): 153-9, 1983.

Markowitz et al., J Virol 62(4): 1120-4, 1988.

Mathiowitz et al., Nature 386(6623): 410-4, 1997.

May et al., Cytokine 3, 204, 1991.

Mayer et al., Cancer Metastasis Rev 17(2): 211-8, 1998.

McCarty et al., J Virol 65(6): 2936-45, 1991.

McLaughlin et al., J Virol 62(6): 1963-73, 1988.

McNeall et al., Gene 76(1): 81-8, 1989.

Mhashilkar et al., Mol. Medicine. 7, 271, 2001.

Miksicek et al., Cell 46(2): 283-90, 1986.

Millauer et al., Nature 367(6463): 576-9, 1994.

Moore et al., Science 248, 1230, 1990.

Mordacq et al., Genes Dev 3(6): 760-9, 1989.

Moreau et al., Nucleic Acids Res 9(22): 6047-68, 1981.

Mori et al., Cancer Res 54(13): 3396-7, 1994.

Mougin et al., Ann Biol Clin (Paris) 56(1): 21-8, 1998.

Muesing et al., Cell 48(4): 691-701, 1987.

Mumby et al., Cell Regul 2(8): 589-98, 1991.

Muzyczka, Curr Top Microbiol Immunol 158: 97-129, 1992.

Natoli et al., Biochem Pharmacol 56(8): 915-20, 1998.

Neuberger et al., Nucleic Acids Res 16(14B): 6713-24, 1988.

Ng et al., Nucleic Acids Res 17(2): 601-15, 1989.

Nicolas et al., Retroviral vectors. Vectors: A survey of molecular cloning vectors and their uses. Rodriguez and Denhardt, Stoneham: 494-513, 1988.

Nicolau et al., Biochim Biophys Acta 721(2): 185-90, 1982.

Nicolau et al., Methods Enzymol 149: 157-76, 1987.

Nielsen et al., Protein Engineering 10, 1, 1997.

Nobori et al., Nature 368(6473): 753-6, 1994.

Ochi et al., Am J Gastroenterol 93(8): 1366-8, 1998.

Ohara, Gan To Kagaku Ryoho 25(6): 823-8, 1998.

Ohi et al., Gene 89(2): 279-82, 1990.

Okamoto et al., Proc Natl Acad Sci U S A 91(23): 11045-9, 1994.

Ondek et al., Embo J 6(4): 1017-25, 1987.

Ono et al., Pigment Cell Res 10(3): 168-75, 1997.

Orlow et al., Cancer Res 54(11): 2848-51, 1994.

Ornitz et al., Mol Cell Biol 7(10): 3466-72, 1987.

Page et al., Anticancer Res 18(4A): 2313-6, 1998.

Pai et al., Oncol Res 10(6): 295-300, 1998.

Palmiter et al., Cell 29(2): 701-10, 1982.

Parajuli et al., J Med Invest 44(3-4): 205-10, 1998.

Parangi et al., Proc Natl Acad Sci U S A 93(5): 2002-7, 1996.

Paskind et al., Virology 67(1): 242-8, 1975.

Pech et al., Mol Cell Biol 9(2): 396-405, 1989.

Pelletier et al., Nature 334(6180): 320-5, 1988.

Perales et al., Proc Natl Acad Sci U S A 91(9): 4086-90, 1994.

Perez-Stable et al., Mol Cell Biol 10(3): 1116-25, 1990.

Philip et al., J Biol Chem 268(22): 16087-90, 1993.

Picard et al., Embo J 4(11): 2831-8, 1985.

Pietras et al., Oncogene 17(17): 2235-49, 1998.

Pinkert et al., Genes Dev 1(3): 268-76, 1987.

Ponta et al., Proc Natl Acad Sci U S A 82(4): 1020-4, 1985.

Potter et al., Proc Natl Acad Sci U S A 81(22): 7161-5, 1984.

Qin et al., Proc Natl Acad Sci U S A 95(24): 14411-6.

Queen et al., Cell 33(3): 741-8.

Queen et al., Immunol Rev 89: 49-68, 1986.

Quinn et al., Mol Cell Biol 9(11): 4713-21, 1989.

Racher, Biotechnology Techniques 9: 169-174, 1995.

Ragot et al., Nature 361(6413): 647-50, 1993.

Rastinejad et al., Cell 56(3): 345-55, 1989.

Redondo et al., Science 247(4947): 1225-9, 1990.

Reisman et al., Oncogene 4(8): 945-53, 1989.

Renan, Radiother Oncol 19(3): 197-218, 1990.

Resendez et al., Mol Cell Biol 8(10): 4579-84, 1988.

Rich et al., Hum Gene Ther 4(4): 461-76, 1993.

Ridgeway, Mammalian expression vectors. Vectros: A survey of molecular cloning vectors and their uses. Rodriguez and Denhardt, Stoneham: 467-492, 1988.

Rippe et al., Mol Cell Biol 10(2): 689-95, 1990.

Rippe et al., Mol Cell Biol 9(5): 2224-7, 1989.

Rittling et al., Nucleic Acids Res 17(4): 1619-33, 1989.

Rosenfeld et al., Cell 68(1): 143-55, 1992.

Rosenfeld et al., Science 252(5004): 431-4, 1991.

Roux et al., Proc Natl Acad Sci U S A 86(23): 9079-83, 1989.

Saeki et al., Gene Therapy 7, 2051, 2000.

Sakai et al., Proc Natl Acad Sci U S A 85(24): 9456-60, 1988.

Sakai et al., Proc Soc Exp Biol Med 205(3): 236-42, 1994.

Sambrook et al., In: Molecular Cloning: A Laboratory Manual, 2nd Ed., Cold Spring Harbor Press, Cold Spring Harbor, N.Y., 1989.

Samulski et al., Embo J 10(12): 3941-50, 1991.

Samulski et al., J Virol 63(9): 3822-8, 1989.

Santerre et al., Gene 30(1-3): 147-56, 1984.

Schaefer et al., J. Immunol. 166, 5859, 2001.

Schaffner et al., J Mol Biol 201(1): 81-90, 1988.

Searle et al., Mol Cell Biol 5(6): 1480-9, 1985.

Serrano et al., Nature 366(6456): 704-7, 1993.

Serrano et al., Science 267(5195): 249-52, 1995.

Sharp et al., Cell 59(2): 229-30, 1989.

Shaul et al., Embo J 6(7): 1913-20, 1987.

Shelling et al., Gene Ther 1(3): 165-9, 1994.

Sherman et al., Proc Natl Acad Sci U S A 86(17): 6739-43, 1989.

Simpson et al., Gastroenterology 115(4): 849-55, 1998.

Sleigh et al., Embo J 4(13B): 3831-7, 1985.

Soddu et al., Cytokines Cell Mol Ther 4(3): 177-85, 1998.

Solodin et al., Biochemistry 34(41): 13537-44, 1995.

Solyanik et al., Cell Prolif 28(5): 263-78, 1995.

Soo et al., J. Cell. Biochem. 74, 1, 1999.

Spalholz et al., Cell 42(1): 183-91, 1985.

Spandau et al., J Virol 62(2): 427-34, 1988.

Spandidos et al., Embo J 2(7): 1193-9, 1983.

Srivastava et al., Cancer Chemother Pharmacol 42(6): 483-90, 1998.

Steinman et al., Annu Rev Immunol 9: 271-96, 1991.

Stephens et al., Biochem J 248(1): 1-11,1987.

Stokke et al., Cell Prolif 30(5): 197-218, 1997.

Stratford-Perricaudet et al., Gene transfer into animals: the promise of adenovirus. Human Gene Transfer. Cohen-Haguenauer and Boiron. France, John Libbey Eurotext: 51-61, 1991.

Stratford-Perricaudet et al., Hum Gene Ther 1(3): 241-56, 1990.

Strieter et al., Biochem Biophys Res Commun 210(1): 51-7, 1995.

Stuart et al., Nature 317(6040): 828-31, 1985.

Su et al., Proc Natl Acad Sci U S A 95(24): 14400-5, 1998.

Su et al., Proc. Natl. Acad. Sci. USA. 95, 14400, 1998.

Sullivan et al., Mol Cell Biol 7(9): 3315-9, 1987.

Swartzendruber et al., J Cell Physiol 85(2 Pt 1): 179-87, 1975.

Takebe et al., Mol Cell Biol 8(1): 466-72, 1988.

Tavernier et al., Nature 301(5901): 634-6, 1983.

Taylor et al., Mol Cell Biol 10(1): 165-75, 1990.

Taylor et al., Mol Cell Biol 10(1): 176-83, 1990.

Taylor, et al., J Biol Chem 264(27): 16160-4, 1989.

Temin, Retrovirus vectors for gene transfer: Efficient integration into and expression of exogenous DNA in vertebrate cell genome. Gene Transfer. Kucherlapati. N.Y., PlenumPress: 149-188, 1986.

Theobald et al., Proc Natl Acad Sci U S A 92(26): 11993-7, 1995.

Thierry et al., Proc Natl Acad Sci U S A 92(21): 9742-6, 1995.

Thiesen et al., J Virol 62(2): 614-8, 1988.

Top et al., J Infect Dis 124(2): 155-60, 1971.

Tratschin et al., Mol Cell Biol 4(10): 2072-81, 1984.

Tratschin et al., Mol Cell Biol 5(11): 3251-60, 1985.

Treisman et al., Cell 42(3): 889-902, 1985.

Tronche et al., Mol Biol Med 7(2): 173-85, 1990.

Tronche et al., Mol Cell Biol 9(11): 4759-66, 1989.

Trudel et al., Genes Dev 1(9): 954-61, 1987.

Tsujimoto et al., Curr Top Microbiol Immunol 141: 337-40, 1988.

Tsujimoto et al., Science 228(4706): 1440-3, 1985.

Tsukamoto et al., Nat Genet 9(3): 243-8, 1995.

Tur-Kaspa et al., Mol Cell Biol 6(2): 716-8, 1986.

Tyndall et al., Nucleic Acids Res 9(23): 6231-50, 1981.

Van Cott et al., Transgenic Res 6(3): 203-12, 1997.

Vasseur et al., Proc Natl Acad Sci U S A 77(2): 1068-72, 1980.

Voest et al., J Natl Cancer Inst 87(8): 581-6, 1995.

Vogelstein et al., Cell 70(4): 523-6, 1992.

Wagner et al., Science 260(5113): 1510-3, 1993.

Wahlstrom et al., Mol Endocrinol 6(7): 1013-22, 1992.

Walsh et al., J Clin Invest 94(4): 1440-8, 1994.

Wang et al., Cell 47(2): 241-7, 1986.

Watanabe et al., Exp Cell Res 230(1): 76-83, 1997.

Weber et al., Cell 36(4): 983-92, 1984.

Wei et al., Gene Ther 1(4): 261-8, 1994.

Weinberg et al., Science 254(5035): 1138-46, 1991.

Winoto et al., Embo J 8(3): 729-33, 1989.

Wong et al., Gene 10(2): 87-94, 1980.

Wu et al., Biochemistry 27(3): 887-92, 1988.

Wu et al., J Biol Chem 1988 Jan 5;263(1):588].” J Biol Chem 262(10): 4429-32, 1987.

Xie et al., J. Biol. Chem. 275, 31335, 2000.

Yamamoto et al., Cold Spring Harb Symp Quant Biol 47 Pt 2: 977-84, 1983.

Yang et al., J Virol 68(8): 4847-56, 1994.

Yang et al., Proc Natl Acad Sci U S A 87(24): 9568-72, 1990.

Yang, et al., Gene Ther 4(9): 950-60, 1997.

Yano et al., J Med Invest 44(3-4): 185-91, 1998.

Yanuck et al., Cancer Res 53(14): 3257-61, 1993.

Yeom et al., Development 122(3): 881-94, 1996.

Yoder et al., Blood 82(Suppl.): 347A, 1994.

Yoneda et al., J Natl Cancer Inst 90(6): 447-54, 1998.

Yutzey et al., Mol Cell Biol 9(4): 1397-405, 1989.

Zhang et al., J. Biol. Chem. 275, 24436, 2000.

Zhou et al., Exp Hematol 21(7): 928-33, 1993.

Zhou et al., J Exp Med 179(6): 1867-75, 1994.

Zhu et al., Science 2

What is claimed is:

1. A method of inhibiting angiogenesis in a patient in need of such treatment comprising administering to the patient a human MDA-7 polypeptide or a nucleic acid expressing the human MDA-7 polypeptide in eukaryotic cells, whereby the MDA-7 polypeptide inhibits angiogenesis in the patient.

2. The method of claim 1, wherein said patient exhibits an angiogenesis-related disease.

3. The method of claim 2, wherein the angiogenesis-related disease is further defined as angiogenesis-dependent cancer, a benign tumor, rheumatoid arthritis, psoriasis, an ocular angiogenic disease, Osler-Webber Syndrome, myocardial angiogenesis, plaque neovascularization, a telangiectasia, hemophiliac joint, angiofibroma, wound granulation, cat scratch disease, an ulcer, an intestinal adhesion, atherosclerosis, scleroderma, or a hypertrophic scar.

4. The method of claim 3, wherein angiogenesis-dependent cancer is further defined as a solid tumor, leukemia, or a tumor metastasis.

5. The method of claim 3, wherein the benign tumor is further defined as a hemangioma, a neuroma, a neurofibroma, a trachoma, uterine fibroid, hamartoma, teratoma, or a pyogenic granuloma.

6. The method of claim 2 wherein the ocular angiogenic disease is further defined as diabetic retinopathy, retinopathy of prematurity, macular degeneration, corneal graft rejection, neovascular glaucoma, retrolental fibroplasia, or Rubeosis.

7. The method of claim 1, wherein the nucleic acid is an expression vector.

8. The method of claim 7, wherein the expression vector is a viral vector.

9. The method of claim 8, wherein the viral vector is administered at between 10³ and 10¹³ pfu.

10. The method of claim 8, wherein said viral vector is an adenoviral vector, a retroviral vector, a vaccinia viral vector, an adeno-associated viral vector, a polyoma viral vector, or a herpesviral vector.

11. The method of claim 8, wherein said viral vector is an adenoviral vector.

12. The method of claim 1, wherein said nucleic acid further comprises a CMV IE, dectin-1, dectin-2, human CD11 c, F4/80, SM22 or MHC class II promoter.

13. The method of claim 1, wherein the MDA-7 polypeptide or nucleic acid is administered to the patient by direct injection into an area in need of inhibition of angiogenesis.

14. The method of claim 13, wherein the patient is administered multiple injections.

15. The method of claim 13, wherein the injection is performed locally to a disease site.

16. The method of claim 13, wherein the injection is performed regionally to a disease site.

17. The method of claim 13, wherein the injection is performed distally to a disease site.

18. The method of claim 1, wherein the MDA polypeptide or the nucleic acid is administered to the patient by continuous infusion.

19. The method of claim 1, wherein the MDA polypeptide or the nucleic acid is administered to the patient by intravenous injection.

20. The method of claim 1, wherein the MDA polypeptide or the nucleic acid is administered prior to or after surgery.

21. The method of claim 1, wherein the MDA polypeptide or the nucleic acid is administered before chemotherapy, immunotherapy, or radiotherapy.

22. The method of claim 1, wherein the MDA polypeptide or the nucleic acid is administered during chemotherapy, immunotherapy, or radiotherapy.

23. The method of claim 1, wherein the MDA polypeptide or the nucleic acid is administered after chemotherapy, immunotherapy, or radiotherapy.

24. The method of claim 1, wherein the patient is a human.

25. The method of claim 1, wherein the MDA polypeptide comprises amino acids from 1 to 206 of SEQ ID NO:2.

26. The method of claim 1, wherein the MDA polypeptide comprises amino acids from 49 to 206 of SEQ ID NO:2.

27. The method of claim 1, wherein the MDA polypeptide comprises amino acids from 75 to 206 of SEQ ID NO:1.

28. The method of claim 1, wherein the MDA polypeptide comprises amino acids from about 100 to about 206 of SEQ ID NO:2.

29. The method of claim 1, wherein the MDA polypeptide comprises amino acids from 125 to 206 of SEQ ID NO:2.

30. The method of claim 1, wherein the MDA polypeptide comprises amino acids from 150 to 206 of SEQ ID NO:2.

31. The method of claim 1, wherein the MDA polypeptide comprises amino acids from 175 to 206 of SEQ ID NO:2.

32. The method of claim 1, wherein the MDA polypeptide comprises amino acids from 182 to 206 of SEQ ID NO:2.

33. The method of claim 1, wherein the MDA polypeptide comprises a secretory signal.

34. The method of claim 33, wherein the secretory signal is further defined as a positively charged N-terminal region in combination with a hydrophobic core.

35. The method of claim 1, wherein the patient is a cancer patient.

36. A method of inhibiting endothelial cell differentiation in a patient comprising administering to the patient an effective amount of a human MDA-7 polypeptide or a nucleic acid molecule expressing the human MDA-7 polypeptide.

37. The method of claim 36, wherein a chemotherapeutic agent is administered prior to administration of the MDA-7 polypeptide or the nucleic acid molecule.

38. The method of claim 36 wherein a chemotherapeutic agent is administered after administration of the MDA-7 polypeptide or the nucleic acid molecule.

39. The method of claim 36, wherein the chemotherapeutic agent is a DNA damaging agent.

40. The method of claim 39, wherein the DNA damaging agent is gamma-irradiation, X-rays, UV-irradiation, microwaves, electronic emissions, adriamycin, 5-fluorouracil (5FU), etoposide (VP-16), camptothecin, actinomycin-D, mitomycin C, cisplatin (CDDP), or hydrogen peroxide.

41. The method of claim 38, wherein the chemotherapeutic agent is a cisplatin (CDDP), carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxol, transplatinum, 5-fluorouracil, vincristin, vinblastin, methotrexate, or analog or derivative variant thereof.

42. The method of claim 36, wherein the nucleic acid is comprised within a viral vector.

43. The method of claim 36, wherein the nucleic acid is comprised in a lipid composition.

44. A method for promoting an immune response in a patient comprising providing to the subject an amount of an MDA-7 polypeptide effective to induce an immune response in the patient.

45. The method of claim 44, further comprising administering to the patient an antigen against which an immune response is promoted.

46. The method of claim 45, wherein the antigen is a tumor antigen, microbial antigen, viral antigen, or fungal antigen.

47. The method of claim 46, wherein the antigen is a tumor antigen.

48. The method of claim 46, wherein the antigen is a microbial antigen.

49. The method of claim 46, wherein the antigen is a viral antigen.

50. The method of claim 46, wherein the antigen is a fungal antigen.

51. The method of claim 47, wherein the tumor antigen is PSA, CEA, MART, MAGE1, MAGE 3, gp100, BAGE, GAGE, TRP-1, TRP-2, or PMSA.

52. The method of claim 44, wherein the MDA-7 is provided to the patient by administering to the subject an expression construct comprising a nucleic acid sequence encoding at least 50 contiguous amino acids of SEQ ID NO:2, wherein the nucleic acid sequence is under the transcriptional control of a promoter.

53. The method of claim 52, wherein the expression construct is a viral vector.

54. The method of claim 53, wherein the viral vector is an adenovirus vector, an adeno-associated virus vector, a herpesvirus vector, a retrovirus vector, a lentivirus vector, a vaccinia virus vector, or a polyoma vector.

55. The method of claim 44, wherein the antigen is provided to the patient by administering to the patient an expression construct comprising a nucleic acid sequence encoding the antigen, wherein the nucleic acid sequence is under the transcriptional control of a promoter.

56. The method of claim 44, wherein the MDA-7, antigen, or both are provided to the patient more than one time.

57. The method of claim 44, wherein the MDA-7, antigen, or both are provided to the patient intravenously, directly, intraperitoneally, regionally, systemically, or orally.

58. The method of claim 44, wherein the MDA-7 and antigen are provided to the subject at the same time.

59. A method of inducing expression of IL-6, IFNγ, or TNFα in a cell comprising administering to the cell an effective amount of an MDA-7 polypeptide or a nucleic acid expressing the MDA-7 polypeptide.

60. The method of claim 59, wherein expression of IL-6 is induced.

61. The method of claim 59, wherein expression of TNFα is induced.

62. The method of claim 59, wherein expression of IFNγ is induced.

63. The method of claim 59, wherein the cell is in a patient.

64. A method of reducing cell damage from chemotherapy or radiotherapy in a cancer patient comprising administering to the patient an effective amount of a human MDA-7 polypeptide or a nucleic acid expressing the human MDA-7 polypeptide.

65. The method of claim 64, wherein the MDA polypeptide or nucleic acid is administered to the patient when chemotherapy or radiotherapys is administered.

66. The method of claim 64, wherein the MDA polypeptide or nucleic acid is administered to the patient after chemotherapy or radiotherapy is administered.

67. The method of claim 54, wherein the MDA polypeptide or nucleic acid is administered to the patient more than one time.
